(19) United States
(12) Patent Application Publication
(10) Pub. No.: US 2025/0084054 A1
(43) Pub. Date: Mar. 13, 2025

(54) SMALL MOLECULE INHIBITORS OF MAMMALIAN SLC6A19 FUNCTION

(71) Applicant: Janna Therapeutics Inc, Boston, MA (US)

(72) Inventors: Dean G. Brown, Hollis, NH (US); Giovanni Muncipinto, Cambridge, MA (US); Joshua E. Zweig, Boston, MA (US); Ryan A. Hollibaugh, Quincy, MA (US); Nicholas Pullen, Arlington, MA (US); Mitchell T. Antalek, Medford, MA (US); Long V. Nguyen, Boston, MA (US); John A. Malona, Lexington, MA (US)

(21) Appl. No.: 18/723,012

(22) PCT Filed: Dec. 22, 2022
(86) PCT No.: PCT/US2022/053798

§ 371 Date: Jun. 21, 2024

Related U.S. Application Data
(60) Provisional application No. 63/292,818, filed on Dec. 22, 2021.

(51) Int. Cl. C07D 401/04; A61K 31/506; A61K 31/53; C07D 413/14

(52) U.S. Cl. CPC C07D 401/04; A61K 31/506; A61K 31/53; C07D 413/14

(57) ABSTRACT

Disclosed are compounds, compositions, and methods useful for treating or preventing a disease or disorder associated with abnormal levels of amino acids by modulation of SLC6A19 transport.

### RELATED APPLICATION

This application claims the benefit of priority to U.S. Provisional Patent Application No. 63/292,818, filed Dec. 22, 2021.

### BACKGROUND

Phenylketonuria (PKU) is an inborn error of metabolism caused by mutations in phenylalanine hydroxylase (PAH), the enzyme responsible for metabolizing phenylalanine. PKU is an autosomal recessive metabolic disorder in which phenylalanine is not properly metabolized and results in abnormally high levels of plasma phenylalanine. People who have PKU have abnormally high blood levels of phenylalanine, which if untreated can lead to irreversible neurological damage resulting in a spectrum of complications such as intellectual disabilities, seizures, neurodevelopmental and behavioral disorders. PKU is difficult to treat because blood levels of phenylalanine are directly related to diet. Patients must adhere to a life-long and strict diet that impacts all aspects of patients' lives. Current standard of care are enzyme co-factor and enzyme substitution therapy but these therapies are not effective in all patients, and carry potential risk for adverse events.

The enzyme responsible for metabolizing phenylalanine, and thus maintaining phenylalanine homeostasis is phenylalanine hydroxylase (PAH). Loss-of-function (LOF) mutations at PAH gene at chromosome 12q23.2 are known to cause most forms of PKU. These LOF mutations resulting in PKU can be diagnosed as classical PKU (the most severe form), and “mild PKU” or “hyperphe” a less severe form. In addition to PAH, mutations in other enzymes that affect phenylalanine metabolism, such as dihydropteridine reductase (DHPR), the enzyme responsible for synthesis of co-factors required for PAH activity, may also result in elevated levels of phenylalanine. In addition to diet, blood amino acid levels, including phenylalanine, are regulated by SLC6A19. SCL6A19 is located in the proximal tubule of the kidney and is responsible for reabsorption of amino acids back into the blood.

### SUMMARY

One aspect of the invention provides compounds, compositions, and methods useful for treating or preventing a disease or disorder associated with abnormal levels of amino acids by modulation of SLC6A19 transport.

Accordingly, provided herein is a compound having the structure of Formula (I):

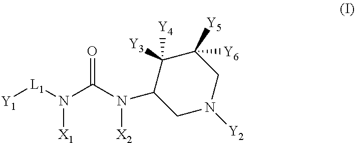

wherein:
L₁ is absent or selected from -alkyl-, -cycloalkyl-, and -heteroaryl-CH₂—;
X₁ and X₂ are independently selected from —H, alkyl, cycloalkyl, and alkyl-cycloalkyl; provided that X₁ and X₂ are not both —H;
Y₁ is optionally substituted aryl; and
Y₂ is selected from optionally substituted pyridonyl, optionally substituted pyrimidinoyl, optionally substituted pyrazinonyl, optionally substituted triazinonyl, and optionally substituted quinazolinonyl;
Y₃, Y₄, Y₅, and Y₆ are independently selected from —H and halide;
or a pharmaceutically acceptable salt thereof.

Another aspect of the invention relates to methods of treating or preventing a disease or disorder associated with a genetic defect in phenylalanine hydroxylase in a subject in need thereof comprising administering to the subject an effective amount of a compound of Formula (I).

Another aspect of the invention relates to methods of treating or preventing phenylketonuria, hyperphenylalaninemia, tyrosinemia, nonketotic hyperglycinemia, isovaleric academia, methylmalonic academia, propionic academia, maple syrup urine disease, DNAJC12 deficiency, urea cycle disorders, or hyperammonemia in a subject in need thereof comprising administering to the subject an effective amount of a compound of Formula (I).

Another aspect of the invention relates to methods of modulating SLC6A19 transport in a subject in need thereof comprising administering to the subject an effective amount of a compound of Formula (I).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the detailed description, and from the claims.

### BRIEF DESCRIPTION OF THE DRAWINGS

FIG. 1 is a table summarizing isoleucine transport data for exemplary compounds of the invention. A=IC₅₀<500 nM; B=IC₅₀ 500 nM-1,500 nM; C=IC₅₀>1,500 nM-5,000 nM; and D=IC₅₀>5,000 nM-10,000 nM.

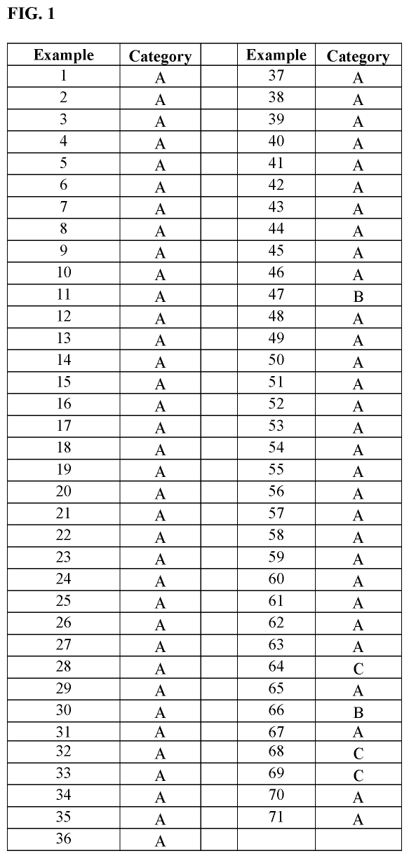

### DETAILED DESCRIPTION

### Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

In order for the present invention to be more readily understood, certain terms and phrases are defined below and throughout the specification.

The articles “a” and “an” are used herein to refer to one or to more than one (i.e., to at least one) of the grammatical object of the article. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Certain compounds contained in compositions of the present invention may exist in particular geometric or stereoisomeric forms. In addition, polymers of the present invention may also be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in “atropisomeric” forms or as “atropisomers.” Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from a mixture of isomers. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

If, for instance, a particular enantiomer of compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

The term “tautomer” as used herein means structural isomers that exist in equilibrium resulting from the migration of a hydrogen atom. For example, the two tautomers of 2-pyrimidinone are recited below. A single tautomer may be provided in a structural representation of a given compound. However, the present invention contemplates all such tautomers of a given compound.

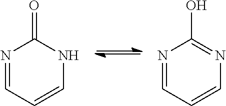 [SMILES: O=c1nccc[nH]1 ; Oc1ncccn1]

Percent purity by mole fraction is the ratio of the moles of the enantiomer (or diastereomer) or over the moles of the enantiomer (or diastereomer) plus the moles of its optical isomer. When the stereochemistry of a disclosed compound is named or depicted by structure, the named or depicted stereoisomer is at least about 60%, about 70%, about 80%, about 90%, about 99% or about 99.9% by mole fraction pure relative to the other stereoisomers. When a single enantiomer is named or depicted by structure, the depicted or named enantiomer is at least about 60%, about 70%, about 80%, about 90%, about 99% or about 99.9% by mole fraction pure. When a single diastereomer is named or depicted by structure, the depicted or named diastereomer is at least about 60%, about 70%, about 80%, about 90%, about 99% or about 99.9% by mole fraction pure.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s) or mixtures of diastereomers in which one or more diastereomer is enriched relative to the other diastereomers. The invention embraces all of these forms.

Structures depicted herein are also meant to include compounds that differ only in the presence of one or more isotopically enriched atoms. For example, compounds produced by the replacement of a hydrogen with deuterium or tritium, or of a carbon with a ¹³C- or ¹⁴C-enriched carbon are within the scope of this invention.

The term “prodrug” as used herein encompasses compounds that, under physiological conditions, are converted into therapeutically active agents. A common method for making a prodrug is to include selected moieties that are hydrolyzed under physiological conditions to reveal the desired molecule. In other embodiments, the prodrug is converted by an enzymatic activity of the host animal.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation, not injurious to the patient, and substantially non-pyrogenic. Some examples of materials which can serve as pharmaceutically acceptable carriers include: (1) sugars, such as lactose, glucose, and sucrose; (2) starches, such as corn starch and potato starch; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. In certain embodiments, pharmaceutical compositions of the present invention are non-pyrogenic, i.e., do not induce significant temperature elevations when administered to a patient.

The term “pharmaceutically acceptable salts” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate salts, and the like. (See, for example, Berge et al. (1977) “Pharmaceutical Salts”, J. Pharm. Sci. 66:1-19.)

In other cases, the compounds useful in the methods of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

The term “pharmaceutically acceptable cocrystals” refers to solid coformers that do not form formal ionic interactions with the small molecule.

A “therapeutically effective amount” (or “effective amount”) of a compound with respect to use in treatment, refers to an amount of the compound in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient” or “subject” refers to a mammal in need of a particular treatment. In certain embodiments, a patient is a primate, canine, feline, or equine. In certain embodiments, a patient is a human.

An aliphatic chain comprises the classes of alkyl, alkenyl and alkynyl defined below. A straight aliphatic chain is limited to unbranched carbon chain moieties. As used herein, the term “aliphatic group” refers to a straight chain, branched-chain, or cyclic aliphatic hydrocarbon group and includes saturated and unsaturated aliphatic groups, such as an alkyl group, an alkenyl group, or an alkynyl group.

“Alkyl” refers to a fully saturated cyclic or acyclic, branched or unbranched carbon chain moiety having the number of carbon atoms specified, or up to 30 carbon atoms if no specification is made. For example, alkyl of 1 to 8 carbon atoms refers to moieties such as methyl, ethyl, propyl, butyl, pentyl, hexyl, heptyl, and octyl, and those moieties which are positional isomers of these moieties. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain embodiments, a straight chain or branched chain alkyl has 30 or fewer carbon atoms in its backbone (e.g., C₁-C₃₀ for straight chains, C₃-C₃₀ for branched chains), and more preferably 20 or fewer. Alkyl groups may be substituted or unsubstituted.

As used herein, the term “heteroalkyl” refers to an alkyl moiety as hereinbefore defined which contain one or more oxygen, sulfur, nitrogen, phosphorus, or silicon atoms in place of carbon atoms.

As used herein, the term “haloalkyl” refers to an alkyl group as hereinbefore defined substituted with at least one halogen.

As used herein, the term “hydroxyalkyl” refers to an alkyl group as hereinbefore defined substituted with at least one hydroxyl.

As used herein, the term “alkylene” refers to an alkyl group having the specified number of carbons, for example from 2 to 12 carbon atoms, that contains two points of attachment to the rest of the compound on its longest carbon chain. Non-limiting examples of alkylene groups include methylene —(CH₂)—, ethylene —(CH₂CH₂)—, n-propylene —(CH₂CH₂CH₂)—, isopropylene —(CH₂CH(CH₃))—, and the like. Alkylene groups can be cyclic or acyclic, branched or unbranched carbon chain moiety, and may be optionally substituted with one or more substituents.

“Cycloalkyl” means mono- or bicyclic or bridged or spirocyclic, or polycyclic saturated carbocyclic rings, each having from 3 to 12 carbon atoms. Preferred cycloalkyls have from 3-10 carbon atoms in their ring structure, and more preferably have 3-6 carbons in the ring structure. Cycloalkyl groups may be substituted or unsubstituted.

As used herein, the term “halocycloalkyl” refers to an cycloalkyl group as hereinbefore defined substituted with at least one halogen.

“Cycloheteroalkyl” refers to an cycloalkyl moiety as hereinbefore defined which contain one or more oxygen, sulfur, nitrogen, phosphorus, or silicon atoms in place of carbon atoms. Preferred cycloheteroalkyls have from 4-8 carbon atoms and heteroatoms in their ring structure, and more preferably have 4-6 carbons and heteroatoms in the ring structure. Cycloheteroalkyl groups may be substituted or unsubstituted.

Unless the number of carbons is otherwise specified, “lower alkyl,” as used herein, means an alkyl group, as defined above, but having from one to ten carbons, more preferably from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, preferred alkyl groups are lower alkyls. In certain embodiments, a substituent designated herein as alkyl is a lower alkyl. “Alkenyl” refers to any cyclic or acyclic, branched or unbranched unsaturated carbon chain moiety having the number of carbon atoms specified, or up to 26 carbon atoms if no limitation on the number of carbon atoms is specified; and having one or more double bonds in the moiety. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosoenyl, docosenyl, tricosenyl, and tetracosenyl, in their various isomeric forms, where the unsaturated bond(s) can be located anywhere in the moiety and can have either the (Z) or the (E) configuration about the double bond(s).

“Alkynyl” refers to hydrocarbyl moieties of the scope of alkenyl, but having one or more triple bonds in the moiety.

The term “aryl” as used herein includes 3- to 12-membered substituted or unsubstituted single-ring aromatic groups in which each atom of the ring is carbon (i.e., carbocyclic aryl) or where one or more atoms are heteroatoms (i.e., heteroaryl). Preferably, aryl groups include 5- to 12-membered rings, more preferably 6- to 10-membered rings The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Carboycyclic aryl groups include benzene, naphthalene, phenanthrene, phenol, aniline, and the like. Heteroaryl groups include substituted or unsubstituted aromatic 3- to 12-membered ring structures, more preferably 5- to 12-membered rings, more preferably 5- to 10-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like. Aryl and heteroaryl can be monocyclic, bicyclic, or polycyclic.

The term “halo”, “halide”, or “halogen” as used herein means halogen and includes, for example, and without being limited thereto, fluoro, chloro, bromo, iodo and the like, in both radioactive and non-radioactive forms. In a preferred embodiment, halo is selected from the group consisting of fluoro, chloro and bromo.

The terms “heterocyclyl” or “heterocyclic group” refer to 3- to 12-membered ring structures, more preferably 5- to 12-membered rings, more preferably 5- to 10-membered rings, whose ring structures include one to four heteroatoms. Heterocycles can be monocyclic, bicyclic, spirocyclic, or polycyclic. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, and the like.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

As used herein, the definition of each expression, e.g., alkyl, m, n, etc., when it occurs more than once in any structure, is intended to be independent of its definition elsewhere in the same structure.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, a “small molecule” refers to an organic, inorganic, or organometallic compound typically having a molecular weight of less than about 1000. In some embodiments, a small molecule is an organic compound, with a size on the order of 1 nm. In some embodiments, small molecule drugs of the invention encompass oligopeptides and other biomolecules having a molecular weight of less than about 1000.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modulate” includes up-regulation and down-regulation, e.g., enhancing or inhibiting a response.

A “radiopharmaceutical agent,” as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

For purposes of this invention, the chemical elements are identified in accordance with the Periodic Table of the Elements, CAS version, Handbook of Chemistry and Physics, 67th Ed., 1986-87, inside cover.

### Compounds of the Invention

One aspect of the invention relates to a compound of Formula (I):

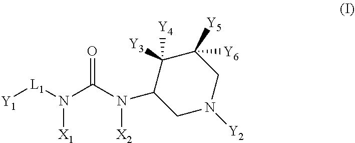

wherein:
L₁ is absent or selected from -alkyl-, -cycloalkyl-, and -heteroaryl-CH₂—;
X₁ and X₂ are independently selected from —H, alkyl, cycloalkyl, and alkyl-cycloalkyl; provided that X₁ and X₂ are not both —H;
Y₁ is optionally substituted aryl; and
Y₂ is selected from optionally substituted pyridonyl, optionally substituted pyrimidinoyl, optionally substituted pyrazinonyl, optionally substituted triazinonyl, and optionally substituted quinazolinonyl;
Y₃, Y₄, Y₅, and Y₆ are independently selected from —H and halide;
or a pharmaceutically acceptable salt thereof.

In certain embodiments, the compound having the structure:

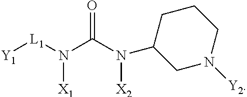

In certain embodiments, one of X₁ and X₂ is —H; and the other of X₁ and X₂ is selected from C₁-C₄ alkyl, cycloalkyl, and alkyl-cycloalkyl.

In certain embodiments, one of X₁ and X₂ is —H; and the other of X₁ and X₂ is selected from C₁-C₄ alkyl and cycloalkyl.

In certain embodiments, X₁ is —H; and X₂ is —CH₃. In other embodiments, X₂ is —H; and X₁ is —CH₃. In other embodiments, X₁ is —H; and X₂ is

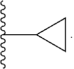 [SMILES: *C1CC1]

In other embodiments, X₂ is —H; and X₁ is

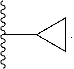 [SMILES: *C1CC1]

In certain embodiments, L₁ is absent.

In certain embodiments, L₁ is selected from —C₁-C₄ alkyl-, -cycloalkyl-, and -heteroaryl-CH₂—.

In certain embodiments, L₁ is —CH₂—. In other embodiments, L₁ is

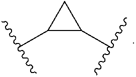 [SMILES: *C1CC1*]

In other embodiments, L₁ is selected from

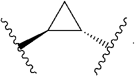 [SMILES: *[C@H]1C[C@@H]1*]

and

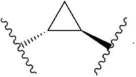 [SMILES: *[C@@H]1C[C@H]1*]

In other embodiments, L₁ is selected from

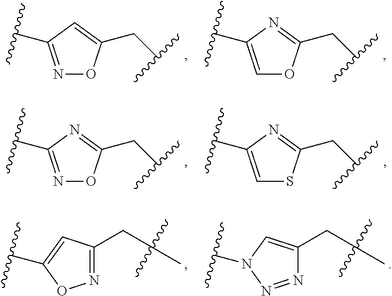 [SMILES: *Cc1cc(*)no1 ; *Cc1nc(*)co1 ; *Cc1nc(*)no1 ; *Cc1nc(*)cs1 ; *Cc1cc(*)on1 ; *Cc1cn(*)nn1]

and

In certain embodiments, the compound having a structure selected from

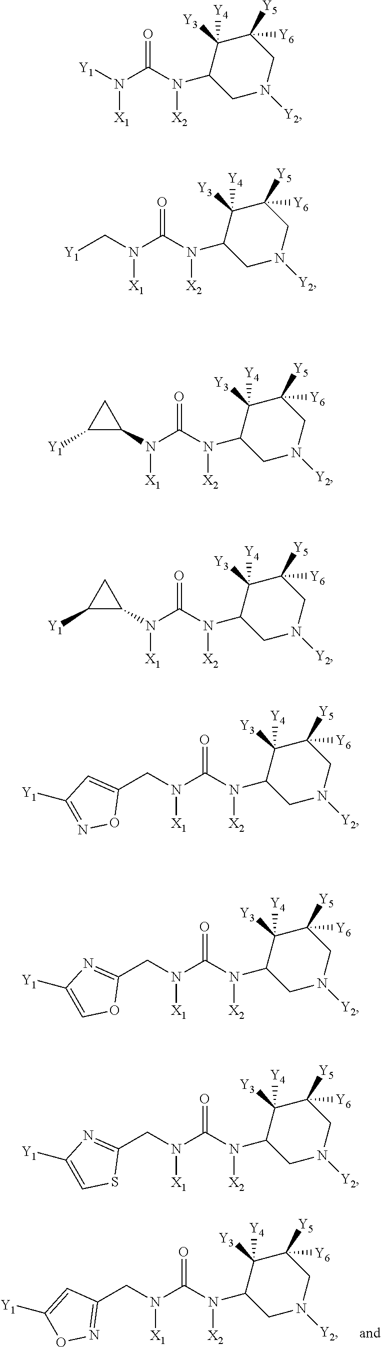

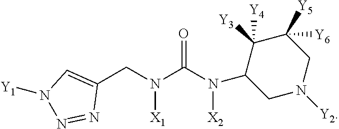

In certain embodiments, the compound having a structure selected from:

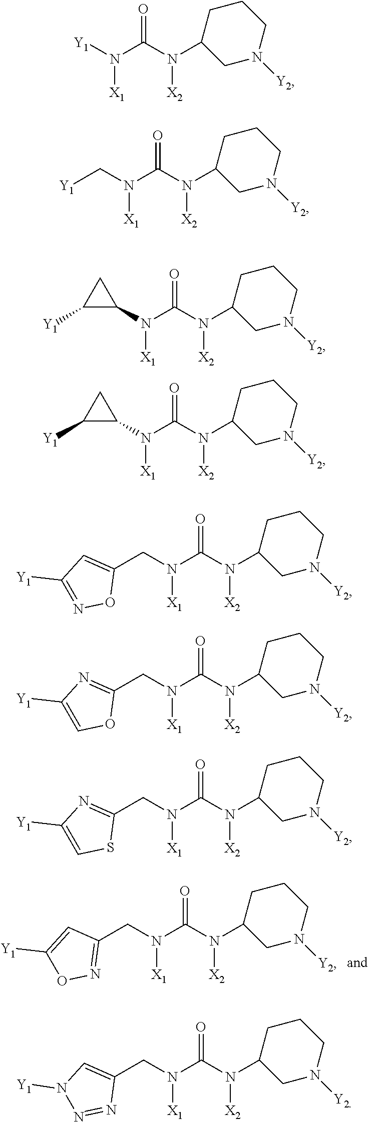

In certain embodiments, Y₁ is unsubstituted aryl.

In certain embodiments, Y₁ is unsubstituted phenyl.

In certain embodiments, wherein Y₁ is substituted aryl.

In certain embodiments, Y₁ is

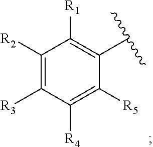

and
R₁, R₂, R₃, R₄, and R₅ are independently selected from —H, halogen, —CN, —CF₃, —CHF₂, —CF₂CH₃, —OCF₃, —OCHF₂, alkyl, haloalkyl, hydroxyalkyl, alkenyl, alkynyl, alkoxy, alkylamino and cycloalkyl; provided that at least one of R₁, R₂, R₃, R₄, and R₅ is not —H.

In certain embodiments, R₁, R₂, R₃, R₄, and R₅ are independently selected from —H, —F, —Cl, —Br, —CN, —CH₃, —CH₂CH₃, —CF₃, —CHF₂, —CF₂CH₃, —C(H)(OH)(CH₃), —OCH₃, —OCF₃, —OCHF₂, and

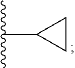 [SMILES: *C1CC1]

provided that at least one of R₁, R₂, R₃, R₄, and R₅ is not —H.

In certain embodiments, R₁, R₂, R₃, R₄, and R₅ are independently selected from —H, —F, —Cl, —Br, —CN, —CH₃, —CH₂CH₃, and

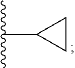 [SMILES: *C1CC1]

provided that at least one of R₁, R₂, R₃, R₄, and R₅ is not —H.

In certain embodiments, two of R₁, R₂, R₃, R₄, and R₅ are not —H.

In certain embodiments, three of R₁, R₂, R₃, R₄, and R₅ are not —H.

In certain embodiments, Y₁ is

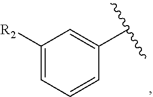

wherein R₂ is selected from —Cl, —Br, —F, —CN, —CH₃, —CH₂CH₃, —OCH₃, and —CF₂CH₃.

In certain embodiments, Y₁ is

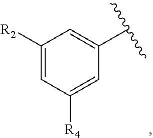

wherein R₂ and R₄ are each independently selected from —Cl, —Br, —F, —CN, —CH₃, —CH₂CH₃, —OCH₃, and —CF₂CH₃.

In certain embodiments, wherein Y₁ is

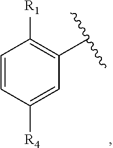

wherein R₁ and R₄ are each independently selected from —Cl, —Br, —F, —CN, —CH₃, —CH₂CH₃, —OCH₃, and —CF₂CH₃. In other embodiments, Y₁ is

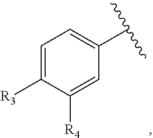

wherein R₃ and R₄ are each independently selected from —Cl, —Br, —F, —CN, —CH₃, —CH₂CH₃, —OCH₃, and —CF₂CH₃.

In certain embodiments, Y₁ is

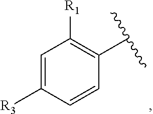

wherein R₁ and R₃ are each independently selected from —Cl, —Br, —F, —CN, —CH₃, —CH₂CH₃, —OCH₃, —OCF₃, —CF₂CH₃, and

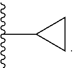 [SMILES: *C1CC1]

In certain embodiments, R₁ is —F; and R₃ is selected from —Cl, —Br, —F, —CN, —CH₃, —CH₂CH₃, —OCH₃, —OCF₃, —CF₂CH₃, and

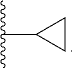 [SMILES: *C1CC1]

In certain embodiments, wherein Y₁ is

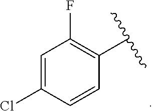 [SMILES: *c1ccc(Cl)cc1F]

In certain embodiments, Y₁ is

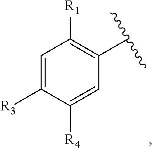

wherein R₁, R₃, and R₄ are each independently selected from —Cl, —Br, —F, —CN, —CH₃, —CH₂CH₃, —OCH₃, —OCF₃, —CF₂CH₃, —C(H)(OH)(CH₃), and

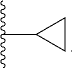 [SMILES: *C1CC1]

In certain embodiments, R₁ is —F; and R₃ and R₄ are each independently selected from —Cl, —Br, —F, —CN, —CH₃, —CH₂CH₃, —OCH₃, —OCF₃, —CF₂CH₃, and

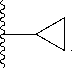 [SMILES: *C1CC1]

In certain embodiments, R₁ is —F; R₃ is —C₁ or —F; and R₄ is selected from —Cl, —Br, —F, —CN, —CH₃, —CH₂CH₃, —OCH₃, —OCF₃, —CF₂CH₃, and

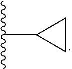 [SMILES: *C1CC1]

In certain embodiments, the compound having the structure selected from:

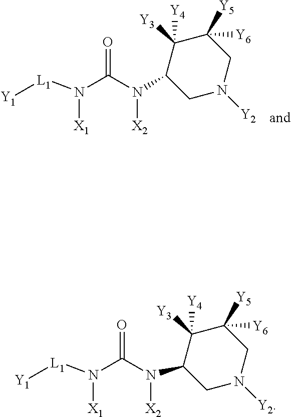

In certain embodiments, the compound having the structure selected from:

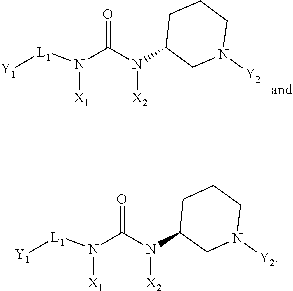

In certain embodiments, Y₂ is selected from unsubstituted pyridonyl, unsubstituted pyrimidinoyl, unsubstituted pyrazinonyl, unsubstituted triazinonyl, and unsubstituted quinazolinonyl,

In certain embodiments, Y₂ is selected from

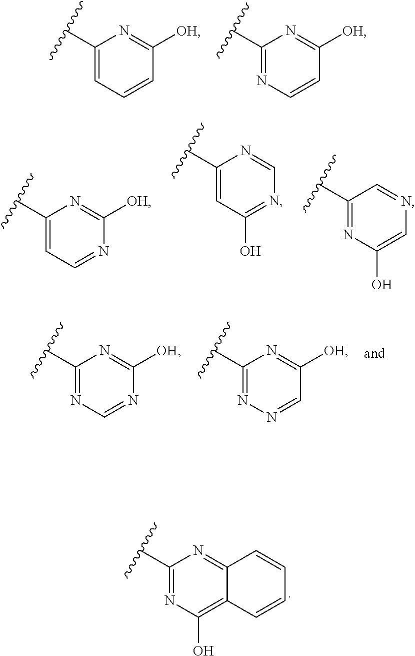

In certain embodiments, Y₂ is selected from substituted pyridonyl, substituted pyrimidinoyl, substituted pyrazinonyl, substituted triazinonyl, and substituted quinazolinonyl,

In certain embodiments, Y₂ is

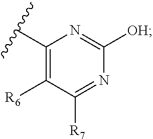

and
R₆ and R₇ are independently selected from —H, halogen, —CN, —OH, —OCF₃, —OCHF₂, —NH₂, alkyl, alkoxy, alkylamino, and cycloalkyl; provided that at least one of R₆ and R₇ is not —H; or R₆ and R₇ taken together with the carbons to which they are bonded form an unsubstituted or substituted fused C₅-C₇ cycloalkyl.

In certain embodiments, Y₂ is

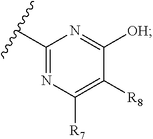

and
R₇ and R₈ are independently selected from —H, halogen, —CN, —OH, —OCF₃, —OCHF₂, —NH₂, alkyl, alkoxy, alkylamino, and cycloalkyl; provided that at least one of R₇ and R₈ is not —H; or R₇ and R₈ taken together with the carbons to which they are bonded form an unsubstituted or substituted fused C₅-C₇ cycloalkyl.

In certain embodiments, Y₂ is

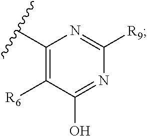

and
R₆ and R₉ are independently selected from —H, halogen, —CN, —OH, —OCF₃, —OCHF₂, —NH₂, alkyl, alkoxy, alkylamino, and cycloalkyl; provided that at least one of R₆ and R₉ is not —H.

In certain embodiments, Y₂ is

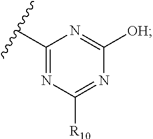

and
R₁₀ is selected from halogen, —CN, —OH, —OCF₃, —OCHF₂, —NH₂, alkyl, alkoxy, alkylamino, and cycloalkyl.

In certain embodiments, Y₂ is

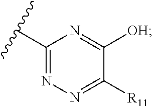

and
R₁ is selected from halogen, —CN, —OH, —OCF₃, —OCHF₂, —NH₂, alkyl, alkoxy, alkylamino, and cycloalkyl.

In certain embodiments, Y₂ is selected from

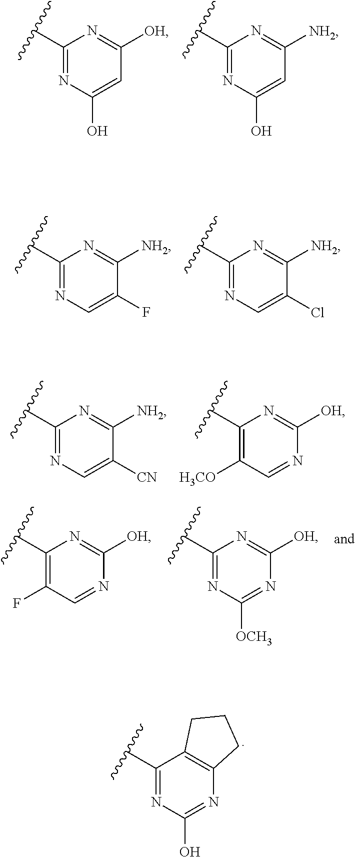

In certain embodiments, Y₂ is an N-substituted pyridonyl, N-substituted pyrimidinoyl, N-substituted pyrazinonyl, N-substituted triazinonyl, or N-substituted quinazolinonyl,

In certain embodiments, Y₂ is an N-alkyl substituted pyridonyl, N-alkyl substituted pyrimidinoyl, N-alkyl substituted pyrazinonyl, N-alkyl substituted triazinonyl, or N-alkyl substituted quinazolinonyl,

In certain embodiments, Y₂ is selected from

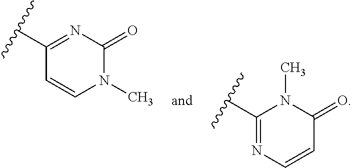

In certain embodiments, the compound having the structure:

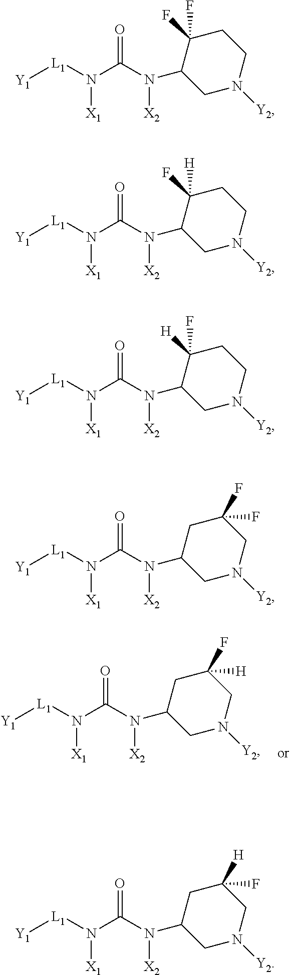

In certain embodiments, Y₃ and Y₄ are both —H or both —F. In other embodiments, Y₃ is —H and Y₄ is —F. In other embodiments, Y₄ is —H and Y₃ is —F.

In certain embodiments, Y₅ and Y₆ are both —H or both —F. In other embodiments, Y₅ is —H and Y₆ is —F. In other embodiments, Y₆ is —H and Y₅ is —F.

In certain embodiments, Y₃, Y₄, Y₅, and Y₆ are each —H.

In certain embodiments, the compound is selected from the following Table 1:

TABLE 1

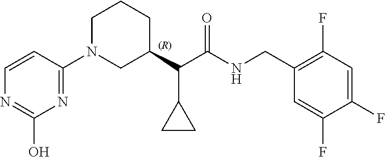

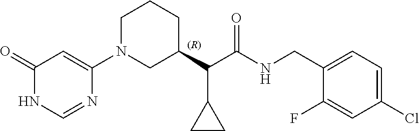

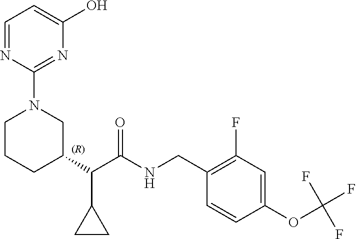

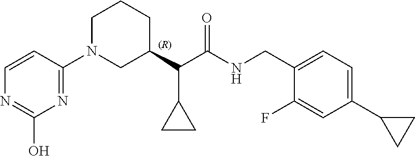

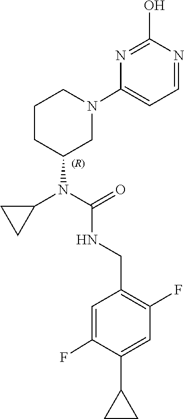

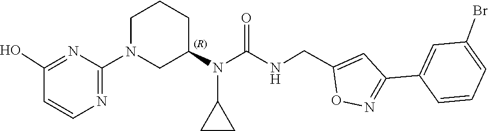

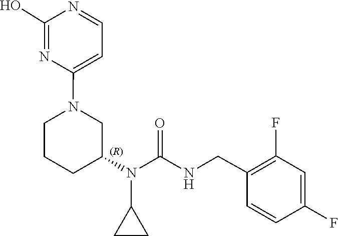

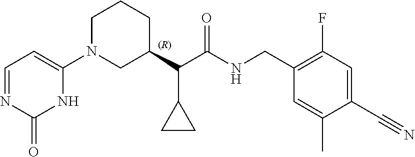

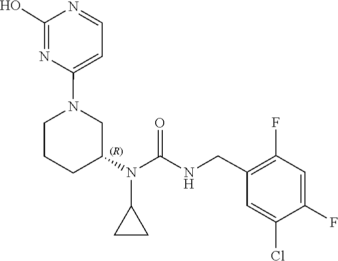

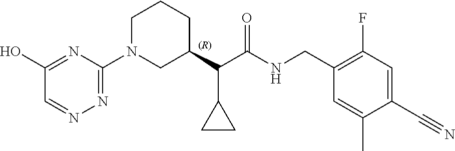

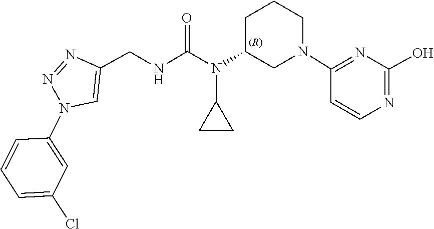

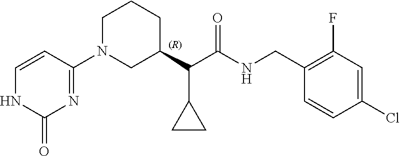

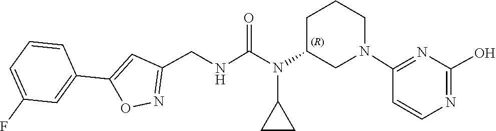

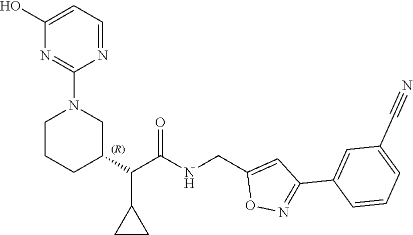

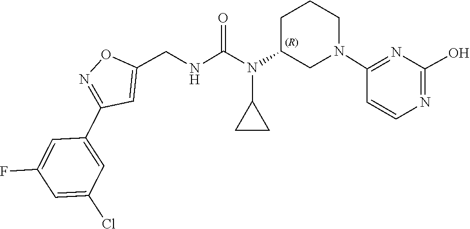

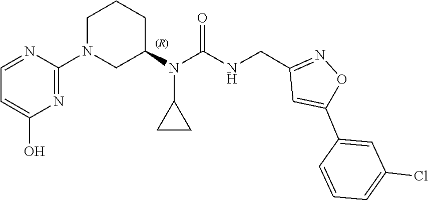

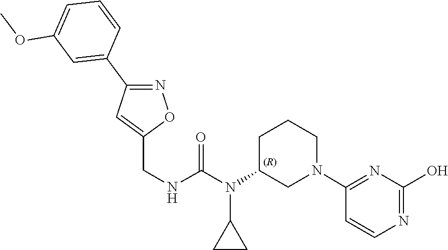

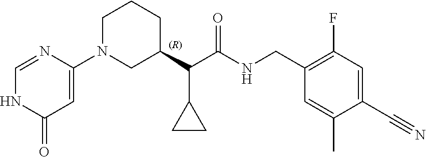

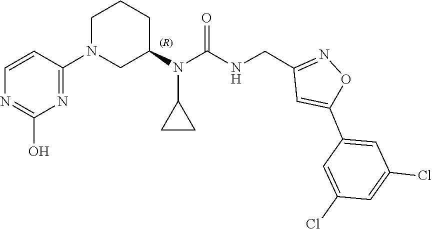

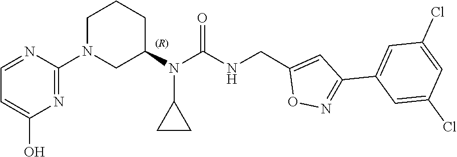

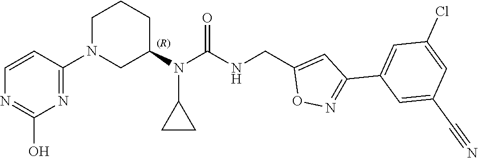

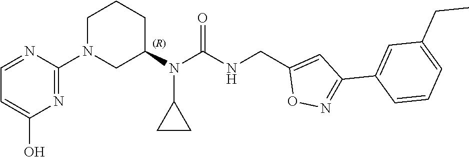

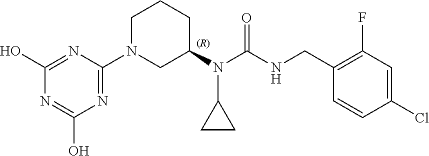

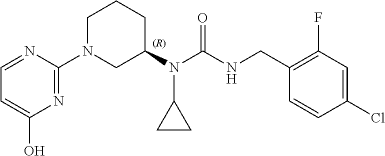

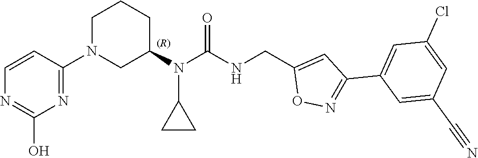

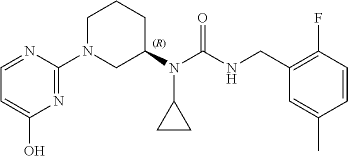

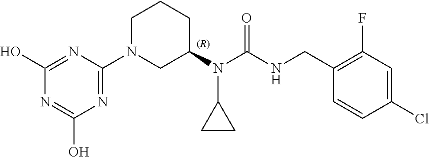

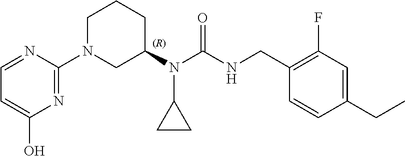

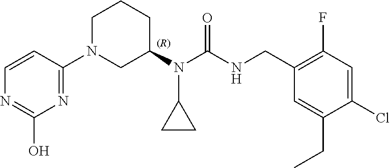

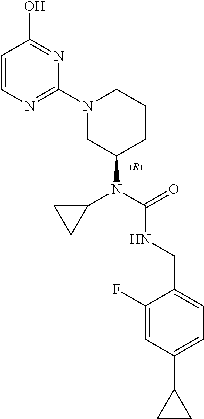

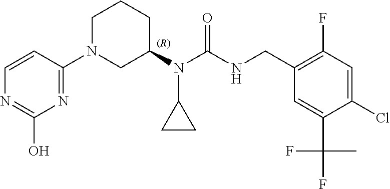

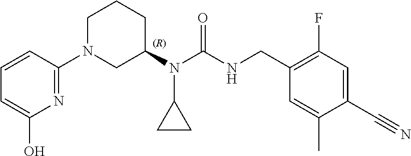

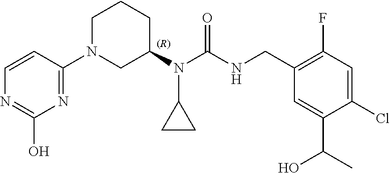

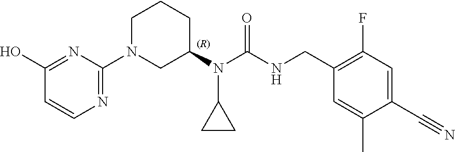

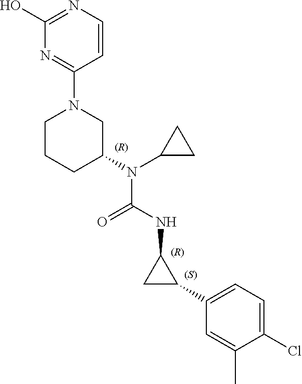

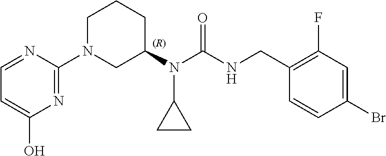

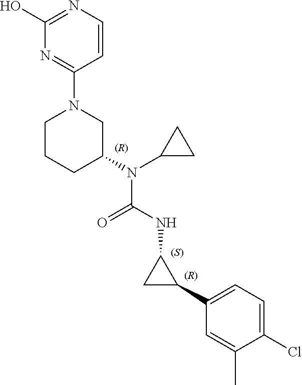

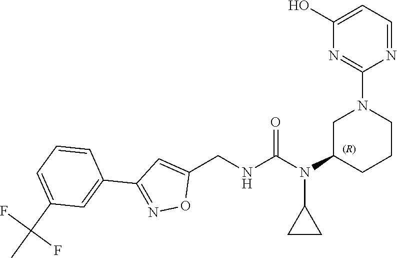

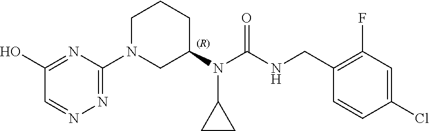

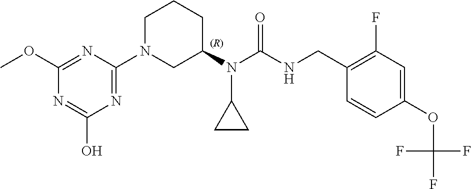

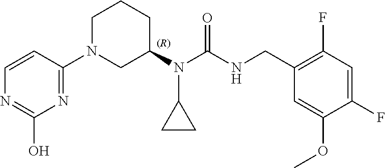

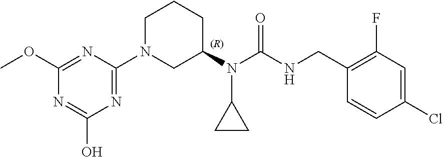

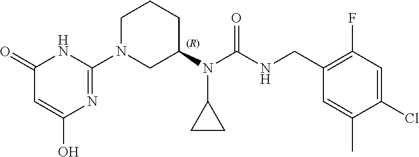

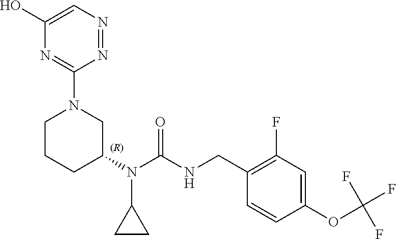

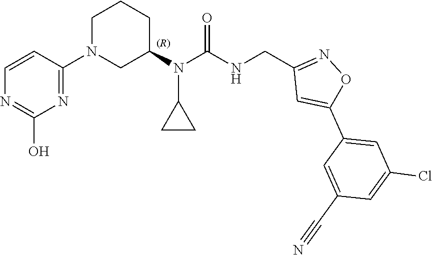

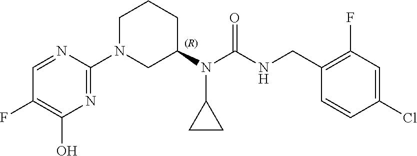

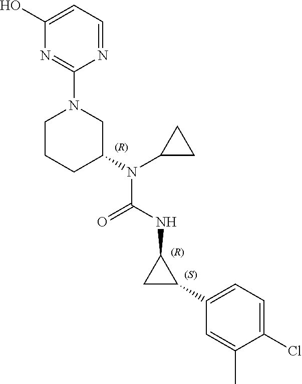

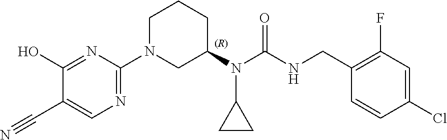

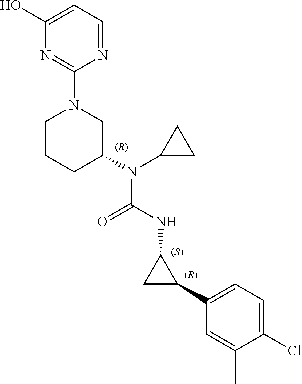

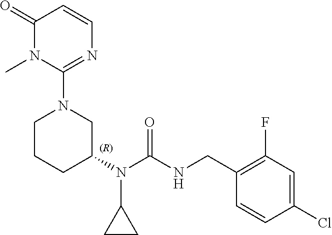

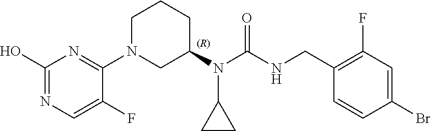

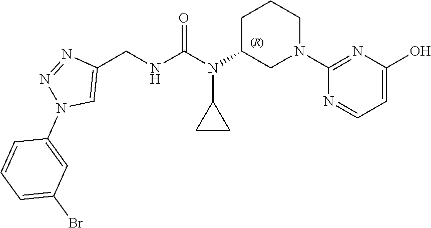

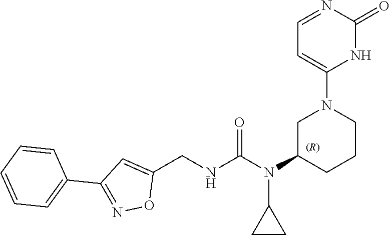

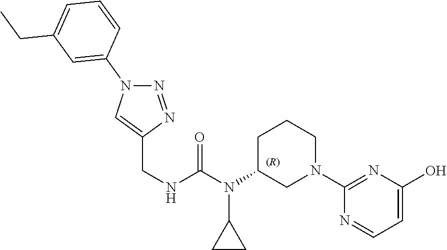

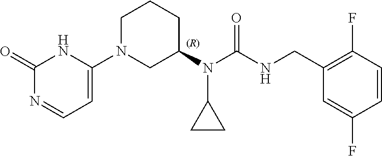

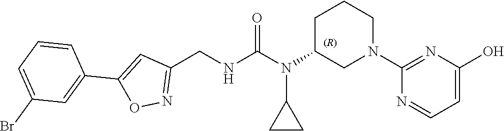

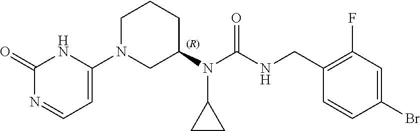

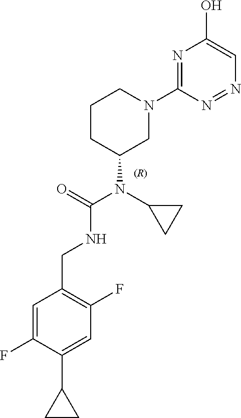

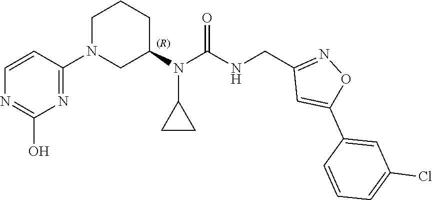

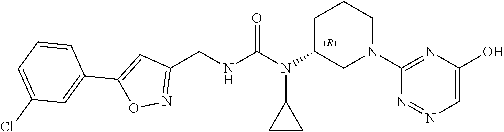

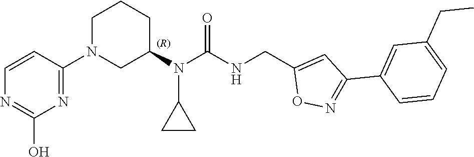

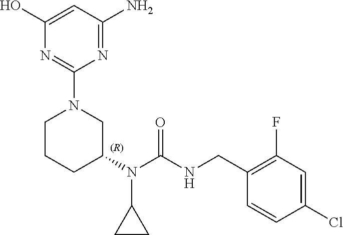

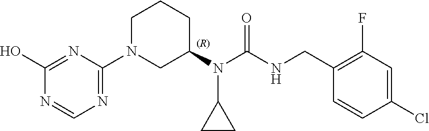

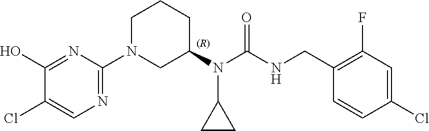

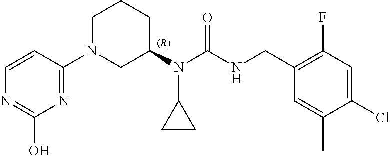

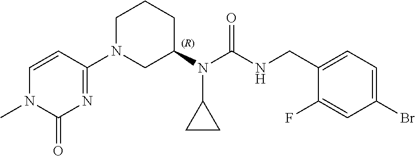

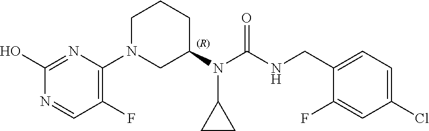

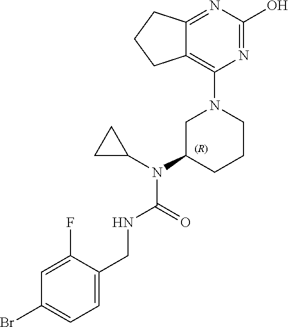

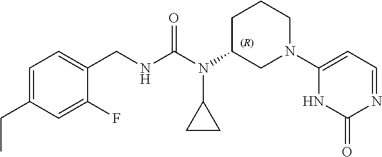

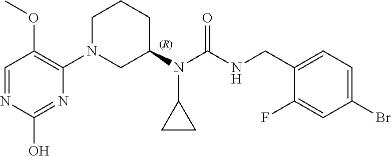

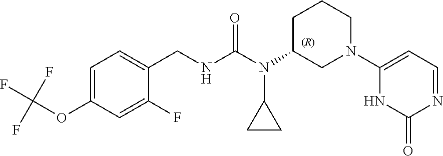

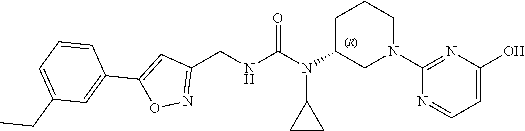

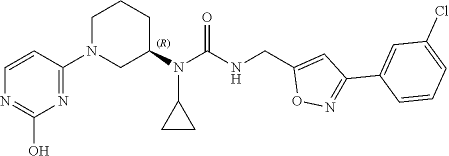

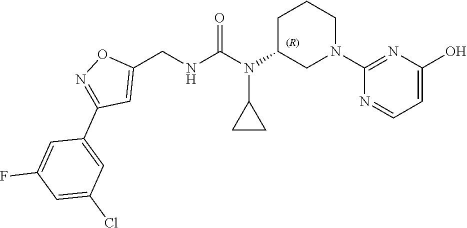

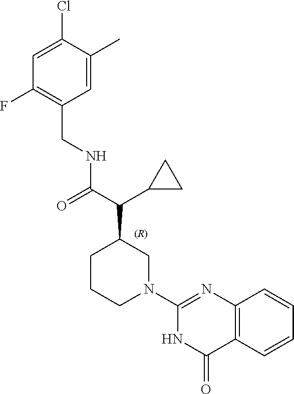

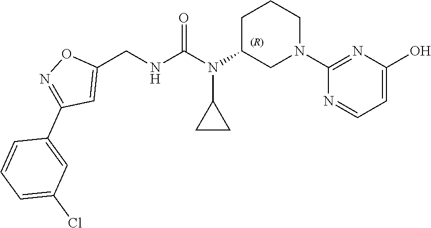

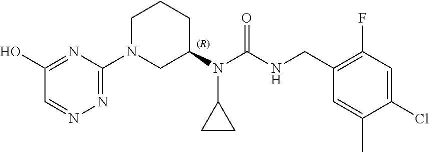

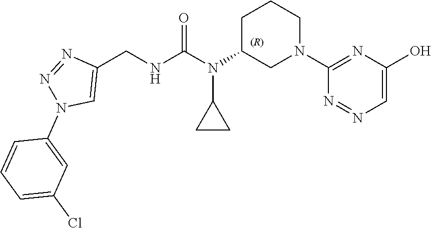

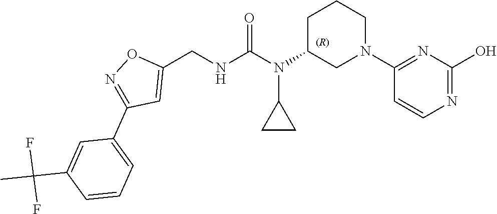

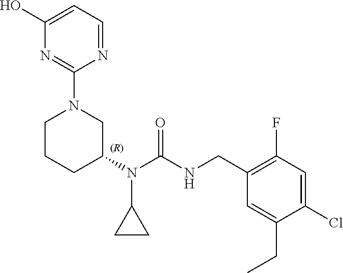

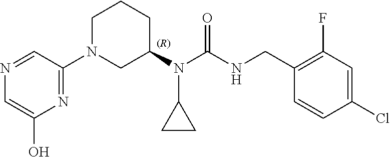

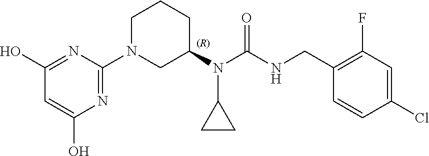

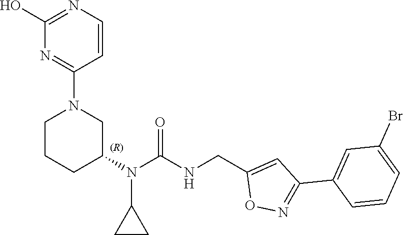

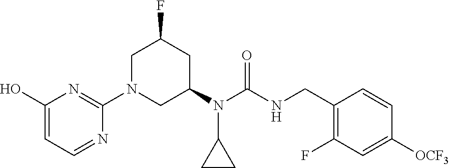 [SMILES: O=C(NCc1ccc(OC(F)(F)F)cc1F)N(C1CC1)[C@@H]1C[C@H](F)CN(c2nccc(O)n2)C1]

In some embodiments, the compounds are atropisomers. Additionally, unless otherwise stated, structures depicted herein are also meant to include compounds that differ only in the presence of one or more isotopically enriched atoms. For example, compounds produced by the replacement of a hydrogen with deuterium or tritium, or of a carbon with a ¹³- or ¹⁴C-enriched carbon are within the scope of this invention. Such compounds are useful, for example, as analytical tools, as probes in biological assays, or as therapeutic agents in accordance with the present invention. For example, in the case of variable R₁, the (C₁-C₄)alkyl or the —O—(C₁-C₄)alkyl can be suitably deuterated (e.g., —CD₃, —OCD₃).

Any compound of the invention can also be radiolabed for the preparation of a radiopharmaceutical agent.

### Methods of Treatment

One aspect of the invention provides compounds, compositions, and methods useful for treating or preventing a disease or disorder associated with abnormal levels of amino acids by modulation of SLC6A19 transport.

Another aspect of the invention relates to methods of modulating SLC6A19 transport in a subject in need thereof comprising administering to the subject an effective amount of a compound of Formula (I).

Another aspect of the invention relates to methods of treating or preventing a disease or disorder associated with a genetic defect in phenylalanine hydroxylase in a subject in need thereof comprising administering to the subject an effective amount of a compound of Formula (I).

In some embodiments, the invention relates to methods of treating or preventing phenylketonuria in a subject in need thereof comprising administering to the subject an effective amount of a compound of Formula (I).

In some embodiments, the invention relates to methods of treating or preventing hyperphenylalaninemia in a subject in need thereof comprising administering to the subject an effective amount of a compound of Formula (I).

In some embodiments, the compound reduces systemic phenylalanine levels in the subject.

In some embodiments, the invention relates to methods of treating or preventing tyrosinemia (Type I, II, or III) in a subject in need thereof comprising administering to the subject an effective amount of a compound of Formula (I).

In some embodiments, the compound reduces systemic glycine levels in the subject.

In some embodiments, the invention relates to methods of treating or preventing isovaleric academia, methylmalonic academia, propionic academia, maple syrup urine disease, DNAJC12 deficiency, urea cycle disorders, or hyperammonemia in a subject in need thereof comprising administering to the subject an effective amount of a compound of Formula (I).

In some embodiments of any one of the disclosed methods, the compound modulates SLC6A19 in the subject.

In some embodiments of any one of the disclosed methods, the compound inhibits SLC6A19 in the subject.

In some embodiments of any one of the disclosed methods, the compound modulates SLC6A19 transport in the subject.

In some embodiments of any one of the disclosed methods, the compound inhibits SLC6A19 transport in the subject.

In some embodiments, the compound reduces systemic levels of an amino acid in the subject.

In some embodiments of any one of the disclosed methods, wherein the subject is a mammal. In some embodiments of any one of the disclosed methods, the mammal is a human.

In some embodiments of any one of the disclosed methods, the compound of Formula (I) is defined as:

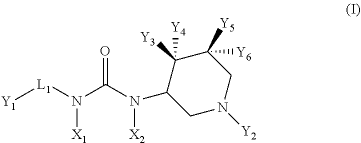

wherein:
L₁ is absent or selected from -alkyl-, -cycloalkyl-, and -heteroaryl-CH₂—;
X₁ and X₂ are independently selected from —H, alkyl, cycloalkyl, and alkyl-cycloalkyl; provided that X₁ and X₂ are not both —H;
Y₁ is optionally substituted aryl; and
Y₂ is selected from optionally substituted pyridonyl, optionally substituted pyrimidinoyl, optionally substituted pyrazinonyl, optionally substituted triazinonyl, and optionally substituted quinazolinonyl;
Y₃, Y₄, Y₅, and Y₆ are independently selected from —H and halide;
or a pharmaceutically acceptable salt thereof.

In some embodiments of any one of the disclosed methods, the compound of Formula (I) is defined as:

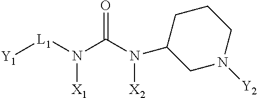

wherein:
L₁ is absent or selected from -alkyl-, -cycloalkyl-, and -heteroaryl-CH₂—;
X₁ and X₂ are independently selected from —H, alkyl, cycloalkyl, and alkyl-cycloalkyl; provided that X₁ and X₂ are not both —H;
Y₁ is optionally substituted aryl; and
Y₂ is selected from optionally substituted pyridonyl, optionally substituted pyrimidinoyl, optionally substituted pyrazinonyl, optionally substituted triazinonyl, and optionally substituted quinazolinonyl;
or a pharmaceutically acceptable salt thereof.

### Pharmaceutical Compositions, Routes of Administration, and Dosing

In certain embodiments, the invention is directed to a pharmaceutical composition, comprising a compound of the invention and a pharmaceutically acceptable carrier. In certain embodiments, the pharmaceutical composition comprises a plurality of compounds of the invention and a pharmaceutically acceptable carrier.

In certain embodiments, a pharmaceutical composition of the invention further comprises at least one additional pharmaceutically active agent other than a compound of the invention. The at least one additional pharmaceutically active agent can be an agent useful in the treatment of ischemia-reperfusion injury.

Pharmaceutical compositions of the invention can be prepared by combining one or more compounds of the invention with a pharmaceutically acceptable carrier and, optionally, one or more additional pharmaceutically active agents.

As stated above, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the invention being administered, the size of the subject, or the severity of the disease or condition. One of ordinary skill in the art can empirically determine the effective amount of a particular compound of the invention and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

In certain embodiments, intravenous administration of a compound may typically be from 0.1 mg/kg/day to 20 mg/kg/day. In one embodiment, intravenous administration of a compound may typically be from 0.1 mg/kg/day to 2 mg/kg/day. In one embodiment, intravenous administration of a compound may typically be from 0.5 mg/kg/day to 5 mg/kg/day. In one embodiment, intravenous administration of a compound may typically be from 1 mg/kg/day to 20 mg/kg/day. In one embodiment, intravenous administration of a compound may typically be from 1 mg/kg/day to 10 mg/kg/day.

Generally, daily oral doses of a compound will be, for human subjects, from about 0.01 milligrams/kg per day to 1000 milligrams/kg per day. It is expected that oral doses in the range of 0.5 to 50 milligrams/kg, in one or more administrations per day, will yield therapeutic results. Dosage may be adjusted appropriately to achieve desired drug levels, local or systemic, depending upon the mode of administration. For example, it is expected that intravenous administration would be from one order to several orders of magnitude lower dose per day. In the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The formulations of the invention can be administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the compound can be administered to a subject by any mode that delivers the compound to the desired surface. Administering a pharmaceutical composition may be accomplished by any means known to the skilled artisan. Routes of administration include but are not limited to intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound of the invention can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers, e.g., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of acid hydrolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Abuchowski and Davis, “Soluble Polymer-Enzyme Adducts”, In: Enzymes as Drugs, Hocenberg and Roberts, eds., Wiley-Interscience, New York, N.Y., pp. 367-383 (1981); Newmark et al., J Appl Biochem 4:185-9 (1982). Other polymers that could be used are poly-1,3-dioxolane and poly-1,3,6-tioxocane. For pharmaceutical usage, as indicated above, polyethylene glycol moieties are suitable.

For the component (or derivative) the location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the compound of the invention (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic (e.g., powder); for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi-particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the compound of the invention (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, α-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents which can be used and can include benzalkonium chloride and benzethonium chloride. Potential non-ionic detergents that could be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound of the invention or derivative either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For topical administration, the compound may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

Also contemplated herein is pulmonary delivery of the compounds disclosed herein (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (α1-antitrypsin); Smith et al., 1989, J Clin Invest 84:1145-1146 (α-1-proteinase); Oswein et al., 1990, “Aerosolization of Proteins”, Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated by reference). A method and composition for pulmonary delivery of drugs for systemic effect is described in U.S. Pat. No. 5,451,569 (incorporated by reference), issued Sep. 19, 1995 to Wong et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of the compounds of the invention. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated. Chemically modified compound of the invention may also be prepared in different formulations depending on the type of chemical modification or the type of device employed.

Formulations suitable for use with a nebulizer, either jet or ultrasonic, will typically comprise a compound of the invention (or derivative) dissolved in water at a concentration of about 0.1 to 25 mg of biologically active compound of the invention per mL of solution. The formulation may also include a buffer and a simple sugar (e.g., for inhibitor stabilization and regulation of osmotic pressure). The nebulizer formulation may also contain a surfactant, to reduce or prevent surface induced aggregation of the compound of the invention caused by atomization of the solution in forming the aerosol.

Formulations for use with a metered-dose inhaler device will generally comprise a finely divided powder containing the compound of the invention (or derivative) suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing compound of the invention (or derivative) and may also include a bulking agent, such as lactose, sorbitol, sucrose, or mannitol in amounts which facilitate dispersal of the powder from the device, e.g., 50 to 90% by weight of the formulation. The compound of the invention (or derivative) should advantageously be prepared in particulate form with an average particle size of less than 10 micrometers (μm), most preferably 0.5 to 5 μm, for most effective delivery to the deep lung.

Nasal delivery of a pharmaceutical composition of the present invention is also contemplated. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available. Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethylcellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described above, a compound may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-33 (1990).

The compound of the invention and optionally other therapeutics may be administered per se (neat) or in the form of a pharmaceutically acceptable salt or cocrystal. When used in medicine the salts or cocrystals should be pharmaceutically acceptable, but non-pharmaceutically acceptable salts or cocrystals may conveniently be used to prepare pharmaceutically acceptable salts or cocrystals thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions of the invention contain an effective amount of a compound as described herein and optionally therapeutic agents included in a pharmaceutically acceptable carrier. The term “pharmaceutically acceptable carrier” means one or more compatible solid or liquid filler, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agent(s), including specifically but not limited to a compound of the invention, may be provided in particles. Particles as used herein means nanoparticles or microparticles (or in some instances larger particles) which can consist in whole or in part of the compound of the invention or the other therapeutic agent(s) as described herein. The particles may contain the therapeutic agent(s) in a core surrounded by a coating, including, but not limited to, an enteric coating. The therapeutic agent(s) also may be dispersed throughout the particles. The therapeutic agent(s) also may be adsorbed into the particles. The particles may be of any order release kinetics, including zero-order release, first-order release, second-order release, delayed release, sustained release, immediate release, and any combination thereof, etc. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules which contain the compound of the invention in a solution or in a semi-solid state. The particles may be of virtually any shape.

Both non-biodegradable and biodegradable polymeric materials can be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels described in Sawhney H S et al. (1993) Macromolecules 26:581-7, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. The term “controlled release” is intended to refer to any drug-containing formulation in which the manner and profile of drug release from the formulation are controlled. This refers to immediate as well as non-immediate release formulations, with non-immediate release formulations including but not limited to sustained release and delayed release formulations. The term “sustained release” (also referred to as “extended release”) is used in its conventional sense to refer to a drug formulation that provides for gradual release of a drug over an extended period of time, and that preferably, although not necessarily, results in substantially constant blood levels of a drug over an extended time period. The term “delayed release” is used in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. “Long-term” release, as used herein, means that the implant is constructed and arranged to deliver therapeutic levels of the active ingredient for at least 7 days, and preferably 30-60 days. Long-term sustained release implants are well-known to those of ordinary skill in the art and include some of the release systems described above.

It will be understood by one of ordinary skill in the relevant arts that other suitable modifications and adaptations to the compositions and methods described herein are readily apparent from the description of the invention contained herein in view of information known to the ordinarily skilled artisan, and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

The invention is further described in the following examples, which do not limit the scope of the invention described in the claims.

### SLC6A19 Isoleucine Transport Assay

### Cell Line Generation and Maintenance

The Flp-In™ T-REx™ 293 cell line was purchased from Thermo Fisher Scientific. The line was used to generate a stable cell line inducibly expressing human SLC6A19 with a C-terminal V5 tag and stably expressing human TMEM27 (also known as Collectrin) with a C-terminal myc-DDK tag. The stable cell line was generated by transfecting SLC6A19- and TMEM27-encoding plasmids using standard protocols, followed by antibiotic selection. Stable cells were maintained in DMEM/F12 supplemented with Glutamax, 10% fetal bovine serum, 100 U/mL penicillin, 100 μg/mL streptomycin, 200 μg/mL hygromycin, 10 μg/mL blasticidin and 300 μg/mL neomycin (Thermo Fisher).

### Assay: Isoleucine Transport Assay in 96-Well Format

Stable cell lines were seeded at a density of 35,000 cells per well in a poly-D-lysine coated 96-well cell culture-treated plate on day 0. On day 1 the expression of SLC6A19 was induced by dispensing tetracycline at a final concentration of 1 μg/mL using a Tecan D300e digital dispenser. On day 2 the transport assay was run. Media was removed from the plate using the GentleSpin setting of a Centrifugal Blue Washer (Blue Cat Bio) and cells were washed with 175 uL live cell imaging solution (Thermo Fisher) using the Blue Washer. Following washing, cells were treated with 70 μL of either DMSO, positive control or compound, diluted in Krebs buffer (140 mM NaCl, 4.7 mM KCl, 2.5 mM CaCl₂), 1.2 mM MgCl₂, 11 mM HEPES, 10 mM Glucose, pH 7.4) at room temperature. After 20-60 minutes 30 μL of a 3.3 mM solution of 13C6,15N-L-isoleucine (Cambridge Isotope Laboratories) was added. After 20 min incubation with the isoleucine substrate at room temperature cells were washed with 175 uL live cell imaging solution using the Blue Washer. Cells were then lysed in 150 μL of 15 uM D-Leucine-d10 (CDN Isotopes) in ultrapure water. Plates were put on a shaker at 700 rpm for a minimum of 40 minutes to facilitate lysis. Following lysis, a standard dilution curve of 13C6,15N-L-isoleucine was added to wells containing lysates of untreated cells. Plates were returned to the shaker for a minimum of 2 minutes to ensure proper mixing of the standard curve. Plates were then centrifuged for 5 min at 4,000 rpm to pellet cellular debris and precipitate. Supernatants were diluted 1:10 in acetonitrile+0.1% formic acid in polypropylene plates.

### Assay: Isoleucine Transport Assay in 384-Well Format

On day 0, stable cell lines were seeded at a density of 20,000 cells per well in a poly-D-lysine coated 384-well cell culture-treated plate in media containing 1 μg/mL tetracycline using a Viaflo 384-well pipette. Transport assays were run the following day (day 1). Media was removed from the plate using the GentleSpin setting of a Centrifugal Blue Washer (Blue Cat Bio) and cells were washed with 80 uL live cell imaging solution (Thermo Fisher) using the Blue Washer. Following washing, cells were treated with 20 μL of either DMSO, positive control or compound, diluted in Krebs buffer (140 mM NaCl, 4.7 mM KCl, 2.5 mM CaCl₂), 1.2 mM MgCl₂, 11 mM HEPES, 10 mM Glucose, pH 7.4) using a TECAN liquid handler. After 20-60 minutes incubation at room temperature 8.6 uL of a 3.3 mM solution of 13C6,15N-L-isoleucine (Cambridge Isotope Laboratories) was added. After 20 min incubation with the isoleucine substrate at room temperature cells were washed with 80 uL live cell imaging solution using the Blue Washer. Cells were then lysed in 80 μL of 15 uM D-Leucine-d10 (CDN Isotopes) in ultrapure water. Plates were put on a shaker at 700 rpm for a minimum of 2 hours to facilitate lysis. Following lysis, a standard dilution curve of 13C6,15N-L-isoleucine was added to wells containing lysates of untreated cells. Plates were returned to the shaker for a minimum of 5 minutes to ensure proper mixing of the standard curve. Plates were then centrifuged for 10 min at 4,000 rpm to pellet cellular debris and precipitate. Supernatants were diluted 1:10 in acetonitrile+0.1% formic acid in polypropylene plates.

13C6,15N-L-isoleucine analysis was performed using a RapidFire365-QTOF 6545 (Agilent). Quantitative sample analysis utilizes automated solid-phase extraction (HILIC H6 cartridge) prior to mass spec injection. Samples were loaded using 95% acetonitrile, 0.1% formic acid and eluted from the cartridge with 5% acetonitrile, 0.1% formic acid directly for ESI-MS (electrospray ionization) analysis. Quantification of the analytes were performed using Agilent Masshunter Quant software from the high-resolution full scan data.

### General Procedures

### General Procedure 1: Synthesis of 3-aril 5-aminomethyl isoxazoles

3-aryl 5-aminomethyl isoxazoles used or referenced below were prepared using an appropriate modification of the following procedure and the appropriate starting arenes.

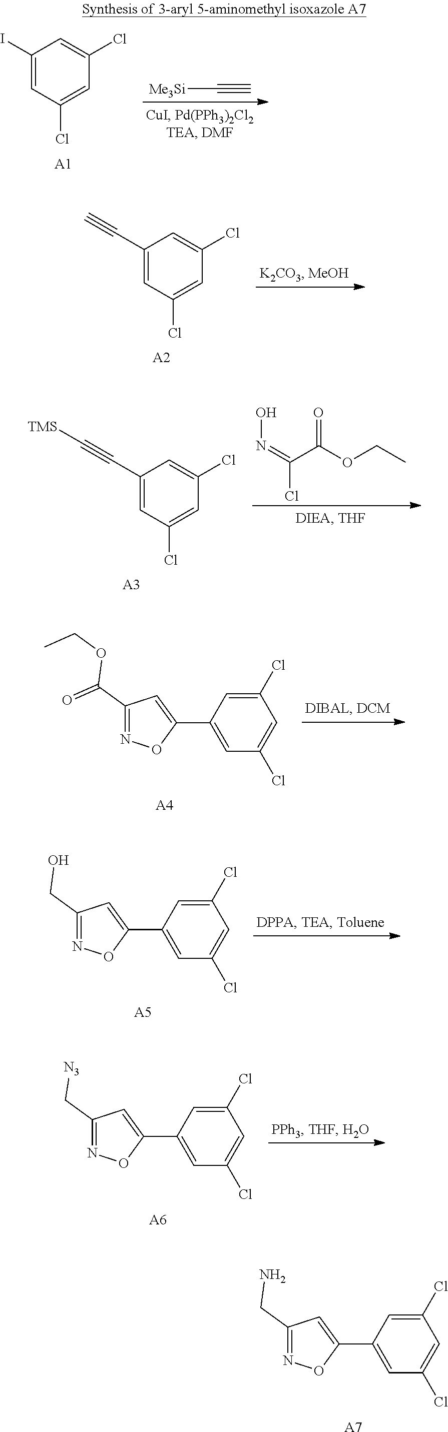

### Step 1: Synthesis of Compound A2

To a mixture of compound A1 (800 mg, 2.931 mmol) and TEA (888 mg, 8.8 mmol) in DMF (15 mL) was added trimethylsilylacetylene (0.42 mL, 2.931 mmol), CuI (56 mg, 0.293 mmol) and Pd(PPh₃)₂Cl₂ (215 mg, 0.293 mmol). The resulting mixture was stirred at 50° C. for 16 hrs under N₂ atmosphere. Then the mixture was diluted with EtOAc (50 mL) and filtered, the filtrate was washed with saturated NH₄Cl solution (50 mL×2), water (50 mL) and brine (50 mL). The organic layer was separated, dried over anhydrous Na₂SO₄ and concentrated to dryness. The residue was purified by column chromatography on silica gel (eluted with PE:EtOAc=100:0 to 95:5) to afford compound A2 (650 mg, 91.17% yield) as light-yellow solid.

### Step 2: Synthesis of Compound A3

To a mixture of compound A2 (650 mg, 2.673 mmol) in MeOH (10 mL) was added K₂CO₃ (1.85 g, 13.363 mmol). The resulting mixture was stirred at room temperature for 30 mins. Then the mixture was filtered and the filtrate was concentrated under reduced pressure to give crude compound A3 (350 mg, 76.57% yield) as yellow oil without further purification. 1H NMR (400 MHz, MeOD) δ 7.47 (t, J=1.8 Hz, 1H), 7.42 (d, J=1.8 Hz, 2H), 3.74 (s, 1H).

### Step 3: Synthesis of Compound A4

To a mixture of compound A3 (350 mg, 2.046 mmol) and DIEA (532 mg, 4.093 mmol) in THE (12 mL) was added ethyl 2-chloro-2-(hydroxyimino)acetate (465 mg, 3.070 mmol) slowly at 0° C. under N₂ atmosphere. The resulting mixture was stirred at room temperature for 18 hrs. Then the mixture was diluted with EtOAc (40 mL) and washed with water and brine, the organic layer was separated, dried over anhydrous Na₂SO₄ and concentrated to dryness. The crude product was purified by column chromatography on silica gel (eluted with PE:EA=100:0 to 85:15) to afford compound A4 (300 mg, 51.24% yield) as yellow oil. LC/MS (ESI) m/z: 286 (M+H)⁺.

### Step 4: Synthesis of Compound A5

To a solution of compound A4 (300 mg, 1.049 mmol) in anhydrous DCM (12 mL) was added DIBAL (3.2 mL, 1 M in hexane) at −30° C. under N₂ atmosphere. The resulting mixture was allowed to warm to room temperature for 2 hrs. Then the mixture was poured into iced NH₄Cl solution (30 mL) and extracted with EtOAc (30 mL). The organic layer was separated, dried over anhydrous Na₂SO₄ and concentrated to dryness. The residue was purified by column chromatography on silica gel (eluted with DCM:MeOH=100:0 to 95:5) to afford compound A5 (180 mg, 70.33% yield) as light-yellow solid. LC/MS (ESI) m/z: 244 (M+H)⁺.

### Step 5: Synthesis of Compound A6

To a solution of compound A5 (180 mg, 0.737 mmol) in toluene (12 mL) was added DPPA (264 mg, 0.959 mmol) and DBU (1.3 mL, 8.112 mmol). The resulting mixture was stirred at room temperature for 18 hrs under N₂ atmosphere. Then the mixture was diluted with EtOAc (30 mL) and washed with water (40 mL) and brine (40 mL). The organic layer was separated, dried over anhydrous Na₂SO₄ and concentrated under reduced pressure to give crude compound A6 (197 mg, 99.27% yield) as colorless oil which was used in the next step directly without further purification. LC/MS (ESI) m/z: 269 (M+H)⁺.

### Step 6: Synthesis of Compound A7

To a solution of compound A6 (197 mg, 0.732 mmol) in THE (8 mL) and H₂O (2 mL) was added PPh₃ (384 mg, 1.464 mmol). The resulting mixture was stirred at room temperature for 18 hrs under N₂ atmosphere. Then the mixture was acidified with 1N HCl solution to pH=6 and the resulting mixture was extracted with methyl tert-butyl ether (40 mL). The aqueous layer was separated, basified with saturated NaHCO₃ solution to pH=8 and then extracted with EtOAc (30 mL) twice. The combined organic layers were washed with water (50 mL) and brine (50 mL), dried over anhydrous Na₂SO₄ and concentrated to dryness. The residue was purified by column chromatography on silica gel (eluted with DCM:MeOH=100:0 to 95:5) to give compound A7 (150 mg, 84.29% yield) as colorless oil. LC/MS (ESI) m/z: 243 (M+H)+.

### General Procedure 2: Synthesis of 3-aminomethyl 5-aryl isoxazoles

3-aryl 5-aminomethyl isoxazoles used or referenced below were prepared using an appropriate modification of the following procedure and the appropriate starting arenes.

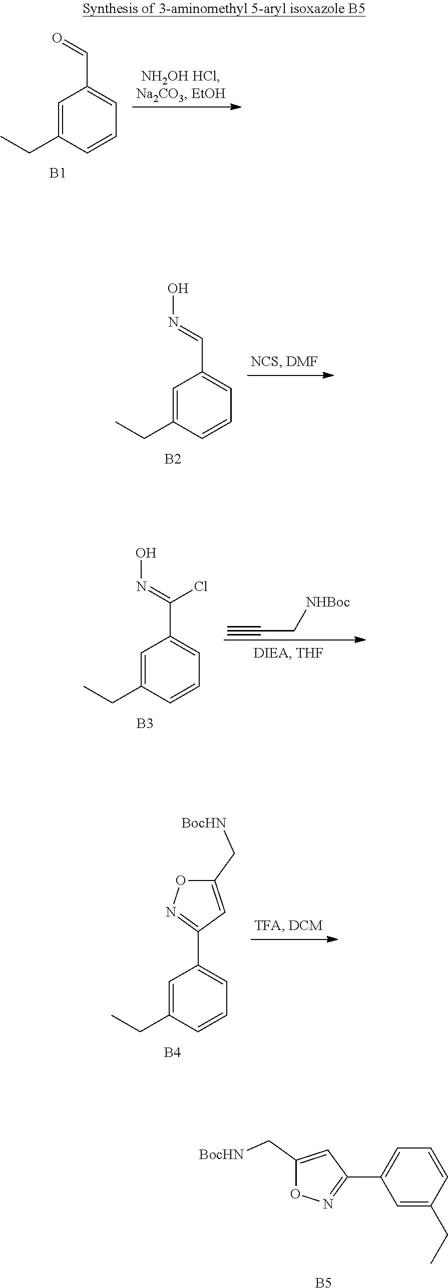

### Step 1: Synthesis of Compound B2

To a mixture of compound B1 (800 mg, 5.962 mmol) and disodium carbonate (2.53 g, 23.849 mmol) in EtOH (20 mL) was added NH₂OH—HCl (1.16 g, 7.751 mmol). The resulting mixture was stirred at 40° C. for 30 mins. Then the mixture was concentrated under reduced pressure to dryness. The residue was dissolved with EtOAc (40 mL) and washed with water (40 mL) and brine (40 mL). The organic layer was separated, dried over anhydrous Na₂SO₄ and concentrated under reduced pressure to give crude compound B2 (880 mg, 98.9% yield) as white solid which was used in the next step directly without further purification. LC/MS (ESI) m/z: 150 (M+H)⁺.

### Step 2: Synthesis of Compound 3

To a solution of compound B2 (880 mg, 5.899 mmol) in DMF (15 mL) was added NCS (1.18 g, 8.848 mmol) and the resulting mixture was stirred at 40° C. for 3 hrs. Then the mixture was diluted with saturated NH₄Cl solution (40 mL) and extracted with EtOAc (40 mL×2). The combined organic layers were washed with water (50 mL) and brine (50 mL), dried over anhydrous Na₂SO₄ and concentrated under reduced pressure to give crude compound B3 (1.0 g, 92.32% yield) as white solid which was used in the next step directly without further purification.

### Step 3: Synthesis of Compound B4

To a mixture of compound 3 (1 g, 5.445 mmol) and DIEA (2.12 g, 16.336 mmol) in anhydrous THE (18 mL) was added tert-butyl N-(prop-2-yn-1-yl)carbamate (0.85 g, 5.445 mmol) at 0° C. dropwise. The resulting mixture was stirred at 40° C. for 2 hrs. Then the mixture was concentrated to dryness under reduced pressure and the residue was purified by column chromatography on silica gel (eluted with PE:EtOAc=100:0 to 5:1) to give compound 4 (1.2 g, 72.88% yield) as white solid. LC/MS (ESI) m/z: 303 (M+H)⁺.

### Step 4: Synthesis of Compound B5

To a solution of compound B4 (400 mg, 1.323 mmol) in DCM (5 mL) was added TFA (1 mL) dropwise at 0° C. The resulting mixture was stirred at r.t. for 2 hrs. Then the mixture was concentrated under reduced pressure to dryness. The residue was dissolved in EtOAc (30 mL) and washed with saturated NaHCO₃ solution (60 mL). The organic layer was separated, dried over anhydrous Na₂SO₄ and concentrated under reduced pressure to give crude compound B5 (220 mg, 82.39% yield) as colorless oil which was used in the next step directly without further purification. LC/MS (ESI) m/z: 203 (M+H)⁺.

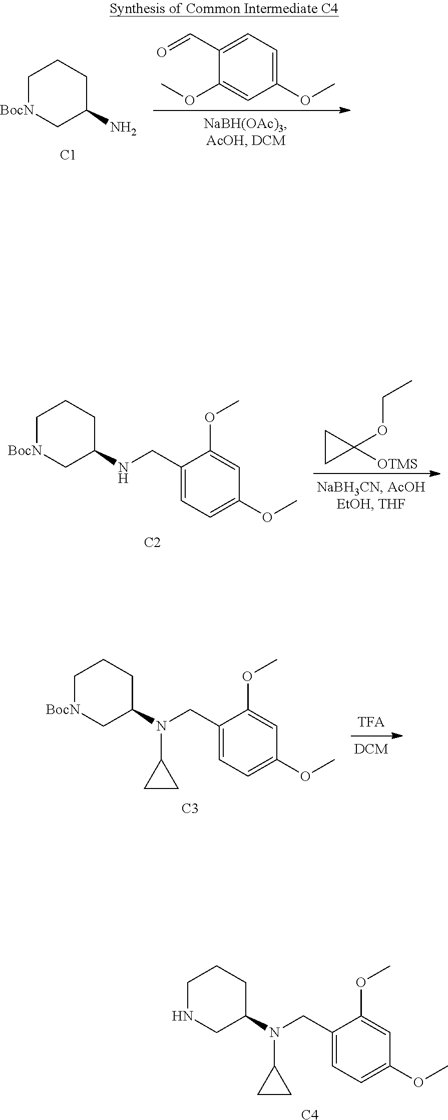

### Step 1: Synthesis of Compound C2

To a mixture of compound C1 (310 g, 1547.83 mmol) in DCM (7000 mL) was added AcOH (278.48 g, 4643.49 mmol) and 2,4-dimethoxybenzaldehyde (219.6 mL, 1547-95 mmol) at room temperature under N₂ atmosphere. After stirring at 0° C. for 3 hrs, sodium borohydride (984.3 g, 4643.73 mmol) was added into the above mixture in portions during 2 hrs. The resulting mixture was stirred at room temperature for 16 hrs. Then the mixture was concentrated under reduced pressure to dryness. The residue was diluted with DCM (5000 mL) and washed with water (5000 mL) and brine (5000 mL). The organic layer was separated, dried over anhydrous Na₂SO₄ and concentrated to dryness. The crude product was purified by column chromatography on silica gel (eluted with DCM:MeOH=100:0 to 95:5) to afford compound C₂ (515 g, 94.90% yield) as light-yellow oil. LC/MS (ESI) m/z: 351 (M+H)⁺. ¹H NMR (400 MHz, MeOD) δ 7.24 (d, J=8.3 Hz, 1H), 6.60 (d, J=1.9 Hz, 1H), 6.54 (dd, J=8.3, 2.2 Hz, 1H), 4.13 (s, 1H), 4.01 (s, 2H), 3.88 (s, 3H), 3.81 (s, 4H), 2.93 (d, J=11.0 Hz, 3H), 2.14-2.05 (m, 1H), 1.91 (s, 2H), 1.79-1.72 (m, 1H), 1.46 (s, 9H).

### Step 2: Synthesis of Compound C3

To a mixture of C2 (515 g, 1468.78 mmol), AcOH (1322.5 g, 22042 mmol) and (1-ethoxycyclopropoxy)trimethylsilane (512.0 g, 2937.56 mmol) in EtOH (2000 mL) and THE (8000 mL) was added sodium cyanoborohydride (323.4 g, 5141.76 mmol) in portions at 0° C. under N₂ atmosphere during 2 hrs. The resulting mixture was stirred at 80° C. for 8 hrs. Then the mixture was concentrated under reduced pressure to dryness. The residue was diluted with DCM (5000 mL) and washed with water (5000 mL) and brine (5000 mL). The organic layer was separated, dried over anhydrous Na₂SO₄ and concentrated to dryness. The crude product was purified by column chromatography on silica gel (eluted with DCM:MeOH=100:0 to 96:4) to afford compound C₃ (406.5 g, 70.8% yield) as light-yellow oil. LC/MS (ESI) m/z: 391 (M+H)⁺. ¹H NMR (400 MHz, MeOD) δ 7.27 (d, J=8.3 Hz, 1H), 6.59-6.48 (m, 2H), 4.38 (s, 1H), 4.12 (s, 2H), 4.03-3.96 (m, 1H), 3.83 (s, 3H), 3.80 (s, 3H), 3.00-2.85 (m, 2H), 2.78-2.63 (m, 1H), 2.50-2.30 (m, 1H), 2.25-2.15 (m, 1H), 1.98 (s, 1H), 1.85-1.69 (m, 2H), 1.43 (s, 9H), 0.68 (s, 2H) 0.53 (s, 2H).

### Step 3: Synthesis of Compound C4

To a solution of C3 (406.5 g, 1039.08 mmol) in DCM (4000 mL) at 0° C. under N₂ atmosphere was added TFA (1000 mL) drop-wise within 2 hrs. The resulting mixture was stirred at room temperature for 3 hrs. Then the mixture was concentrated under reduced pressure to dryness. The residue was diluted with DCM (2000 mL) and 10% Na₂CO₃ solution was added progressively to adjust pH=8. Then the aqueous layer was extracted with DCM (1500 mL×4). The combined organic layers were washed with water and brine, dried over anhydrous Na₂SO₄ and concentrated to give crude C₄ (275.0 g, 90.9% yield) as yellow oil without further purification. LC/MS (ESI) m/z: 291 (M+H)⁺. ¹H NMR (400 MHz, MeOD) δ 7.18 (d, J=8.3 Hz, 1H), 6.51-6.43 (m, 2H), 3.80-3.76 (m, 8H), 3.19 (d, J=8.2 Hz, 1H), 3.08-3.02 (m, 1H), 2.82-2.72 (m, 2H), 2.65-2.54 (m, 1H), 2.13-2.06 (m, 1H), 2.03-1.96 (m, 1H), 1.89-1.82 (m, 1H), 1.69-1.47 (m, 2H), 0.53-0.44 (m, 2H), 0.33 (d, J=2.6 Hz, 2H).

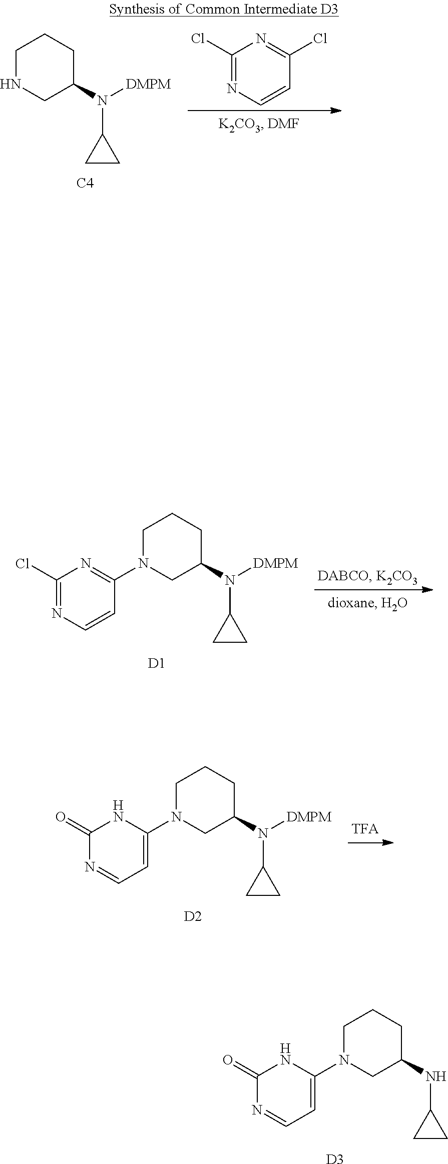

### Step 1: Synthesis of Compound D1

To a solution of compound C₄ (100 mg, 0.344 mmol) in DMF (8 mL) was added 3,6-dichloropyridazine (190 mg, 1.278 mmol) and K₂CO₃ (142 mg, 1.033 mmol). The resulting mixture was stirred at 80° C. for 16 hrs under N₂ atmosphere. After cooling, the mixture was diluted with EtOAc (40 mL) and washed with saturated NH₄Cl solution (30 mL×3) and brine (30 mL). The organic layer was separated, dried over anhydrous Na₂SO₄ and concentrated to dryness. The residue was purified by column chromatography on silica gel (eluted with PE:EtOAc=100:0 to 2:1) to give compound D1 (120 mg, 86.5% yield) as colorless oil. LC/MS (ESI) m/z: 403 (M+H)⁺.

### Step 2: Synthesis of Compound D2

To a solution of compound D1 (120 mg, 0.298 mmol) in dioxane (2 mL) and H₂O (2 mL) was added DABCO (50 mg, 0.447 mmol) and K₂CO₃ (123 mg, 0.893 mmol). The resulting mixture was stirred at 80° C. for 16 hrs under N₂ atmosphere. After cooling, the mixture was diluted with EtOAc (40 mL) and washed with water (30 mL) and brine (30 mL). The organic layer was separated, dried over anhydrous Na₂SO₄ and filtered. The filtrate was evaporated to dryness. The residue was purified by column chromatography on silica gel (eluted with DCM:MeOH=100:0 to 20:1) to give compound D2 (90 mg, 78.6% yield) as colorless oil. LC/MS (ESI) m/z: 385 (M+H)⁺.

### Step 3: Synthesis of Compound D3

Compound D2 (90 mg, 0.234 mmol) was added into TFA (5 mL) in portions at 0° C. under N₂ atmosphere. Then the resulting mixture was stirred at 80° C. for 4 hrs. After cooling to room temperature, the mixture was concentrated under reduced pressure to give crude compound D3 (54 mg, 98.4% yield) as purple oil which was used at the next step directly without further purification. LC/MS (ESI) m/z: 235 (M+H)⁺.

### Examples 1-11

The compounds in the table below were prepared from the appropriate starting materials, prepared as described above or commercially available, using an appropriate modification of General Procedure 3 and (R)-6-(3-(cyclopropylamino)piperidin-1-yl)pyrimidin-2(1H)-one (D3).

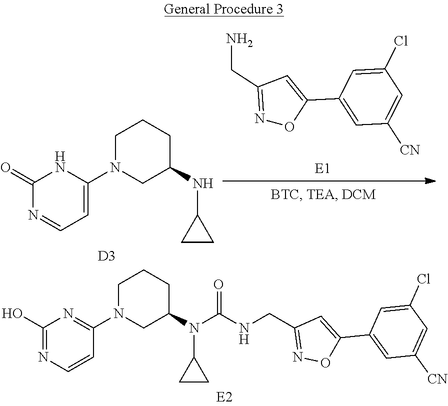

### Synthesis of Compound E2 (Example 6)

To a mixture of compound D3 (70 mg, 0.304 mmol) and TEA (150 mg, 1.52 mmol) in anhydrous DCM (6 mL) at 0° C. under N₂ atmosphere was added compound E1 (84 mg, 0.365 mmol) and a solution of BTC (54 mg, 0.182 mmol) in DCM (5 mL) dropwise. The resulting mixture was stirred at room temperature for 2 hrs and then concentrated under reduced pressure to dryness. The residue was dissolved with EtOAc (20 mL) and washed with water and brine. The organic layer was separated, dried over anhydrous Na₂SO₄ and concentrated to dryness. The crude product was purified by column chromatography on silica gel (eluted with DCM:MeOH=100:0 to 95:5) to afford E2 (15 mg, 10.1 yield) as white solid. LC/MS (ESI) m/z: 494 (M+H)⁺. ¹H NMR (400 MHz, MeOD) δ 8.21-8.12 (m, 2H), 7.90 (d, J=1.5 Hz, 1H), 7.43 (d, J=7.5 Hz, 1H), 6.96 (d, J=3.3 Hz, 1H), 6.18 (d, J=7.3 Hz, 1H), 5.08-4.91 (m, 1H), 4.54-4.42 (m, 2H), 4.18-3.92 (m, 1H), 3.76-3.48 (, 1H), 3.42-3.32 (m, 1H), 3.04-2.80 (m, 1H), 2.60-2.48 (s, 1H), 2.32-2.12 (m, 1H), 2.01-1.79 (1, 2H), 1.62-1.46 (m, 1H), 1.02-0.90 (1, 2H), 0.86-0.70 (, 2H).

| Example | Structure and name | Data |
|---|---|---|
| (1) | 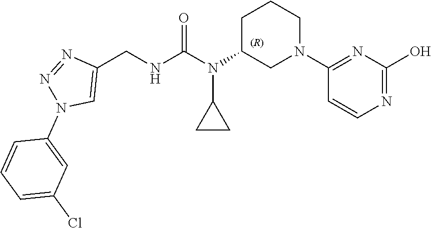 | LC-MS: m/z = 469.2 (M + H) 1H NMR: (400 MHz, MeOD) δ 8.40 (s, 1H), 7.95 (s, 1H), 7.81 (d, J = 8.0 Hz, 1H), 7.56 (t, J = 8.1 Hz, 1H), 7.50 (d, J = 8.1 Hz, 1H), 7.43 (d, J = 7.5 Hz, 1H), 7.00 (s, 1H), 6.18 (d, J = 6.6 Hz, 1H), 4.97 (s, 1H), 4.53 (s, 2H), 4.01 (s, 1H), 3.69 (s, 1H), 3.34 (d, J = 15.0 Hz, 1H), 2.85 (d, J = 104.3 Hz, 1H), 2.55 (s, 1H), 2.24 (dt, J = 11.9, 8.5 Hz, 1H), 1.96 (s, 1H), 1.85 (d, J = 13.1 Hz, 1H), 1.60-1.48 (m, 1H), 0.96 (s, 2H), 0.80 (s, 2H). |
| | 3-{[1-(3-chlorophenyl)-1H-1,2,3- | |
| | triazol-4-yl]methyl}-1-cyclopropyl-1- | |
| | [(3R)-1-(2-hydroxypyrimidin-4- | |
| | yl)piperidin-3-yl]urea | |
| (2) | 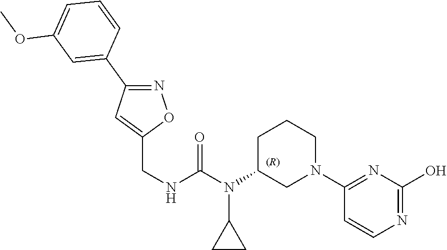 | LC-MS: m/z = 465.2 (M + H) 1H NMR: (400 MHz, MeOD) δ 7.43 (d, J = 7.5 Hz, 1H), 7.39-7.35 (m, 3H), 7.06-7.00 (m, 1H), 6.66 (s, 1H), 6.18 (d, J = 7.2 Hz, 1H), 5.00 (d, J = 18.3 Hz, 1H), 4.54 (s, 2H), 4.12-3.88 (m, 1H), 3.85 (s, 3H), 3.76-3.48 (m, 1H), 3.37 (d, J = 12.0 Hz, 1H), 2.88 (dd, J = 102.4, 20.6 Hz, 1H), 2.58 (s, 1H), 2.26 (td, J = 12.6, 8.5 Hz, 1H), 2.00-1.80 (m, 2H), 1.60- 1.48 (m, 1H), 0.98 (s, 2H), 0.81 (s, 2H). |
| | 1-cyclopropyl-1-[(3R)-1-(2- | |
| | hydroxypyrimidin-4-yl)piperidin-3- | |
| | yl]-3-{[3-(3-methoxyphenyl)-1,2- | |
| | oxazol-5-yl]methyl}urea | |
| (3) | 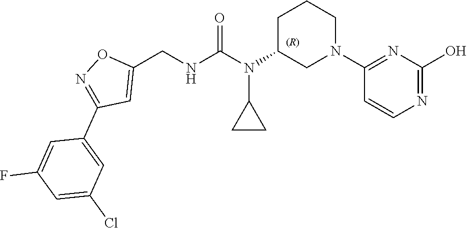 | LC-MS: m/z = 487.1 (M + H) 1H NMR: (400 MHz, MeOD) δ 7.72 (s, 1H), 7.57 (ddd, J = 9.3, 2.3, 1.4 Hz, 1H), 7.43 (d, J = 7.5 Hz, 1H), 7.32 (dt, J = 8.5, 2.1 Hz, 1H), 7.17 (t, J = 5.6 Hz, 1H), 6.74 (s, 1H), 6.18 (d, J = 7.4 Hz, 1H), 4.97 (s, 1H), 4.55 (d, J = 5.0 Hz, 2H), 4.00 (s, 1H), 3.69 (s, 1H), 3.36 (d, J = 12.0 Hz, 1H), 2.94 (d, J = 53.2 Hz, 1H), 2.57 (s, 1H), 2.26 (qd, J = 12.6, 4.2 Hz, 1H), 2.00-1.81 (m, 2H), 1.60-1.47 (m, 1H), 0.98 (s, 2H), 0.82 (s, 2H). |
| | 3-{[3-(3-chloro-5-fluorophenyl)-1,2- | |
| | oxazol-5-yl]methyl}-1-cyclopropyl-1- | |
| | [(3R)-1-(2-hydroxypyrimidin-4- | |
| | yl)piperidin-3-yl]urea | |
| (4) | 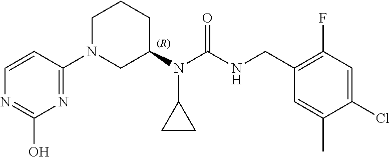 | LC-MS: m/z = 434.2 (M + H) 1H NMR: (400 MHz, MeOD) δ 7.43 (d, J = 7.5 Hz, 1H), 7.25 (d, J = 8.0 Hz, 1H), 7.13 (d, J = 9.8 Hz, 1H), 6.17 (d, J = 7.4 Hz, 1H), 4.97 (s, 1H), 4.37 (s, 2H), 3.99 (d, J = 22.6 Hz, 1H), 3.81-3.53 (m, 1H), 3.35 (d, J = 12.1 Hz, 1H), 2.85 (dd, J = 114.1, 15.0 Hz, 1H), 2.56 (s, 1H), 2.32 (s, 3H), 2.27-2.19 (m, 1H), 1.98-1.81 (m, 2H), 1.53 (q, J = 13.1 Hz, 1H), 0.95 (s, 2H), 0.76 (s, 2H). |
| | 3-[(4-chloro-2-fluoro-5- | |
| | methylphenyl)methyl]-1-cyclopropyl- | |
| | 1-[(3R)-1-(2-hydroxypyrimidin-4- | |
| | yl)piperidin-3-yl]urea | |
| (5) | 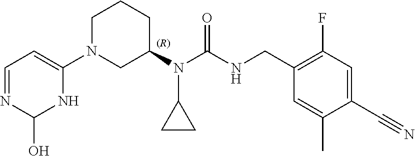 | LC-MS: m/z = 425.3 (M + H) 1H NMR: (400 MHz, MeOD) δ 8.42 (d, J = 8.1 Hz, 1H), 7.52-7.39 (m, 2H), 6.46 (d, J = 8.3 Hz, 1H), 4.70-4.56 (m, 3H), 4.21-3.80 (m, 1H), 3.59-3.41 (m, 1H), 3.28-3.04 (m, 2H), 2.95-2.54 (m, 1H), 2.49 (s, 3H), 2.34- 2.13 (m, 1H), 1.95-1.77 (m, 1H), 1.76-1.53 (m, 2H), 0.83-0.62 (m, 2H), 0.61-0.42 (m, 2H). |
| | 3-[(4-cyano-2-fluoro-5- | |
| | methylphenyl)methyl]-1-cyclopropyl- | |
| | 1-[(3R)-1-(2-oxo-2,3- | |
| | dihydropyrimidin-4-yl)piperidin-3- | |
| | yl]urea | |
| (6) | 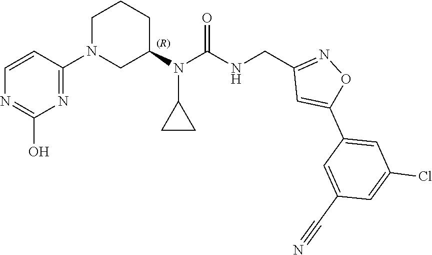 | LC-MS: m/z = 494.1 (M + H) 1H NMR: (400 MHz, MeOD) δ 8.20-8.11 (m, 2H), 7.90 (d, J = 1.5 Hz, 1H), 7.43 (d, J = 7.5 Hz, 1H), 7.06 (dd, J = 12.3, 6.8 Hz, 1H), 6.96 (d, J = 3.3 Hz, 1H), 6.18 (d, J = 7.3 Hz, 1H), 4.91 (s, 1H), 4.48 (d, J = 10.5 Hz, 2H), 4.01 (s, 1H), 3.71 (s, 1H), 3.35 (d, J = 12.3 Hz, 1H), 2.93 (d, J = 53.1 Hz, 1H), 2.57 (s, 1H), 2.26 (qd, J = 12.6, 4.1 Hz, 1H), 2.00- 1.79 (m, 2H), 1.54 (dtd, J = 13.2, 9.2, 4.1 Hz, 1H), 0.97 (s, 2H), 0.86 (d, J = 25.9 Hz, 2H). |
| | 3-{[5-(3-chloro-5-cyanophenyl)-1,2- | |
| | oxazol-3-yl]methyl}-1-cyclopropyl-1- | |
| | [(3R)-1-(2-hydroxypyrimidin-4- | |
| | yl)piperidin-3-yl]urea | |
| (7) | 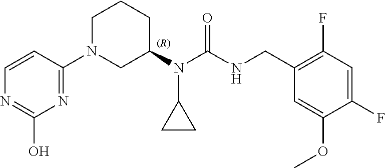 | LC-MS: m/z = 434.2 (M + H) 1H NMR: (400 MHz, MeOD) δ 7.43 (d, J = 7.5 Hz, 1H), 7.14-7.02 (m, 1H), 6.97-6.89 (m, 1H), 6.17 (d, J = 7.3 Hz, 1H), 4.91 (d, J = 39.8 Hz, 1H), 4.38 (s, 2H), 4.03 (s, 1H), 3.85 (s, 3H), 3.67 (s, 1H), 3.50-3.34 (m, 1H), 3.09-2.92 (m, 1H), 2.56 (s, 1H), 2.30-2.19 (m, 1H), 1.98-1.80 (m, 2H), 1.55 (t, J = 13.2 Hz, 1H), 0.95 (s, 1H), 0.76 (s, 2H). |
| | 1-cyclopropyl-3-[(2,4-difluoro-5- | |
| | methoxyphenyl)methyl]-1-[(3R)-1-(2- | |
| | hydroxypyrimidin-4-yl)piperidin-3- | |
| | yl]uea | |
| (8) | 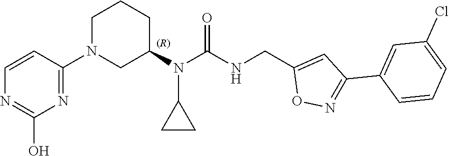 | LC-MS: m/z = 469.1 (M + H) 1H NMR: (400 MHz, MeOD) δ 7.86 (s, 1H), 7.76 (dd, J = 4.8, 2.4 Hz, 1H), 7.52-7.40 (m, 3H), 6.70 (s, 1H), 6.19 (d, J = 7.2 Hz, 1H), 4.87 (s, 1H), 4.55 (s, 2H), 4.01 (s, 1H), 3.63 (s, 1H), 3.37 (d, J = 12.2 Hz, 1H), 3.01 (s, 1H), 2.58 (s, 1H), 2.35-2.17 (m, 1H), 1.93 (dd, J = 57.5, 18.5 Hz, 2H), 1.54 (q, J = 13.4 Hz, 1H), 0.90 (d, J = 66.9 Hz, 4H). |
| | 3-{[3-(3-chlorophenyl)-1,2-oxazol-5- | |
| | yl]methyl}-1-cyclopropyl-1-[(3R)-1- | |
| | (2-hydroxypyrimidin-4-yl)piperidin-3- | |
| | yl]urea | |
| (9) | 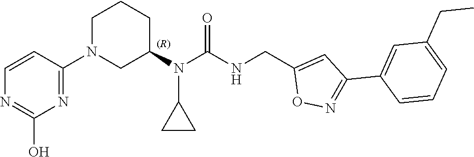 | LC-MS: m/z = 463.2 (M + H) 1H NMR: (400 MHz, MeOD) δ 7.66 (s, 1H), 7.61 (d, J = 7.5 Hz, 1H), 7.45-7.35 (m, 2H), 7.31 (d, J = 7.6 Hz, 1H), 7.16 (s, 1H), 6.66 (s, 1H), 6.18 (d, J = 7.2 Hz, 1H), 4.96 (s, 1H), 4.54 (s, 2H), 4.00 (s, 1H), 3.64 (s, 1H), 3.04 (d, J = 7.2 Hz, 1H), 2.70 (t, J = 7.6 Hz, 2H), 2.58 (s, 1H), 2.26 (dd, J = 21.2, 12.6 Hz, 1H), 1.97 (s, 1H), 1.85 (d, J = 13.9 Hz, 1H), 1.54 (d, J = 13.2 Hz, 1H), 1.27 (dd, J = 14.1, 6.5 |
| | 1-cyclopropyl-3-{[3-(3-ethylphenyl)- | Hz, 4H), 0.98 (s, 2H), 0.82 (s, 2H). |
| | 1,2-oxazol-5-yl]methyl}-1-[(3R)-1-(2- | |
| | hydroxypyrimidin-4-yl)piperidin-3- | |
| | yl]urea | |
| (10) | 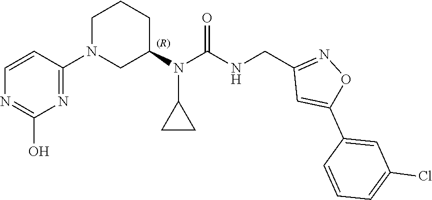 | LC-MS: m/z = 469.2 (M + H) 1H NMR: (400 MHz, MeOD) δ 7.90-7.69 (m, 2H), 7.54-7.36 (m, 3H), 6.80 (s, 1H), 6.19 (d, J = 6.8 Hz, 1H), 4.48 (s, 2H), 4.03 (dt, J = 31.7, 16.0 Hz, 1H), 3.79-3.34 (m, 2H), 3.26-2.98 (m, 1H), 2.98-2.44 (m, 2H), 2.33-2.20 (m, 1H), 2.02-1.81 (m, 2H), 1.55 (dt, J = 19.9, 10.0 Hz, 1H), 0.90 (ddd, J = 44.2, 13.1, 7.2 Hz, 4H). |
| | 3-{[5-(3-chlorophenyl)-1,2-oxazol-3- | |
| | yl]methyl}-1-cyclopropyl-1-[(3R)-1- | |
| | (2-hydroxypyrimidin-4-yl)piperidin-3- | |
| | yl]urea | |
| (11) | 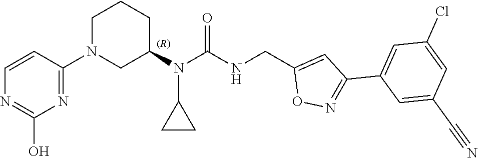 | LC-MS: m/z = 494.1 (M + H) 1H NMR: (400 MHz, MeOD) δ 8.18 (dd, J = 12.4, 6.2 Hz, 2H), 7.91 (s, 1H), 7.43 (d, J = 7.5 Hz, 1H), 7.18 (s, 1H), 6.81 (s, 1H), 6.18 (d, J = 7.5 Hz, 1H), 4.96 (s, 1H), 4.56 (d, J = 4.9 Hz, 2H), 3.86 (d, J = 126.0 Hz, 2H), 3.35 (d, J = 12.3 Hz, 1H), 3.00 (s, 1H), 2.58 (s, 1H), 2.25 (tt, J = 12.6, 6.3 Hz, 1H), 1.97 (s, 1H), 1.85 (d, J = 13.1 Hz, 1H), 1.60-1.47 (m, 1H), 0.98 (s, 2H), 0.83 (s, 2H). |
| | 3-{[3-(3-chloro-5-cyanophenyl)-1,2- | |
| | oxazol-5-yl]methyl}-1-cyclopropyl-1- | |
| | [(3R)-1-(2-hydroxypyrimidin-4- | |
| | yl)piperidin-3-yl]urea | |

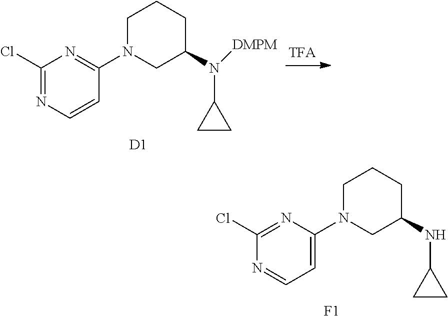

### Step 1: Synthesis of Common Intermediate F1

Compound D1 (3.8 g, 9.45 mmol) was added into TFA (60 mL) in portions at 0° C. under N₂ atmosphere. Then the resulting mixture was stirred at 80° C. for 5 hrs. After cooling to room temperature, the mixture was concentrated under reduced pressure to give crude compound F1 (2.3 g, 96.6% yield) as purple oil which was used at the next step directly without further purification. LC/MS (ESI) m/z: 253 (M+H)⁺.

### Examples 12-19

The compounds in the table below were prepared from the appropriate starting materials, prepared as described above or commercially available, using an appropriate modification of General Procedure 4 and (R)-1-(2-chloropyrimidin-4-yl)-N-cyclopropylpiperidin-3-amine (F1).

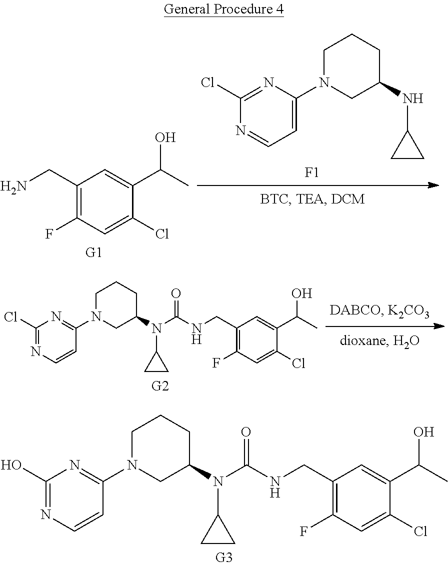

### Step 1: Synthesis of Compound G2

To a mixture of compound G1 (53 mg, 0.264 mmol) and compound F1 (67 mg, 0.264 mmol) in anhydrous DCM (5 mL) were added TEA (80 mg, 0.792 mmol) and a solution of triphosgene (55 mg, 0.185 mmol) in DCM (2 mL) dropwise at −50° C. The resulting mixture was stirred at 45° C. for 1 hr. Then the mixture was diluted with H₂O (20 mL) and extracted with DCM (20 mL×2). The combined organic layers were washed with brine, dried over anhydrous Na₂SO₄, filtered and concentrated to dryness. The residue was purified by column chromatography on silica gel (eluted with DCM:MeOH=100:1 to 100:5) to give compound G2 (90 mg, 71.67% yield) as white solid. LC/MS (ESI) m/z: 482 (M+H)⁺.

### Step 2: Synthesis of Compound G3 (Example 15)

To a solution of compound G2 (90 mg, 0.187 mmol) in dioxane (3 mL) and H₂O (3 mL) were added DABCO (84 mg, 0.748 mmol) and K₂CO₃ (155 mg, 1.122 mmol), the resulting mixture was stirred at 80° C. for 40 hrs. Then the mixture was diluted with H₂O (25 mL) and extracted with EtOAc (25 mL×2). The combined organic layers were washed with brine, dried over anhydrous Na₂SO₄, filtered and concentrated to dryness. The residue was purified by prep-HPLC (Gemini 5 μm C18 250*21.2 mm, H₂O/MeCN (5-95%)/0.1% HCOOH) to give compound G3 (12.2 mg, 14.08% yield) as light-yellow solid. LC/MS (ESI) m/z: 464 (M+H)⁺.

| Example | Structure and name | Data |
|---|---|---|
| (12) | 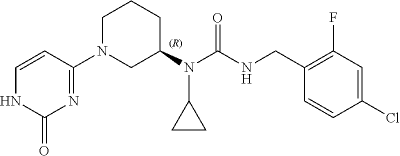 | LC-MS: m/z = 420.1 (M + H) 1H NMR: (400 MHz, DMSO) δ 10.46 (s, 1H), 7.41 (d, J = 7.4 Hz, 1H), 7.39 − 7.31 (m, 2H), 7.28 (d, J = 8.3 Hz, 1H), 6.95 (t, J = 5.5 Hz, 1H), 5.99 (d, J = 7.3 Hz, 1H), 4.59 (d, J = 93.0 Hz, 1H), 4.30 (d, J = 5.4 Hz, 2H), 4.20 − 3.65 (m, 1H), 3.59 − 3.42 (m, 1H), 3.14 (s, 1H), 2.67 (s, 1H), 2.47 (s, 1H), 2.18 − 2.02 (m, 1H), 1.89 − 1.65 (m, 2H), 1.36 (q, J = 12.6 Hz, 1H), 0.89 (d, J = 6.0 Hz, 2H), 0.69 (s, 2H). |
| | 1-[(4-chloro-2-fluorophenyl)methyl]- | |
| | 3-cyclopropyl-3-[(3R)-1-(2-oxo-1,2- | |
| | dihydropyrimidin-4-yl)piperidin-3- | |
| | yl]urea | |
| (13) | 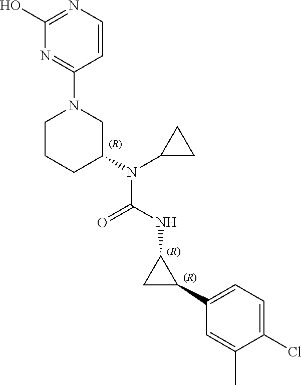 | LC-MS: m/z = 442.2 (M + H) 1H NMR: 1H NMR (400 MHz, MeOD) δ 7.43 (d, J = 7.5 Hz, 1H), 7.20 (d, J = 8.2 Hz, 1H), 7.08 (s, 1H), 6.94 (dd, J = 8.2, 1.7 Hz, 1H), 6.66 (s, 1H), 6.18 (d, J = 5.2 Hz, 1H), 4.97 (s, 1H), 4.00 (s, 1H), 3.67 (s, 1H), 3.39 − 3.32 (m, 1H), 3.11 − 2.64 (m, 2H), 2.49 (s, 1H), 2.32 (s, 3H), 2.30 − 2.19 (m, 1H), 2.04 − 1.89 (m, 2H), 1.85 (d, J = 13.9 Hz, 1H), 1.61 − 1.46 (m, 1H), 1.26 − 1.13 (m, 2H), 0.92 (s, 2H), 0.73 (s, 2H). |
| | 3-[(1S,2R)-2-(4-chloro-3- | |
| | methylphenyl)cyclopropyl]-1- | |
| | cyclopropyl-1-[(3R)-1-(2- | |
| | hydroxypyrimidin-4-yl)piperidin-3- | |
| | yl]urea | |
| (14) | 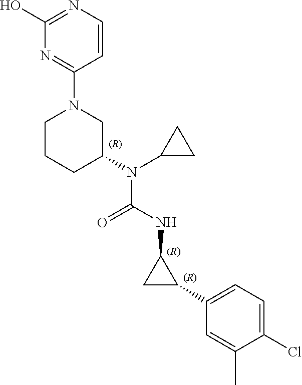 | LC-MS: m/z = 442.2 (M + H) 1H NMR: (400 MHz, MeOD) δ 7.43 (d, J = 7.5 Hz, 1H), 7.20 (d, J = 8.2 Hz, 1H), 7.08 (s, 1H), 6.94 (d, J = 6.7 Hz, 1H), 6.66 (s, 1H), 6.18 (d, J = 6.8 Hz, 1H), 4.96 (s, 1H), 4.00 (s, 1H), 3.68 (s, 1H), 3.38 − 3.32 (m, 1H), 3.10 − 2.63 (m, 2H), 2.49 (s, 1H), 2.30 (d, J = 10.6 Hz, 3H), 2.28 − 2.16 (m, 1H), 2.06 − 1.90 (m, 2H), 1.84 (d, J = 13.0 Hz, 1H), 1.61 − 1.46 (m, 1H), 1.26 − 1.13 (m, 2H), 0.93 (s, 2H), 0.73 (s, 2H). |
| | 3-[(1R,2S)-2-(4-chloro-3- | |
| | methylphenyl)cyclopropyl]-1- | |
| | cyclopropyl-1-[(3R)-1-(2- | |
| | hydroxypyrimidin-4-yl)piperidin-3- | |
| | yl]urea | |
| (15) | 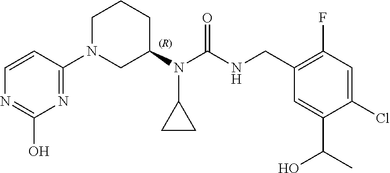 3-{[4-chloro-2-fluoro-5-(1- | LC-MS: m/z = 464.2 (M + H) 1H NMR: (400 MHz, MeOD) δ 7.62 (d, J = 8.3 Hz, 1H), 7.43 (d, J = 7.4 Hz, 1H), 7.12 (d, J = 9.7 Hz, 1H), 6.97 (s, 1H), 6.18 (d, J = 7.4 Hz, 1H), 5.16 (qd, J = 6.3, 2.7 Hz, 1H), 4.40 (d, J = 20.6 Hz, 2H), 3.99 (s, 1H), 3.57 (d, J = 71.4 Hz, 1H), 3.35 (s, 1H), 3.16 (d, J = 25.0 Hz, 1H), 2.79 (d, J = 108.2 Hz, 1H), 2.58 (s, 1H), 2.25 (qd, J = 12.5, 4.2 Hz, 1H), 2.03 − 1.90 (m, 1H), 1.84 (d, J = 13.0 Hz, 1H), 1.58 − 1.46 (m, 1H), 1.38 (dd, J = 6.4, 1.0 Hz, 3H), 0.89 (d, J = 65.9 Hz, 4H). |
| | hydroxyethyl)phenyl]methyl}-1- | |
| | cyclopropyl-1-[(3R)-1-(2- | |
| | hydroxypyrimidin-4-yl)piperidin-3- | |
| | yl]urea | |
| (16) | 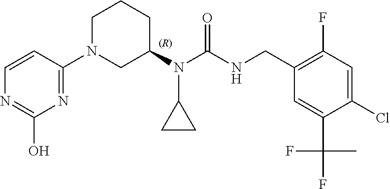 | LC-MS: m/z = 484.2 (M + H) 1H NMR: (400 MHz, MeOD) δ 7.63 (d, J = 7.8 Hz, 1H), 7.44 (d, J = 7.5 Hz, 1H), 7.31 (d, J = 9.6 Hz, 1H), 6.18 (d, J = 7.5 Hz, 1H), 4.43 (s, 2H), 4.01 (s, 1H), 3.56 (d, J = 55.6 Hz, 1H), 3.37 (t, J = 12.1 Hz, 1H), 3.08 (d, J = 40.1 Hz, 1H), 2.87 (dd, J = 66.1, 41.5 Hz, 1H), 2.57 (s, 1H), 2.25 (tt, J = 12.3, 6.1 Hz, 1H), 2.05 − 1.96 (m, 3H), 1.85 (d, J = 12.8 Hz, 2H), 1.58 − 1.49 (m, 1H), 0.96 (s, 2H), 0.77 (s, 2H). |
| | 3-{[4-chloro-5-(1,1-difluoroethyl)-2- | |
| | fluorophenyl]methyl}-1-cyclopropyl- | |
| | 1-[(3R)-1-(2-hydroxypyrimidin-4- | |
| | yl)piperidin-3-yl]urea | |
| (17) | 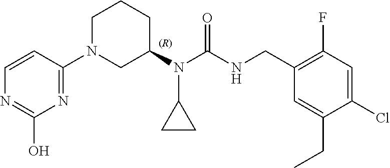 | LC-MS: m/z = 448.0 (M + H) 1H NMR: (400 MHz, MeOD) δ 8.48 (s, 1H), 7.43 (d, J = 7.5 Hz, 1H), 7.25 (d, J = 8.0 Hz, 1H), 7.12 (d, J = 9.8 Hz, 1H), 6.91 (s, 1H), 6.17 (d, J = 7.5 Hz, 1H), 4.39 (s, 2H), 4.00 (s, 1H), 3.56 (d, J = 72.2 Hz, 1H), 3.35 (d, J = 11.6 Hz, 2H), 3.07 − 2.67 (m, 3H), 2.56 (s, 1H), 2.25 (dt, J = 12.4, 8.2 Hz, 1H), 1.98 − 1.80 (m, 2H), 1.59 − 1.47 (m, 1H), 1.20 (t, J = 7.5 Hz, 3H), 1.00 − 0.90 (m, 2H), 0.87 − 0.66 (m, 2H) |
| | 3-[(4-chloro-5-ethyl-2- | |
| | fluorophenyl)methyl]-1-cyclopropyl- | |
| | 1-[(3R)-1-(2-hydroxypyrimidin-4- | |
| | yl)piperidin-3-yl]urea | |
| (18) | 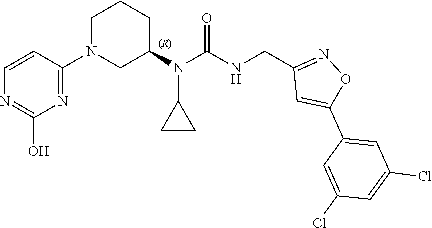 | LC-MS: m/z= 503.1 (M + H) 1H NMR: (400 MHz, MeOD) δ 7.81 (d, J = 1.8 Hz, 2H), 7.56 (t, J = 1.8 Hz, 1H), 7.43 (d, J = 7.5 Hz, 1H), 7.06 (s, 1H), 6.88 (s, 1H), 6.19 (d, J = 7.2 Hz, 1H), 4.99 (s, 1H), 4.49 (d, J = 4.7 Hz, 2H), 4.15 − 3.51 (m, 2H), 3.36 (d, J = 12.2 Hz, 1H), 3.08 − 2.67 (m, 1H), 2.56 (s, 1H), 2.33 − 2.20 (m, 1H), 1.98 (s, 1H), 1.89 − 1.81 (m, 1H), 1.61 − 1.47 (m, 1H), 0.96 (s, 2H), 0.83 (s, 2H). |
| | 1-cyclopropyl-3-{[5-(3,5- | |
| | dichlorophenyl)-1,2-oxazol-3- | |
| | yl]methyl}-1-[(3R)-1-(2- | |
| | hydroxypyrimidin-4-yl)piperidin-3- | |
| | yl]urea | |
| (19) | 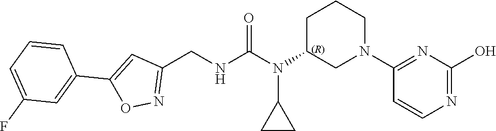 1-cyclopropyl-3-{[5-(3-fluorophenyl)- | LC-MS: m/z = 453.2 (M + H) 1H NMR: (400 MHz, MeOD) δ 7.65 (d, J = 7.8 Hz, 1H), 7.61 − 7.48 (m, 2H), 7.43 (d, J = 7.5 Hz, 1H), 7.21 (td, J = 8.7, 2.2 Hz, 1H), 6.79 (s, 1H), 6.19 (d, J = 7.0 Hz, 1H), 4.48 (s, 2H), 4.02 (t, J = 26.1 Hz, 1H), 3.85 − 3.50 (m, 1H), 3.37 (d, J = 11.8 Hz, 1H), 3.30 − 2.62 (m, 2H), 2.57 (s, 1H), 2.26 (qd, J = 12.7, 4.2 Hz, 1H), 2.03 − 1.80 (m, 2H), 1.55 (ddd, J = 22.3, 13.0, 8.6 Hz, 1H), 1.04 − 0.68 (m, 4H). |
| | 1,2-oxazol-3-yl]methyl}-1-[(3R)-1-(2- | |
| | hydroxypyrimidin-4-yl)piperidin-3- | |
| | yl]urea | |

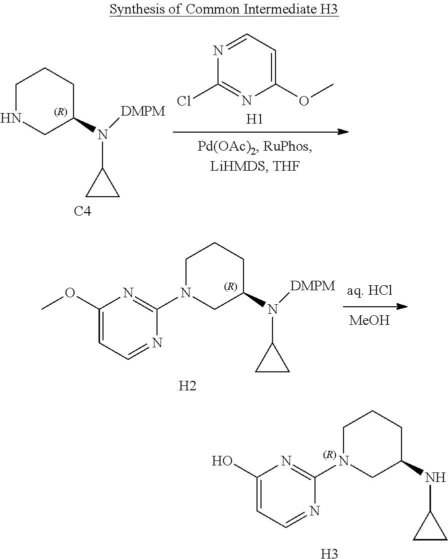

### Step 1: Synthesis of Compound H2

To a mixture of compound H1 (800 mg, 5.534 mmol) and compound C₄ (1.6 g, 5.534 mmol) in THE (40 mL) was added Pd(OAc)₂ (124 mg, 0.533 mmol) and RuPhos (258 mg, 0.553 mmol) followed by addition of Lithium bis(trimethylsilyl)amide (25 mL, 1 M in THF) in portions at 0° C. under N₂ atmosphere. The resulting mixture was stirred at 75° C. for 16 hrs. Then the mixture was cooled and concentrated under reduced pressure to dryness. The residue was diluted with DCM (100 mL) and washed with saturated NH₄Cl solution and brine. The organic layer was separated, dried over anhydrous Na₂SO₄ and concentrated to dryness. The crude product was purified by column chromatography on silica gel (eluted with DCM:MeOH=100:0 to 96:4) to afford compound H2 (900 mg, 40.8% yield) as a light-yellow oil. LC/MS (ESI) m/z: 399 (M+H)⁺. ¹H NMR (400 MHz, MeOD) δ 8.01 (d, J=5.7 Hz, 1H), 6.03 (d, J=5.7 Hz, 1H), 4.68-4.58 (m, 1H), 4.32-4.22 (m, 1H), 3.89 (s, 3H), 3.28-3.19 (m, 2H), 3.04-2.95 (m, 1H), 2.53-2.46 (m, 1H), 2.17-2.07 (m, 1H), 1.84-1.75 (m, 1H), 1.63-1.54 (m, 2H), 0.73-0.64 (m, 2H), 0.63-0.55 (m, 2H).

### Step 2: Synthesis of H3

To a solution of H2 (100 mg, 0.251 mmol) in MeOH (10 mL) was added conc. HCl (10 mL, 12 N). The resulting mixture was stirred at 90° C. for 48 hrs under N₂ atmosphere. Then the mixture was cooled, concentrated under reduced pressure to dryness. The residue was diluted with water (30 mL) and basified with saturated NaHCO₃ solution to pH=8. Then the mixture was extracted with EtOAc (30 mL×2). The combined organic layers was washed with water and brine, dried over Na₂SO₄, concentrated to dryness. The crude product was purified by column chromatography on silica gel (eluted with DCM:MeOH=100:0 to 95:5) to afford H3 (40 mg, 64.1% yield) as light-brown oil. LC/MS (ESI) m/z: 235 (M+H)⁺. ¹H NMR (400 MHz, MeOD) δ 7.61 (d, J=6.7 Hz, 1H), 5.74 (d, J=6.7 Hz, 1H), 4.37-4.31 (m, 1H), 4.01-3.91 (m, 1H), 3.19-3.12 (m, 1H), 3.08-3.01 (m, 1H), 2.88-2.76 (m, 1H), 2.38-2.26 (m, 1H), 2.12-2.03 (m, 1H), 1.88-1.76 (m, 1H), 1.64-1.55 (m, 1H), 1.53-1.41 (m, 1H), 0.60-0.50 (m, 2H), 0.44-0.32 (m, 2H).

### Examples 20-28

The compounds in the table below were prepared from the appropriate starting materials, prepared as described above or commercially available, using an appropriate modification of General Procedure 5 and (R)-2-(3-(cyclopropylamino)piperidin-1-yl)pyrimidin-4-ol.

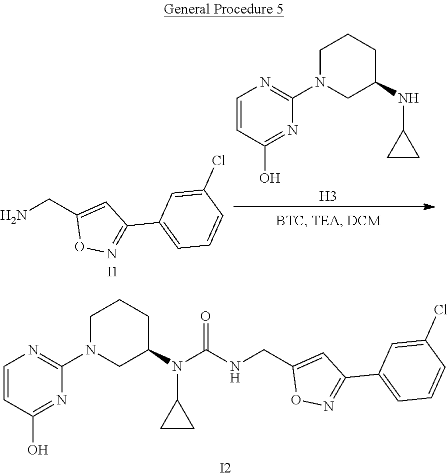

### Step 1: Synthesis of Compound 12 (Example 20)

To a mixture of compound I1 (80 mg, 0.384 mmol), TEA (190 mg, 1.92 mmol) and compound H3 (108 mg, 0.462 mmol) in anhydrous DCM (8 mL) at −50° C. under N₂ atmosphere was added a solution of BTC (68 mg, 0.230 mmol) in DCM (5 mL) dropwise. The resulting mixture was stirred at room temperature for 2 hrs and then concentrated under reduced pressure to dryness. The residue was dissolved with EtOAc (30 mL) and washed with water and brine. The organic layer was separated, dried over anhydrous Na₂SO₄ and concentrated to dryness. The crude product was purified by column chromatography on silica gel (eluted with DCM:MeOH=100:0 to 95:5) to afford 12 (20 mg, 11.1% yield) as white solid.

| Example | Structure and name | Data |
|---|---|---|
| (20) | 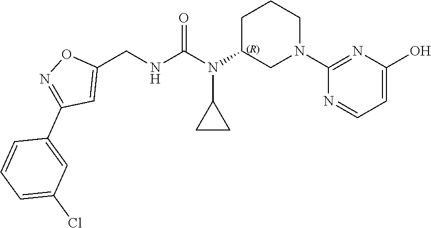 | LC-MS: m/z = 469.2 (M + H) 1H NMR: (400 MHz, DMSO) δ 11.13 (s, 1H), 7.94 (s, 1H), 7.86 (dd, J = 6.9, 1.7 Hz, 1H), 7.69 (s, 1H), 7.60 − 7.50 (m, 2H), 7.08 (t, J = 5.7 Hz, 1H), 6.89 (s, 1H), 5.61 (s, 1H), 4.41 (dd, J = 27.0, 8.4 Hz, 4H), 3.78 − 3.40 (m, 2H), 3.18 (t, J = 11.9 Hz, 1H), 2.76 − 2.67 (m, 1H), 2.06 (tt, J = 12.5, 6.3 Hz, 1H), 1.76 (dd, J = 35.4, 12.4 Hz, 2H), 1.44 (t, J = 13.0 Hz, 1H), 0.89 (t, J = 7.8 Hz, 2H), 0.80 − 0.69 (m, 2H). |
| | 3-{[3-(3-chlorophenyl)-1,2-oxazol-5- | |
| | yl]methyl}-1-cyclopropyl-1-[(3R)-1- | |
| | (4-hydroxypyrimidin-2-yl)piperidin-3- | |
| | yl]urea | |
| (21) | 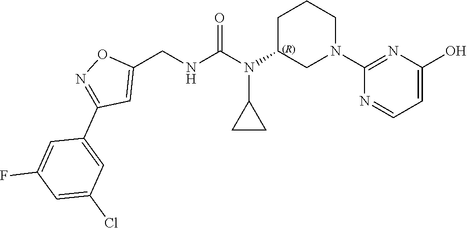 | LC-MS: m/z = 487.2 (M + H) 1H NMR: (400 MHz, MeOD) δ 7.72 (s, 1H), 7.59 (t, J = 9.4 Hz, 2H), 7.39 − 7.26 (m, 1H), 6.74 (s, 1H), 5.75 (d, J = 6.7 Hz, 1H), 4.56 (s, 2H), 4.36 − 4.22 (m, 2H), 3.76 − 3.67 (m, 1H), 3.36 (d, J = 12.0 Hz, 1H), 2.87 (t, J = 11.9 Hz, 1H), 2.63 − 2.54 (m, 1H), 2.29 − 2.17 (m, 1H), 1.97 (d, J = 10.5 Hz, 1H), 1.86 (d, J = 13.4 Hz, 1H), 1.67 − 1.53 (m, 1H), 0.98 (dd, J = 6.4, 3.3 Hz, 2H), 0.83 (d, J = 4.2 Hz, 2H). |
| | 3-{[3-(3-chloro-5-fluorophenyl)-1,2- | |
| | oxazol-5-yl]methyl}-1-cyclopropyl-1- | |
| | [(3R)-1-(4-hydroxypyrimidin-2- | |
| | yl)piperidin-3-yl]urea | |
| (22) | 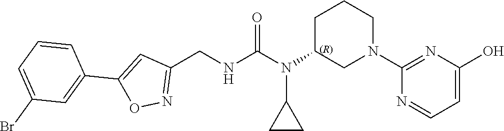 3-{[5-(3-bromophenyl)-1,2-oxazol-3- yl]methyl}-1-cyclopropyl-1-[(3R)-1- (4-hydroxypyrimidin-2-yl)piperidin-3-yl]urea | LC-MS: m/z = 514.0 (M + H) 1H NMR: 1H NMR (400 MHz, MeOD) δ 7.99 (d, J = 1.6 Hz, 1H), 7.80 (d, J = 7.9 Hz, 1H), 7.62 (t, J = 7.5 Hz, 2H), 7.42 (t, J = 8.0 Hz, 1H), 6.80 (s, 1H), 5.74 (d, J = 6.7 Hz, 1H), 4.51 (d, J = 18.7 Hz, 2H), 4.29 (dd, J = 20.8, 14.6 Hz, 2H), 3.78 − 3.69 (m, 1H), 3.36 (d, J = 12.1 Hz, 1H), 2.87 (dd, J = 13.2, 10.8 Hz, 1H), 2.60 − 2.53 (m, 1H), 2.29 − 2.17 (m, 1H), 1.97 (d, J = 11.1 Hz, 1H), 1.86 (d, J = 13.4 Hz, 1H), 1.67 − 1.54 (m, 1H), 0.96 (dt, J = 13.5, 6.8 Hz, 2H), 0.84 (d, J = 3.4 Hz, 2H). |
| (23) | 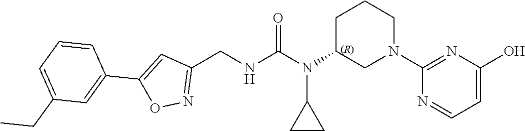 1-cyclopropyl-3-{[5-(3-ethylphenyl)- 1,2-oxazol-3-yl]methyl}-1-[(3R)-1-(4- hydroxypyrimidin-2-yl)piperidin-3-yl]urea | LC-MS: m/z = 463.3 (M + H) 1H NMR: (400 MHz, DMSO) δ 11.11 (s, 1H), 7.72 − 7.53 (m, 3H), 7.44 (t, J = 7.7 Hz, 1H), 7.35 (d, J = 7.7 Hz, 1H), 6.97 (t, J = 5.7 Hz, 1H), 6.84 (s, 1H), 5.58 (s, 1H), 4.36 (d, J = 5.8 Hz, 4H), 3.57 (s, 1H), 3.19 (s, 1H), 2.68 (dd, J = 15.1, 7.5 Hz, 3H), 2.47 (d, J = 3.6 Hz, 1H), 2.07 (dt, J = 12.4, 8.9 Hz, 1H), 1.76 (dd, J = 33.3, 12.7 Hz, 2H), 1.43 (d, J = 13.3 Hz, 1H), 1.21 (d, J = 7.6 Hz, 3H), 0.88 (d, J = 6.4 Hz, 2H), 0.76 − 0.67 (m, 2H). |
| (24) | 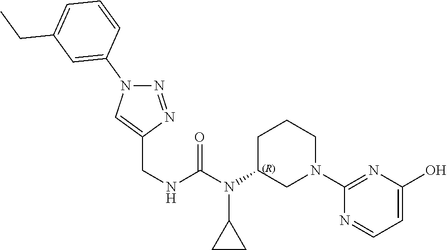 | LC-MS: m/z = 463.2 (M + H) 1H NMR: (400 MHz, DMSO) δ 11.11 (s, 1H), 8.52 (s, 1H), 7.91 − 7.58 (m, 3H), 7.49 (t, J = 7.8 Hz, 1H), 7.33 (d, J = 7.8 Hz, 1H), 6.87 (t, J = 5.6 Hz, 1H), 5.60 (s, 1H), 4.41 (d, J = 5.6 Hz, 4H), 3.56 (d, J = 11.8 Hz, 1H), 3.16 (d, J = 12.0 Hz, 1H), 2.71 (dt, J = 11.5, 5.6 Hz, 3H), 2.45 (s, 1H), 2.06 (dt, J = 13.4, 10.2 Hz, 1H), 1.75 (dd, J = 31.6, 11.8 Hz, 2H), 1.42 (d, J = 12.8 Hz, 1H), 1.24 (t, J = 7.0 Hz, 3H), 0.86 (t, J = 8.1 Hz, 2H), 0.76 − 0.63 (m, 2H). |
| | 1-cyclopropyl-3-{[1-(3-ethylphenyl)- | |
| | 1H-1,2,3-triazol-4-yl]methyl}-1- | |
| | [(3R)-1-(4-hydroxypyrimidin-2- | |
| | yl)piperidin-3-yl]urea | |
| (25) | 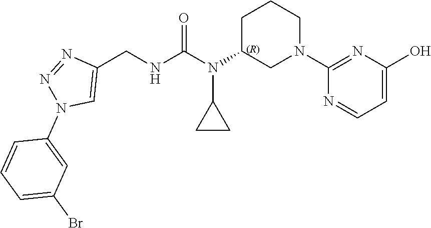 | LC-MS: m/z = 513.2 (M + H) 1H NMR: (400 MHz, MeOD) δ 8.41 (s, 1H), 8.09 (s, 1H), 7.86 (d, J = 8.1 Hz, 1H), 7.76 − 7.38 (m, 3H), 7.04 (s, 1H), 5.73 (d, J = 6.1 Hz, 1H), 4.53 (s, 2H), 4.28 (dd, J = 24.9, 13.0 Hz, 2H), 3.74 (t, J = 11.8 Hz, 1H), 2.87 (t, J = 12.0 Hz, 1H), 2.58 − 2.50 (m, 1H), 2.20 (dd, J = 12.5, 3.9 Hz, 1H), 1.96 (d, J = 11.6 Hz, 1H), 1.85 (d, J = 13.3 Hz, 1H), 1.59 (dd, J = 9.0, 4.0 Hz, 1H), 1.30 (d, J = 10.3 Hz, 1H), 0.95 (dd, J = 6.3, 3.1 Hz, 2H), 0.81 (d, J = 4.0 Hz, 2H). |
| | 3-{[1-(3-bromophenyl)-1H-1,2,3- | |
| | triazol-4-yl]methyl}-1-cyclopropyl-1- | |
| | [(3R)-1-(4-hydroxypyrimidin-2- | |
| | yl)piperidin-3-yl]urea | |
| (26) | 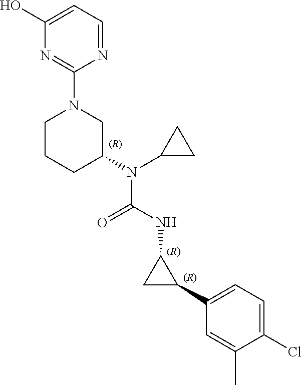 | LC-MS: m/z = 442.2 (M + H) 1H NMR: (400 MHz, MeOD) δ 7.60 (s, 1H), 7.20 (d, J = 8.2 Hz, 1H), 7.09 (s, 1H), 6.95 (d, J = 8.0 Hz, 1H), 5.74 (d, J = 6.8 Hz, 1H), 4.28 (s, 2H), 3.69 (s, 1H), 3.36 (s, 1H), 2.86 (t, J = 12.1 Hz, 1H), 2.72 (d, J = 3.1 Hz, 1H), 2.49 (s, 1H), 2.32 (s, 3H), 2.25 − 2.15 (m, 1H), 1.97 (d, J = 18.2 Hz, 2H), 1.85 (d, J = 14.9 Hz, 1H), 1.58 (d, J = 14.1 Hz, 1H), 1.23 − 1.15 (m, 2H), 0.93 (s, 2H), 0.75 (s, 2H). |
| | 3-[(1S,2R)-2-(4-chloro-3- | |
| | methylphenyl)cyclopropyl]-1- | |
| | cyclopropyl-1-[(3R)-1-(4- | |
| | hydroxypyrimidin-2-yl)piperidin-3- | |
| | yl]urea | |
| (27) | 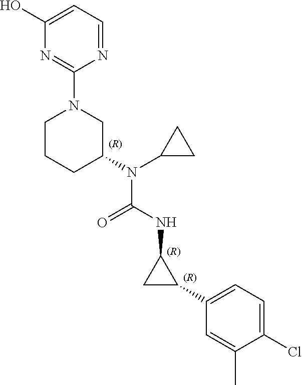 | LC-MS: m/z = 442.2 (M + H) 1H NMR: (400 MHz, DMSO) δ 7.59 (s, 1H), 7.20 (d, J = 8.3 Hz, 1H), 7.08 (s, 1H), 6.94 (d, J = 8.3 Hz, 1H), 5.73 (d, J = 7.0 Hz, 1H), 4.26 (dd, J = 29.5, 13.3 Hz, 2H), 3.69 (s, 1H), 3.31 (s, 1H), 2.84 (t, J = 12.4 Hz, 1H), 2.71 (s, 1H), 2.47 (s, 1H), 2.31 (s, 3H), 2.18 (t, J = 11.4 Hz, 1H), 2.00 − 1.90 (m, 2H), 1.84 (d, J = 13.3 Hz, 1H), 1.57 (d, J = 13.4 Hz, 1H), 1.23 − 1.13 (m, 2H), 0.91 (s, 2H), 0.73 (s, 2H). |
| | 3-[(1R,2S)-2-(4-chloro-3- | |
| | methylphenyl)cyclopropyl]-1- | |
| | cyclopropyl-1-[(3R)-1-(4- | |
| | hydroxypyrimidin-2-yl)piperidin-3- | |
| | yl]urea | |
| (28) | 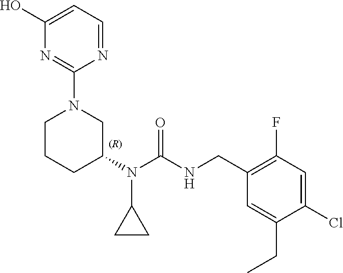 | LC-MS: m/z = 448.2 (M + H) 1H NMR: 400 MHz, MeOD) δ 7.86 − 7.78 (m, 1H), 7.26 (d, J = 8.1 Hz, 1H), 7.15 (d, J = 9.7 Hz, 1H), 6.15 (dd, J = 6.8, 4.3 Hz, 1H), 4.41 (q, J = 15.6 Hz, 2H), 4.33 − 4.20 (m, 2H), 3.79 (t, J = 11.8 Hz, 1H), 3.46 (t, J = 12.1 Hz, 1H), 3.06 (t, J = 13.2 Hz, 1H), 2.80 − 2.68 (m, 2H), 2.56 (s, 1H), 2.33 − 2.20 (m, 1H), 2.05 − 1.91 (m, 2H), 1.74 − 1.59 (m, 1H), 1.21 (t, J = 7.3 Hz, 3H), 0.99 (d, J = 6.4 Hz, 2H), 0.84 − 0.68 (m, 2H). |
| | 1-[(4-chloro-5-ethyl-2- | |
| | fluorophenyl)methyl]-3-cyclopropyl- | |
| | 3-[(3R)-1-(4-hydroxypyrimidin-2- | |
| | yl)piperidin-3-yl]urea | |

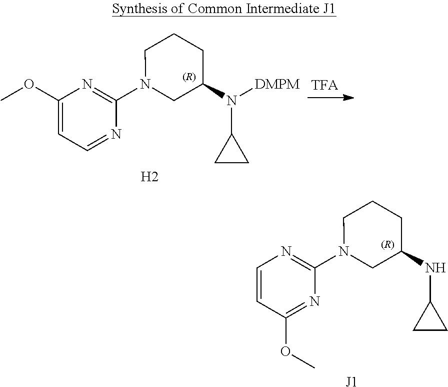

### Step 1: Synthesis of Compound E3

Compound H2 (400 mg, 1.004 mmol) was added to TFA (8 mL) and the resulting mixture was stirred at 80° C. for 3 hrs under N₂ atmosphere. After cooling, the mixture was concentrated under reduced pressure to give crude J1 (230 mg, 92.27% yield) as purple solid without further purification. LC/MS (ESI) m/z: 249 (M+H)⁺.

### Examples 29-35

The compounds in the table below were prepared from the appropriate starting materials, prepared as described above or commercially available, using an appropriate modification of General Procedure 6 and (R)-N-cyclopropyl-1-(4-methoxypyrimidin-2-yl)piperidin-3-amine (J1).

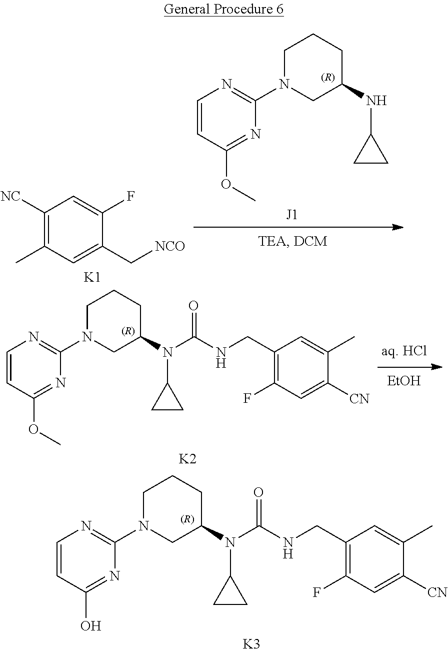

### Step 1: Synthesis of Compound K₂

To a mixture of compound K1 (50 mg, 0.305 mmol) and compound J1 (76 mg, 0.305 mmol) in anhydrous DCM (5 mL) was added TEA (92 mg, 0.914 mmol) and Triphosgene (46 mg, 0.152 mmol) at −40° C. under N₂ atmosphere. The resulting mixture was stirred at room temperature for 2 hrs under N₂ atmosphere. Then the mixture was diluted with DCM (20 mL) and washed with water (30 mL) and brine (30 mL). The organic layer was separated, dried over anhydrous Na₂SO₄ and concentrated to dryness. The residue was purified by column chromatography on silica gel (eluted with DCM:MeOH=100:0 to 95:5) to afford compound K2 (40 mg, 29.95% yield) as light-yellow solid. LC/MS (ESI) m/z: 439 (M+H)⁺.

### Step 2: Synthesis of Compound K3 (Example 29)

To a mixture of compound K2 (40 mg, 0.091 mmol) in EtOH (2 mL) was added conc. HCl (1 mL) and the resulting mixture was stirred at 80° C. for 2 hrs under N₂ atmosphere. After cooling, the mixture was diluted with water (10 mL) and basified with aq. NaHCO₃ in portions to adjust pH=8. Then the mixture was extracted with DCM (20 mL) twice. The combined organic layers were washed with water (30 mL) and brine (30 mL). The organic layer was separated, dried over anhydrous Na₂SO₄ and concentrated to dryness. The residue was purified via preparative HPLC to afford compound K3 (20 mg, 51.65% yield) as white solid. LC/MS (ESI) m/z: 425 (M+H)⁺. ¹H NMR (400 MHz, MeOD) δ 7.57 (d, J=11.0 Hz, 1H), 7.43 (d, J=9.6 Hz, 1H), 7.36 (d, J=7.1 Hz, 1H), 7.05 (t, J=5.9 Hz, 1H), 5.74 (d, J=6.4 Hz, 1H), 4.46 (d, J=5.4 Hz, 2H), 4.35-4.22 (m, 2H), 3.70 (t, J=11.7 Hz, 1H), 3.38-3.30 (m, 1H), 2.91-2.80 (m, 1H), 2.62-2.54 (m, 1H), 2.50 (s, 3H), 2.32-2.15 (m, 1H), 2.00-1.81 (m, 2H), 1.67-1.51 (m, 1H), 1.02-0.90 (m, 2H), 0.86-0.74 (m, 2H).

| Example | Structure and name | Data |
|---|---|---|
| (29) | 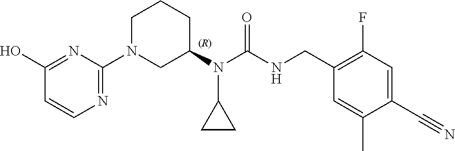 3-[(4-cyano-2-fluoro-5- | LC-MS: m/z = 425.3 (M + H) 1H NMR: (400 MHz, MeOD) δ 7.57 (d, J = 11.0 Hz, 1H), 7.43 (d, J = 9.6 Hz, 1H), 7.36 (d, J = 7.1 Hz, 1H), 7.05 (t, J = 5.9 Hz, 1H), 5.74 (d, J = 6.4 Hz, 1H), 4.46 (d, J = 5.4 Hz, 2H), 4.35 − 4.22 (m, 2H), 3.70 (t, J = 11.7 Hz, 1H), 3.38 − 3.32 (m, 1H), 2.91 − 2.80 (m, 1H), 2.62 − 2.54 (m, 1H), 2.50 (s, 3H), 2.32 − 2.15 (m, 1H), 2.00 − 1.81 (m, 2H), 1.67 − 1.51 (m, 1H), 1.02 − 0.91 (m, 2H), 0.85 − 0.76 (m, 2H). |
| | methylphenyl)methyl]-1- | |
| | cyclopropyl-1-[(3R)-1-(4- | |
| | hydroxypyrimidin-2-yl)piperidin-3- | |
| | yl]urea | |
| (30) | 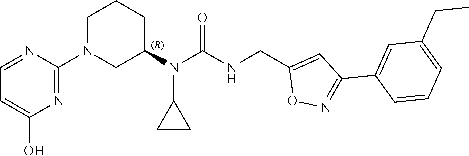 1-cyclopropyl-3-{[3-(3- ethylphenyl)-1,2-oxazol-5- yl]methyl}-1-[(3R)-1-(4- | LC-MS: m/z = 463.3 (M + H) 1H NMR: (400 MHz, MeOD) δ 7.66 (s, 1H), 7.61 (d, J = 7.6 Hz, 2H), 7.38 (t, J = 7.6 Hz, 1H), 7.31 (d, J = 7.7 Hz, 1H), 7.18 (t, J = 5.8 Hz, 1H), 6.65 (s, 1H), 5.73 (d, J = 6.7 Hz, 1H), 4.57 − 4.52 (m, 2H), 4.34 − 4.23 (m, 2H), 3.72 (tt, J = 11.6, 3.8 Hz, 1H), 3.36 (d, J = 12.1 Hz, 1H), 2.86 (td, J = 13.2, 2.6 Hz, 1H), 2.71 (q, J = 7.6 Hz, 2H), 2.60 − 2.54 (m, 1H), 2.22 (qd, J = 12.6, 4.2 Hz, 1H), 1.91 (dd, J = 44.6, 12.6 Hz, 2H), 1.66 − 1.54 (m, 1H), 1.27 (t, J = 7.6 Hz, 3H), 0.97 (dd, J = 6.5, 3.2 Hz, 2H), 0.86 − 0.79 (m, 2H). |
| | hydroxypyrimidin-2-yl)piperidin-3- | |
| | yl]urea | |
| (31) | 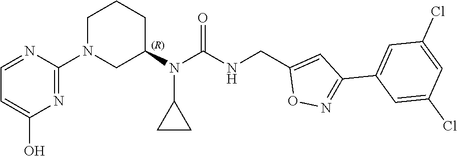 1-cyclopropyl-3-{[3-(3,5- | LC-MS: m/z = 503.2 (M + H) 1H NMR: (400 MHz, MeOD) δ 7.82 (d, J = 1.9 Hz, 2H), 7.56 (t, J = 1.9 Hz, 2H), 6.75 (s, 1H), 5.73 (d, J = 6.7 Hz, 1H), 4.55 (s, 2H), 4.29 (t, J = 13.6 Hz, 2H), 3.72 (t, J = 11.7 Hz, 1H), 3.35 (d, J = 12.3 Hz, 1H), 2.87 (t, J = 12.0 Hz, 1H), 2.61 − 2.51 (m, 1H), 2.22 (dd, J = 12.5, 3.9 Hz, 1H), 1.97 (d, J = 11.1 Hz, 1H), 1.85 (d, J = 13.4 Hz, 1H), 1.59 (dd, J = 9.3, 3.8 Hz, 1H), 0.98 (dd, J = 6.4, 3.3 Hz, 2H), 0.83 (d, J = 3.9 Hz, 2H). |
| | dichlorophenyl)-1,2-oxazol-5- | |
| | yl]methyl}-1-[(3R)-1-(4- | |
| | hydroxypyrimidin-2-yl)piperidin-3- | |
| | yl]urea | |
| (32) | 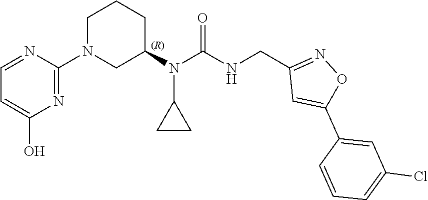 | LC-MS: m/z = 469.2 (M + H) 1H NMR: (400 MHz, MeOD) δ 7.83 (s, 1H), 7.76 (dd, J = 5.6, 2.8 Hz, 1H), 7.59 (d, J = 5.7 Hz, 1H), 7.52 − 7.45 (m, 2H), 6.80 (s, 1H), 5.74 (d, J = 6.7 Hz, 1H), 4.56 − 4.42 (m, 2H), 4.37 − 4.18 (m, 2H), 3.74 (ddd, J = 11.8, 8.0, 4.0 Hz, 1H), 3.36 (d, J = 12.0 Hz, 1H), 2.87 (dd, J = 13.1, 10.8 Hz, 1H), 2.63 − 2.50 (m, 1H), 2.22 (qd, J = 12.5, 4.0 Hz, 1H), 1.97 (d, J = 11.6 Hz, 1H), 1.86 (d, J = 13.1 Hz, 1H), 1.60 (dtd, J = 13.2, 9.3, 4.2 Hz, 1H), 0.97 (dt, J = 6.4, 3.2 Hz, 2H), 0.83 (dd, J = 6.3, 2.8 Hz, 2H). |
| | 3-{[5-(3-chlorophenyl)-1,2-oxazol- | |
| | 3-yl]methyl}-1-cyclopropyl-1-[(3R)- | |
| | 1-(4-hydroxypyrimidin-2- | |
| | yl)piperidin-3-yl]urea | |
| (33) | 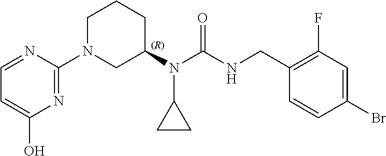 1-[(4-bromo-2-fluorophenyl)methyl]-3-cyclopropyl-3-[(3R)-1-(4- hydroxypyrimidin-2-yl)piperidin-3-yl]urea | LC-MS: m/z = 464.0 (M + H) 1H NMR: (400 MHz, dmso) δ 11.12 (s, 1H), 7.68 (s, 1H), 7.48 (dd, J = 9.8, 2.0 Hz, 1H), 7.40 (dd, J = 8.2, 1.9 Hz, 1H), 7.27 (t, J = 8.2 Hz, 1H), 6.91 (t, J = 5.9 Hz, 1H), 5.60 (d, J = 6.2 Hz, 1H), 4.37 (t, J = 13.3 Hz, 2H), 4.27 (d, J = 5.8 Hz, 2H), 3.52 (t, J = 11.7 Hz, 1H), 3.17 (t, J = 12.0 Hz, 1H), 2.74 − 2.63 (m, 1H), 2.46 (dt, J = 6.8, 3.7 Hz, 1H), 2.11 − 1.97 (m, 1H), 1.77 (d, J = 12.5 Hz, 1H), 1.70 (d, J = 13.6 Hz, 1H), 1.48 − 1.34 (m, 1H), 0.88 (td, J = 5.9, 3.3 Hz, 2H), 0.71 − 0.64 (m, 2H). |
| (34) | 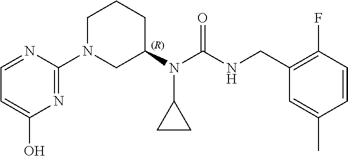 | LC-MS: m/z = 400.3 (M + H) 1H NMR: (400 MHz, dmso) δ 11.10 (s, 1H), 7.68 (s, 1H), 7.12 − 6.97 (m, 3H), 6.80 (t, J = 5.9 Hz, 1H), 5.60 (d, J = 6.3 Hz, 1H), 4.39 − 4.33 (m, 2H), 4.29 (d, J = 5.9 Hz, 2H), 3.54 (t, J = 11.6 Hz, 1H), 3.18 (t, J = 12.0 Hz, 1H), 2.69 (t, J = 12.8 Hz, 1H), 2.49 − 2.44 (m, 1H), 2.27 (s, 3H), 2.12 − 2.00 (m, 1H), 1.82 − 1.67 (m, 2H), 1.47 − 1.37 (m, 1H), 0.93 − 0.84 (m, 2H), 0.71 − 0.64 (m, 2H). |
| | 3-cyclopropyl-1-[(2-fluoro-5- | |
| | methylphenyl)methyl]-3-[(3R)-1-(4- | |
| | hydroxypyrimidin-2-yl)piperidin-3- | |
| | yl]urea | |
| (35) | 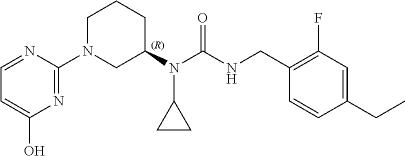 | LC-MS: m/z = 414.4 (M + H) |
| | 3-cyclopropyl-1-[(4-ethyl-2- | |
| | fluorophenyl)methyl]-3-[(3R)-1-(4- | |
| | hydroxypyrimidin-2-yl)piperidin-3- | |
| | yl]urea | |

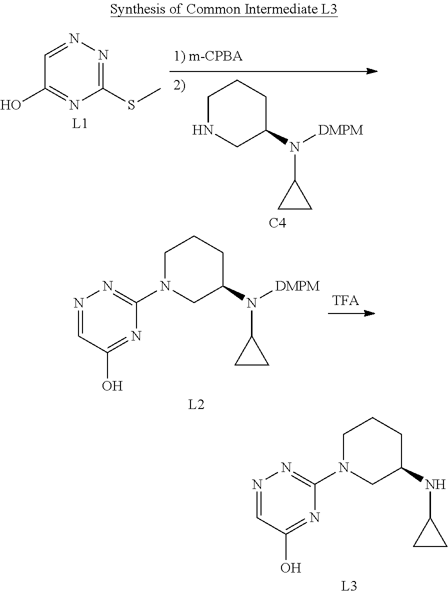

### Step 1: Synthesis of Compound L2

To a mixture of compound L1 (50 mg, 0.349 mmol) in anhydrous DCM (4 mL) was added m-CPBA (121 mg, 0.698 mmol) dropwise at 0° C. The resulting mixture was stirred at room temperature for 1 hr. Then a solution of compound C4 (122 mg, 0.419 mmol) in iPrOH (4 mL) and DIEA (90.28 mg, 0.698 mmol) were added into the above mixture. The resulting mixture was stirred at 90° C. for 36 hrs. After cooling, the mixture was concentrated under reduced pressure to dryness. The residue was diluted with EtOAc (20 mL) and washed with water and brine. The organic layer was separated, dried over anhydrous Na₂SO₄ and concentrated to dryness. The residue was purified by column chromatography on silica gel (eluted with DCM:MeOH=100:0 to 94:6) to afford compound L2 (68 mg, 51.6% yield) as yellow oil. LC/MS (ESI) m/z: 386 (M+H)⁺.

### Step 2: Synthesis of Compound L3

Compound L2 (68 mg, 0.175 mmol) was added into TFA (3 mL) in portions at 0° C. under N₂ atmosphere. Then the resulting mixture was stirred at 80° C. for 4 hrs. After cooling to room temperature, the mixture was concentrated under reduced pressure to give crude compound L3 (40.32 mg, 98.61% yield) as purple oil which was used at the next step directly without further purification. LC/MS (ESI) m/z: 236 (M+H)⁺.

### Examples 36-42

The compounds in the table below were prepared from the appropriate starting materials, prepared as described above or commercially available, using an appropriate modification of General Procedure 7 and (R)-3-(3-(cyclopropylamino)piperidin-1-yl)-1,2,4-triazin-5-ol (L3).

### General Procedure 7

### Synthesis of Compound L5

To a solution of compound L3 (41 mg, 0.171 mmol) in anhydrous DCM (3 mL) at 0° C. under N₂ atmosphere was added L4 (35 mg, 0.171 mmol) and TEA (52 mg, 0.513 mmol). The mixture was stirred at room temperature for 1 hr and then concentrated under reduced pressure to dryness. The residue was dissolved with EtOAc (20 mL) and washed with water and brine. The organic layer was separated, dried over anhydrous Na₂SO₄ and concentrated to dryness. The crude product was purified by column chromatography on silica gel (eluted with DCM:MeOH=100:0 to 95:5) to afford L5 (13 mg, 16.58% yield) as white solid. LC/MS (ESI) m/z: 435 (M+H)⁺. ¹H NMR (400 MHz, MeOD) δ 7.35 (s, 1H), 7.25 (d, J=8.0 Hz, 1H), 7.14 (d, J=9.8 Hz, 1H), 6.93 (s, 1H), 4.41-4.18 (m, 4H), 3.72-3.61 (m, 1H), 3.41 (t, J=12.2 Hz, 1H), 2.94-2.82 (m, 1H), 2.56 (s, 1H), 2.32 (s, 3H), 2.27-2.18 (m, 1H), 1.98-1.83 (m, 2H), 1.66-1.54 (m, 1H), 1.02-0.90 (m, 2H), 0.85-0.73 (m, 2H).

| Example | Structure and name | Data |
|---|---|---|
| (36) | 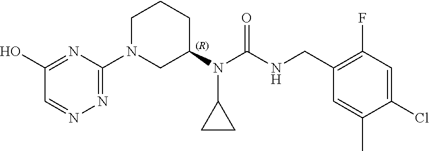 | LC-MS: m/z = 435.2 (M + H) 1H NMR: (400 MHz, MeOD) δ 7.35 (s, 1H), 7.25 (d, J = 8.0 Hz, 1H), 7.14 (d, J = 9.8 Hz, 1H), 6.93 (s, 1H), 4.41 − 4.18 (m, 4H), 3.72 − 3.61 (m, 1H), 3.41 (t, J = 12.2 Hz, 1H), 2.94 − 2.82 (m, 1H), 2.56 (s, 1H), 2.32 (s, 3H), 2.27 − 2.18 (m, 1H), 1.98 − 1.83 (m, 2H), 1.60 (d, J = 13.7 Hz, 1H), 0.96 (s, 2H), 0.77 (d, J = 3.6 Hz, 2H). |
| | 3-[(4-chloro-2-fluoro-5- | |
| | methylphenyl)methyl]-1-cyclopropyl- | |
| | 1-[(3R)-1-(5-hydroxy-1,2,4-triazin-3- | |
| | yl)piperidin-3-yl]urea | |
| (37) | 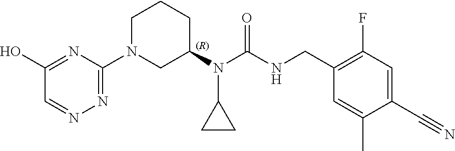 3-[(4-cyano-2-fluoro-5- | LC-MS: m/z = 426.3 (M + H) 1H NMR: (400 MHz, MeOD) δ 7.43 (d, J = 9.6 Hz, 1H), 7.34 (d, J = 4.0 Hz, 2H), 7.05 (t, J = 5.9 Hz, 1H), 4.45 (d, J = 4.5 Hz, 2H), 4.28 (t, J = 11.6 Hz, 2H), 3.66 (dd, J = 14.0, 9.7 Hz, 1H), 3.39 (d, J = 11.9 Hz, 1H), 2.88 (t, J = 12.0 Hz, 1H), 2.62 − 2.56 (m, 1H), 2.49 (s, 3H), 2.23 (tt, J = 12.6, 6.4 Hz, 1H), 1.91 (dd, J = 32.0, 12.6 Hz, 2H), 1.61 (t, J = 13.2 Hz, 1H), 1.00 − 0.93 (m, 2H), 0.80 (d, J = 3.4 Hz, 2H). |
| | methylphenyl)methyl]-1-cyclopropyl- | |
| | 1-[(3R)-1-(5-hydroxy-1,2,4-triazin-3- | |
| | yl)piperidin-3-yl]urea | |
| (38) | 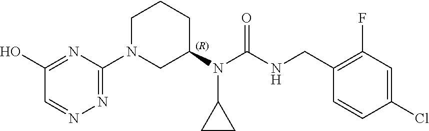 | LC-MS: m/z = 421.1 (M + H) 1H NMR: (400 MHz, MeOD) δ 7.39 − 7.31 (m, 2H), 7.23 − 7.15 (m, 2H), 4.48 − 4.37 (m, 2H), 4.37 − 4.22 (m, 2H), 3.69 (t, J = 11.6 Hz, 1H), 3.42 (t, J = 12.1 Hz, 1H), 2.90 (t, J = 12.7 Hz, 1H), 2.58 (d, J = 3.5 Hz, 1H), 2.34 − 2.17 (m, 1H), 2.02 − 1.81 (m, 2H), 1.70 − 1.51 (m, 1H), 0.97 (s, 2H), 0.80 (d, J = 3.2 Hz, 2H). |
| | 3-[(4-chloro-2-fluorophenyl)methyl]- | |
| | 1-cyclopropyl-1-[(3R)-1-(5-hydroxy- | |
| | 1,2,4-triazin-3-yl)piperidin-3-yl]urea | |
| (39) | 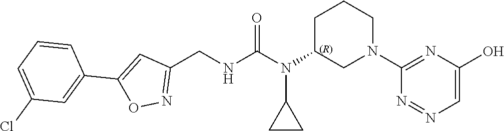 3-{[5-(3-chlorophenyl)-1,2-oxazol-3- yl]methyl}-1-cyclopropyl-1-[(3R)-1- | LC-MS: m/z = 470.2 (M + H) 1H NMR: (400 MHz, MeOD) δ 7.84 (s, 1H), 7.76 (dd, J = 5.6, 2.6 Hz, 1H), 7.50 (dd, J = 8.4, 5.5 Hz, 2H), 7.37 (d, J = 2.1 Hz, 1H), 6.80 (s, 1H), 4.49 (s, 2H), 4.37 − 4.25 (m, 2H), 3.78 − 3.66 (m, 1H), 3.49 − 3.37 (m, 1H), 2.96 − 2.85 (m, 1H), 2.63 − 2.53 (m, 1H), 2.30 − 2.19 (m, 1H), 1.98 (d, J = 10.1 Hz, 1H), 1.88 (d, J = 13.2 Hz, 1H), 1.67 − 1.55 (m, 1H), 1.01 − 0.94 (m, 2H), 0.87 − 0.79 (m, 2H). |
| | (5-hydroxy-1,2,4-triazin-3- | |
| | yl)piperidin-3-yl]urea | |
| (40) | 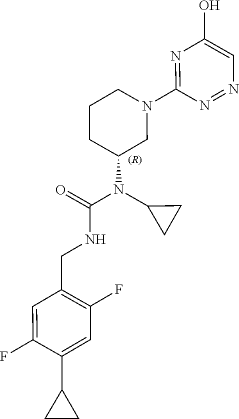 | LC-MS: m/z = 445.2 (M + H) 1H NMR: (400 MHz, MeOD) δ 7.35 (s, 1H), 6.96 (dt, J = 15.4, 7.7 Hz, 2H), 6.66 (dd, J = 10.8, 6.2 Hz, 1H), 4.34 (d, J = 17.2 Hz, 3H), 4.24 (d, J = 12.7 Hz, 1H), 3.71 − 3.63 (m, 1H), 3.41 (t, J = 12.1 Hz, 1H), 2.87 (t, J = 12.0 Hz, 1H), 2.60 − 2.54 (m, 1H), 2.28 − 2.18 (m, 1H), 2.08 − 2.02 (m, 1H), 1.97 − 1.92 (m, 1H), 1.87 (d, J = 14.1 Hz, 1H), 1.64 − 1.55 (m, 1H), 1.01 − 0.94 (m, 4H), 0.80 − 0.75 (m, 2H), 0.70 (dd, J = 5.0, 1.6 Hz, 2H). |
| | 1-cyclopropyl-3-[(4-cyclopropyl-2,5- | |
| | difluorophenyl)methyl]-1-[(3R)-1-(5- | |
| | hydroxy-1,2,4-triazin-3-yl)piperidin- | |
| | 3-yl]urea | |
| (41) | 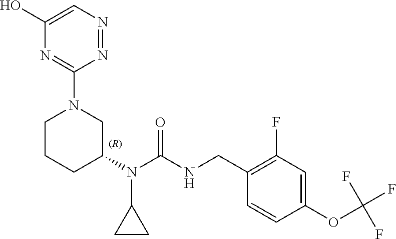 | LC-MS: m/z = 471.2 (M + H) 1H NMR: (400 MHz, MeOD) δ 7.44 (t, J = 8.6 Hz, 1H), 7.35 (s, 1H), 7.10 (t, J = 7.9 Hz, 2H), 4.44 (s, 2H), 4.36 − 4.20 (m, 2H), 3.67 (t, J = 11.7 Hz, 1H), 3.41 (t, J = 12.1 Hz, 1H), 2.88 (t, J = 11.8 Hz, 1H), 2.58 (d, J = 3.4 Hz, 1H), 2.32 − 2.17 (m, 1H), 2.00 − 1.81 (m, 2H), 1.59 (d, J = 12.8 Hz, 1H), 0.97 (d, J = 5.1 Hz, 2H), 0.79 (s, 2H). |
| | 3-cyclopropyl-1-{[2-fluoro-4- | |
| | (trifluoromethoxy)phenyl]methyl}-3- | |
| | [(3R)-1-(5-hydroxy-1,2,4-triazin-3- | |
| | yl)piperidin-3-yl]urea | |
| (42) | 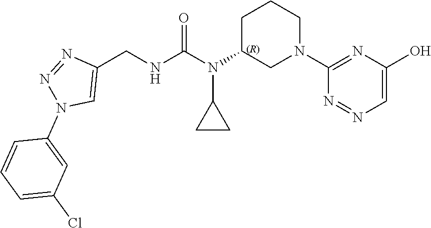 | LC-MS: m/z = 470.2 (M + H) 1H NMR: (400 MHz, MeOD) δ 8.41 (s, 1H), 7.95 (t, J = 1.9 Hz, 1H), 7.82 (d, J = 8.0 Hz, 1H), 7.57 (t, J = 8.0 Hz, 1H), 7.50 (d, J = 8.1 Hz, 1H), 7.35 (s, 1H), 4.53 (d, J = 4.1 Hz, 2H), 4.38 − 4.20 (m, 2H), 3.78 − 3.65 (m, 1H), 3.40 (t, J = 12.1 Hz, 1H), 2.96 − 2.83 (m, 1H), 2.60 − 2.51 (m, 1H), 2.32 − 2.18 (m, 1H), 1.92 (dd, J = 35.5, 13.0 Hz, 2H), 1.67 − 1.52 (m, 1H), 1.03 − 0.92 (m, 2H), 0.89 − 0.75 (m, 2H). |
| | 3-{[1-(3-chlorophenyl)-1H-1,2,3- | |
| | triazol-4-yl]methyl}-1-cyclopropyl-1- | |
| | [(3R)-1-(5-hydroxy-1,2,4-triazin-3- | |
| | yl)piperidin-3-yl]urea | |

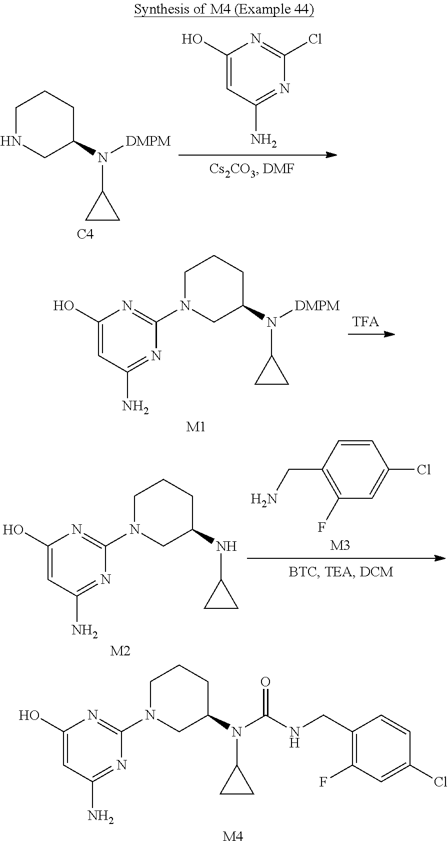

### Step 1: Synthesis of Compound M1

To a mixture of compound C4 (100 mg, 0.344 mmol) and Cs₂CO₃ (337 mg, 1.03 mmol) in anhydrous DMF (8 mL) was added 6-amino-2-chloro-1H-pyrimidin-4-one (101 mg, 0.689 mmol). The resulting mixture was stirred at 120° C. for 3 hrs under microwave. Then the mixture was cooled, diluted with EtOAc (30 mL) and washed with saturated NH₄Cl solution (30 mL×3) and brine (20 mL). The organic layer was separated, dried over anhydrous Na₂SO₄ and concentrated to dryness. The crude product was purified by column chromatography on silica gel (eluted with DCM:MeOH=100:0 to 90:10) to afford compound M1 (112 mg, 81.4% yield) as light-yellow solid. LC/MS (ESI) m/z: 400 (M+H)⁺.

### Step 2: Synthesis of Compound M2

Compound M1 (112.0 g, 0.280 mmol) was added into TFA (6 mL) in portions at 0° C. under N₂ atmosphere. Then the resulting mixture was stirred at 80° C. for 4 hrs. After cooling to room temperature, the mixture was concentrated under reduced pressure to give crude compound M2 (69 mg, 98.72% yield) as purple oil which was used at the next step directly without further purification. LC/MS (ESI) m/z: 250 (M+H)⁺.

### Step 3: Synthesis of Compound M4

To a mixture of compound M2 (69 mg, 0.277 mmol), TEA (84 mg, 0.830 mmol) and M3 (44.17 mg, 0.277 mmol) in anhydrous DCM (10 mL) was added triphosgene (41.06 mg, 0.138 mmol) at −50° C. under N₂ atmosphere. The resulting mixture was allowed to warm to room temperature slowly and stirred for another 2 hrs. Then the mixture was concentrated under reduced pressure to dryness. The residue was dissolved with EtOAc (20 mL) and washed with water (20 mL) and brine (20 mL). The organic layer was separated, dried over anhydrous Na₂SO₄ and concentrated to dryness. The crude product was purified by column chromatography on silica gel (eluted with DCM:MeOH=100:0 to 95:5) to afford M4 (15.0 mg, 12.46% yield) as white solid.

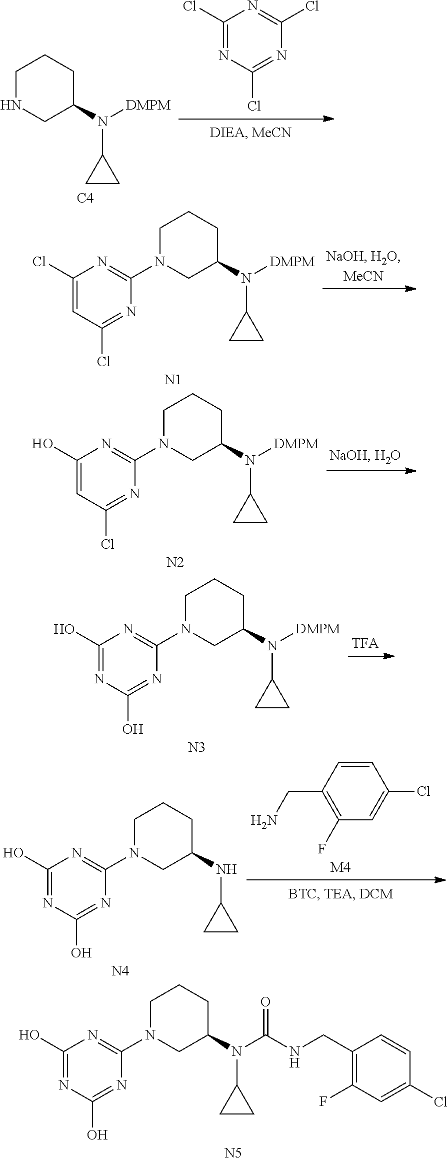

### Synthesis of N5 (Example 45)

### Step 1: Synthesis of Compound N1

To a mixture of compound C4 (1 g, 3.448 mmol) and DIEA (1.33 g, 10.345 mmol) in anhydrous MeCN (20 mL) was added 2,4,6-trichloro-1,3,5-triazine (635 mg, 3.448 mmol) dropwise at 0° C. under N₂ atmosphere. The resulting mixture was stirred at room temperature for 1 hr. Then the mixture was concentrated under reduced pressure to dryness. The residue was diluted with EtOAc (60 mL) and washed with water (50 mL) and brine (50 mL). The organic layer was separated, dried over anhydrous Na₂SO₄ and concentrated to dryness. The residue was purified by column chromatography on silica gel (eluted with PE:EA=100:0 to 90:10) to afford compound L1 (1.3 g, 86.6% yield) as light-yellow solid. LC/MS (ESI) m/z: 437 (M+H)⁺.

### Step 2: Synthesis of Compound N2

To a mixture of compound N1 (1.3 g, 2.294 mmol) in MeCN (15 mL) was added aq. NaOH (15 mL, 1 N). The resulting mixture was stirred at room temperature for 12 hrs. Then the mixture was diluted with EtOAc (50 mL) and washed with water (50 mL) and brine (50 mL). The organic layer was separated, dried over anhydrous Na₂SO₄ and concentrated to dryness. The crude product was purified by column chromatography on silica gel (eluted with DCM:MeOH=100:0 to 90:10) to afford compound N₂ (802 mg, 64.5% yield) as colorless oil. LC/MS (ESI) m/z: 419 (M+H)⁺.

### Step 3: Synthesis of Compound N3

To a mixture of compound N2 (802 mg, 1.914 mmol) in MeCN (10 mL) was added aq. NaOH (10 mL, 6 N). The resulting mixture was stirred at 100° C. for 12 hrs. After cooling, the mixture was diluted with EtOAc (50 mL) and washed with water (50 mL) and brine (50 mL). The organic layer was separated, dried over anhydrous Na₂SO₄ and concentrated to dryness. The crude product was purified by column chromatography on silica gel (eluted with DCM:MeOH=100:0 to 90:10) to afford compound N3 (370 mg, 48.2% yield) as colorless oil. LC/MS (ESI) m/z: 402 (M+H)⁺.

### Step 4: Synthesis of Compound N4

Compound N3 (370 mg, 0.92 mmol) was added into TFA (10 mL) in portions at 0° C. under N₂ atmosphere. Then the resulting mixture was stirred at 80° C. for 4 hrs. After cooling to room temperature, the mixture was concentrated under reduced pressure to give crude compound N4 (200 mg, 86.9% yield) as purple oil which was used at the next step directly without further purification. LC/MS (ESI) m/z: 252 (M+H)⁺.

### Step 5: Synthesis of Compound N5

To a mixture of M4 (80 mg, 0.503 mmol), TEA (254 mg, 2.52 mmol) and compound N4 (152 mg, 0.604 mmol) in anhydrous DCM (10 mL) at −50° C. under N₂ atmosphere was added a solution of BTC (90 mg, 0.302 mmol) in DCM (5 mL) drop-wise. The resulting mixture was stirred at room temperature for 2 hrs and then concentrated under reduced pressure to dryness. The residue was dissolved with EtOAc (20 mL) and washed with water (20 mL) and brine (20 mL). The organic layer was separated, dried over anhydrous Na₂SO₄ and concentrated to dryness. The crude product was purified by column chromatography on silica gel (eluted with DCM:MeOH=100:0 to 95:5) to afford compound L5 (45 mg, 20.3% yield) as white solid.

### Examples 43-44

The compounds in the table below were prepared from (R)-N-cyclopropyl-N-(3,4-dimethoxybenzyl)piperidin-3-amine (C₄) according the above procedures.

| | | |
|---|---|---|
| (43) | 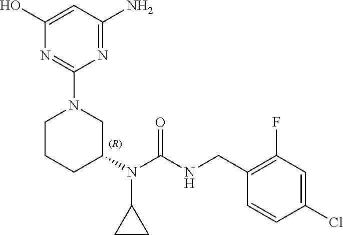 | LC-MS: m/z = 435.2 (M + H) 1H NMR: 1H NMR (400 MHz, MeOD) δ 7.33 (t, J = 8.3 Hz, 1H), 7.22 − 7.13 (m, 2H), 6.97 (t, J = 5.9 Hz, 1H), 4.49 − 4.36 (m, 2H), 4.12 (dd, J = 38.7, 11.8 Hz, 2H), 3.73 (td, J = 11.7, 3.9 Hz, 1H), 3.28 − 3.21 (m, 1H), 2.86 (td, J = 13.2, 2.3 Hz, 1H), 2.56 − 2.49 (m, 1H), 2.21 (qd, J = 12.7, 4.3 Hz, 1H), 1.96 (d, J = 12.0 Hz, 1H), 1.83 (d, J = 13.2 Hz, 1H), 1.63 − 1.49 (m, 1H), 1.01 − 0.91 (m, 2H), 0.82 − 0.70 (m, 2H). |
| | 3-[(3R)-1-(4-amino-6- | |
| | hydroxypyrimidin-2-yl)piperidin-3-yl]- | |
| | 1-[(4-chloro-2-fluorophenyl)methyl]-3- | |
| | cyclopropylurea | |
| (44) | 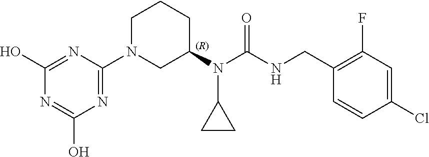 | LC-MS: m/z = 437.2 (M + H) 1H NMR: 1H NMR (400 MHz, DMSO) δ 10.96 (s, 1H), 10.50 (s, 1H), 7.42 − 7.18 (m, 3H), 6.95 (t, J = 5.9 Hz, 1H), 4.29 (d, J = 5.3 Hz, 3H), 3.50 (t, J = 11.6 Hz, 1H), 3.26 (s, 1H), 2.81 − 2.64 (m, 1H), 2.52 (s, 1H), 2.48 − 2.44 (m, 1H), 2.13 − 1.98 (m, 1H), 1.82 − 1.66 (m, 2H), 1.42 (d, J = 13.2 Hz, 1H), 0.93 − 0.83 (m, 2H), 0.74 − 0.60 (m, 2H). |
| | 3-[(4-chloro-2-fluorophenyl)methyl]-1- | |
| | cyclopropyl-1-[(3R)-1-(4,6-dihydroxy- | |
| | 1,3,5-triazin-2-yl)piperidin-3-yl]urea | |

### Synthesis of Common Intermediate O4

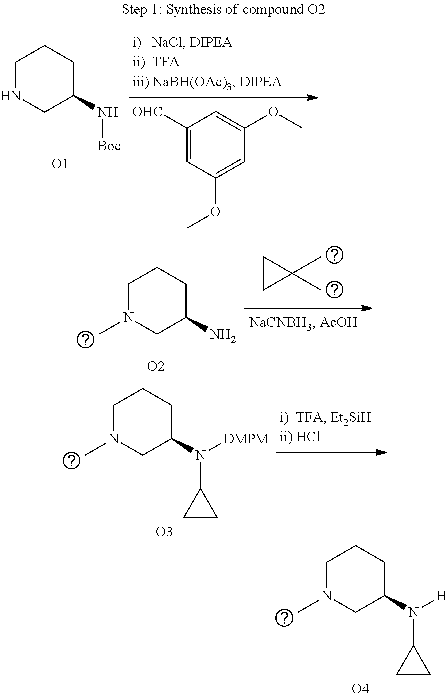

To a solution of tert-butyl N-[(3R)-3-piperidyl]carbamate (10 g, 49.93 mmol) 01 and DIPEA (7.10 g, 54.92 mmol, 9.57 mL) in DCM (500 mL) was added 2-nitrobenzenesulfonyl chloride (11.62 g, 52.43 mmol) as a solid, portionwise. The reaction mixture was allowed to stir for 15 minutes at ambient temperature and then TFA (56.93 g, 499.31 mmol, 38.47 mL) was slowly added. The reaction mixture was stirred at ambient temperature for an additional two hours and then concentrated under reduced pressure by rotary evaporation. The crude residue was then resuspended in 500 mL of DCM, and to this solution was added DIPEA (11.29 g, 87.38 mmol, 15.22 mL), 2,4-dimethoxybenzaldehyde (7.88 g, 47.43 mmol), and Sodium triacetoxyborohydride (26.46 g, 124.83 mmol). The reaction mixture was allowed to stir at ambient temperature overnight. The reaction mixture was then washed with 1M NaOH (500 mL) and then the organic layer was separated, dried with anhydrous sodium sulfate, and concentrated under reduced pressure by rotary evaporation. The crude reside was purified by flash column chromatography (DCM:MeOH, 100:0 to 96:4) to provide (3R)-N-[(2,4-dimethoxyphenyl)methyl]-1-(2-nitrophenyl)sulfonyl-piperidin-3-amine (7.94 g, 18.23 mmol, 36.52% yield) O2.

### Step 1: Synthesis of Compound O3

To a solution of (3R)-N-[(2,4-dimethoxyphenyl)methyl]-1-(2-nitrophenyl)sulfonyl-piperidin-3-amine (7.93 g, 18.21 mmol) O2 in THE (240 mL) and EtOH (120 mL) was added 1-ethoxy-1-trimethylsiloxycyclopropane (7.94 g, 45.52 mmol, 9.15 mL), Sodium cyanoborohydride (4.01 g, 63.73 mmol), and acetic acid (16.40 g, 273.14 mmol, 15.62 mL). The reaction mixture was stirred at 80 C overnight, after which it was cooled to rt, and concentrated under reduced pressure by rotary evaporation. The residue was then dissolved in ethyl acetate (250 mL) and washed with 1M NaOH (250 mL), brine (250 mL), dried with Na₂SO₄, filtered and concentrated under reduced pressure by rotary evaporation to provide (3R)-N-cyclopropyl-N-[(2,4-dimethoxyphenyl)methyl]-1-(2-nitrophenyl)sulfonyl-piperidin-3-amine (8.58 g, 18.04 mmol, 99.08% yield) O3 which was used without further purification.

### Step 1: Synthesis of Compound O4

(3R)-N-cyclopropyl-N-[(2,4-dimethoxyphenyl)methyl]-1-(2-nitrophenyl)sulfonyl-piperidin-3-amine (8.58 g, 18.04 mmol) was dissolved in TFA (100 mL) O3 and Et₃SiH (10 mL) and heated to 80 C. After four hours an additional portion of Triethylsilane (7.28 g, 62.61 mmol, 10 mL) was added to suppress the formation of the dimethoxytolyl cation. The reaction mixture was then allowed to stir overnight. The reaction mixture was then concentrated under reduced pressure by rotary evaporation and the crude residue was dissolved in 200 mL of ethyl acetate. The organic layer was washed with 3M NaOH (˜200 mL), brine (˜200 mL) dried with Na₂SO₄, filtered and concentrated under reduced pressure by rotary evaporation. The crude residue was then dissolved in 100 mL diethyl ether and HCl (2.0 M in diethyl ether, 9.02 mL) was slowly added dropwise. The product was filtered from the solution to provide (3R)-N-cyclopropyl-1-(2-nitrophenyl)sulfonyl-piperidin-3-amine (5.5 g, 15.20 mmol, 84.25% yield, HCl salt) 04 as a tan solid.

### Examples 45-48

The compounds in the table below were prepared from the appropriate starting materials, prepared as described above or commercially available, using an appropriate modification of General Procedure 8 and ((3R)-N-cyclopropyl-1-(2-nitrophenyl)sulfonyl-piperidin-3-amine (04).

### General Procedure 8

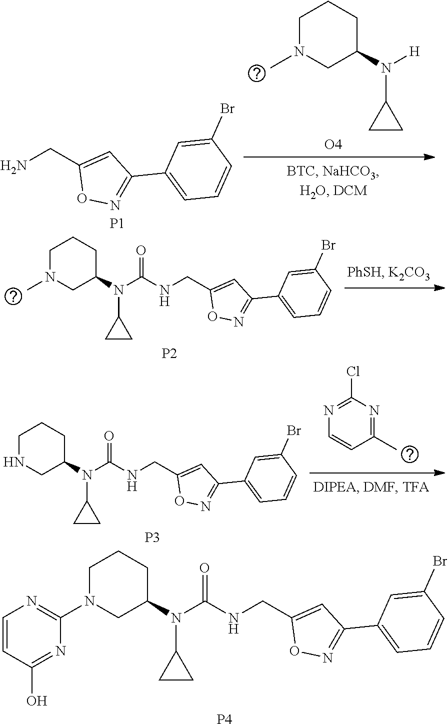

### Step 1: Synthesis of Compound P2

To a vigorously stirred solution of P1 (183.28 mg, 506.52 mol) in water (3 mL) and DCM (3 mL) was added sodium bicarbonate (255.32 mg, 3.04 mmol). The solution was cooled to OC then triphosgene (82.67 mg, 278.59 mol) was added as a single portion. The solution was stirred for one hour when 04 (183.28 mg, 506.52 mol) was added in a single portion. The solution was then diluted with water and DCM. The phases were separated and the aqueous phase was extracted twice more with DCM. The combined organic phases were washed with brine, dried over MgSO4, and concentrated to a residue. The residue was purified by silica column chromatography (30-100% EtOAc/Heptane). P2 (179.5 mg, 296.95 mol, 58.63% yield) was isolated as a colorless oil.

### Step 2: Synthesis of Compound P3

To a solution of P2 (180 mg, 297.78 mol) in DMF (1 mL) was added potassium carbonate (82.31 mg, 595.56 mol) and thiophenol (49.21 mg, 446.67 mol, 45.78 μL). The resultant solution was stirred at room temperature for two hours then diluted with water (4 mL) and acidified with HCl (12 M, 248.15 L). The resultant suspension was extracted with toluene (3×5 mL) and the organic phases discarded. The aqueous phase was basified with NaOH (6 M, 1.49 mL) and then extracted with EtOAc (4×5 mL). The combined organic phases were washed with brine, dried over MgSO4, and concentrated to afford P3 (127 mg, 302.88 mol, 100%) as a yellow oil, which was used without further purification.

### Step 1: Synthesis of Compound P4 (Example 46)

To a solution of P3 (25 mg, 59.62 umol) in DMF (250 uL) was added DIPEA (21 uL, 120 umol) and 4-tert-butoxy-2-chloro-pyrimidine (14.5 mg, 77 umol). The solution was heated at 60 C for one hour at which time LCMS indicated consumption of starting material. The solution was concentrated in vacuo to remove DMF, then TFA (500 uL) was added. The solution was stirred at room temperature for one hour, at which time LCMS indicated cleavage of the t-butyl protection. The solution was concentrated again, then taken up in methanol and purified by preparative reverse phase HPLC.

| | | |
|---|---|---|
| (45) | 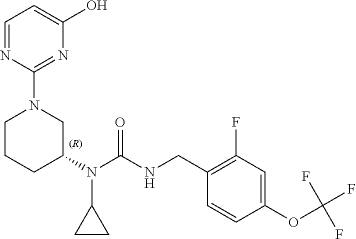 | LC-MS: m/z = 470.4 (M + H) 1H NMR: ¹H NMR (400 MHz, dmso) δ 7.68 (s, 1H), 7.43 (t, J = 8.5 Hz, 1H), 7.32 (dd, J = 10.3, 2.4 Hz, 1H), 7.23 (d, J = 8.6 Hz, 1H), 6.94 (t, J = 5.9 Hz, 1H), 5.60 (d, J = 6.2 Hz, 1H), 4.34 (t, J = 9.1 Hz, 4H), 3.53 (ddd, J = 11.8, 7.9, 4.1 Hz, 1H), 3.18 (t, J = 12.0 Hz, 1H), 2.75 − 2.63 (m, 1H), 2.50 − 2.43 (m, 5H), 2.05 (qd, J = 12.4, 4.0 Hz, 1H), 1.81 − 1.67 (m, 2H), 0.94 − 0.81 (m, 2H), 0.73 − 0.63 (m, 2H). |
| | 3-cyclopropyl-1-{[2-fluoro-4- | |
| | (trifluoromethoxy)phenyl]methyl}-3- | |
| | [(3R)-1-(4-hydroxypyrimidin-2- | |
| | yl)piperidin-3-yl]urea | |
| (46) | 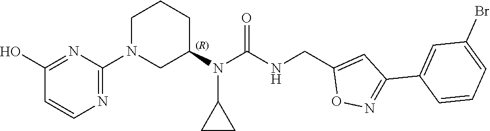 3-{[3-(3-bromophenyl)-1,2-oxazol-5- yl]methyl}-1-cyclopropyl-1-[(3R)-1- (4-hydroxypyrimidin-2-yl)piperidin-3- yl]urea | LC-MS: m/z = 513.3 (M + H) 1H NMR: ¹H NMR (400 MHz, dmso) δ 8.04 (t, J = 1.8 Hz, 1H), 7.88 (dt, J = 7.8, 1.3 Hz, 1H), 7.68 (ddd, J = 8.1, 2.2, 1.0 Hz, 1H), 7.46 (t, J = 7.9 Hz, 1H), 7.05 (t, J = 5.9 Hz, 1H), 6.86 (s, 1H), 5.62 (s, 1H), 4.43 (d, J = 5.8 Hz, 2H), 4.35 (d, J = 12.8 Hz, 2H), 3.61 − 3.49 (m, 1H), 3.18 (t, J = 11.9 Hz, 1H), 2.75 − 2.64 (m, 1H), 2.47 − 2.43 (m, 1H), 2.05 (qd, J = 12.5, 4.0 Hz, 1H), 1.79 (d, J = 12.3 Hz, 1H), 1.74 − 1.66 (m, 1H), 1.41 (tdd, J = 13.0, 9.1, 4.0 Hz, 1H), 0.88 (dq, J = 9.2, 2.7 Hz, 2H), 0.72 (h, J = 4.9 Hz, 2H). |
| (47) | 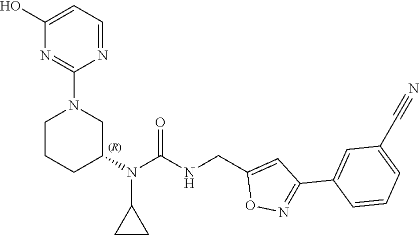 | LC-MS: m/z = 460.4 (M + H) 1H NMR: ¹H NMR (400 MHz, dmso) δ 11.11 (s, 1H), 8.34 (t, J = 1.7 Hz, 1H), 8.21 (dt, J = 8.0, 1.5 Hz, 1H), 7.95 (dt, J = 7.8, 1.4 Hz, 1H), 7.71 (t, J = 7.8 Hz, 1H), 7.66 (s, 1H), 7.06 (t, J = 5.8 Hz, 1H), 6.91 (s, 1H), 5.60 (s, 1H), 4.44 (d, J = 5.7 Hz, 2H), 4.35 (d, J = 12.4 Hz, 2H), 3.66 − 3.50 (m, 1H), 3.17 (t, J = 11.9 Hz, 1H), 2.77 − 2.63 (m, 1H), 2.47 − 2.43 (m, 1H), 2.05 (qd, J = 12.5, 4.1 Hz, 1H), 1.79 (d, J = 12.4 Hz, 1H), 1.70 (d, J = 13.4 Hz, 1H), 1.49 − 1.33 (m, 1H), 0.89 (dq, J = 9.0, 2.7 Hz, 2H), 0.73 (h, J = 3.1 Hz, 2H). |
| | 3-{[3-(3-cyanophenyl)-1,2-oxazol-5- | |
| | yl]methyl}-1-cyclopropyl-1-[(3R)-1- | |
| | (4-hydroxypyrimidin-2-yl)piperidin-3- | |
| | yl]urea | |
| (48) | 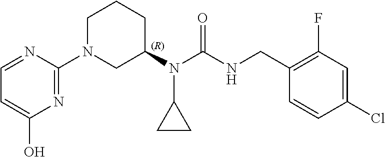 1-[(4-chloro-2-fluorophenyl)methyl]-3- cyclopropyl-3-[(3R)-1-(4- hydroxypyrimidin-2-yl)piperidin-3-yl]urea | LC-MS: m/z = 420.3 (M + H) 1H NMR: ¹H NMR (400 MHz, dmso) δ 11.12 (s, 1H), 7.68 (s, 1H), 7.40 − 7.29 (m, 2H), 7.27 (dd, J = 8.3, 2.1 Hz, 1H), 6.91 (t, J = 5.9 Hz, 1H), 5.60 (d, J = 6.3 Hz, 1H), 4.36 (t, J = 11.9 Hz, 2H), 4.29 (d, J = 5.8 Hz, 2H), 3.58 − 3.47 (m, 1H), 3.17 (t, J = 11.9 Hz, 1H), 2.74 − 2.63 (m, 1H), 2.46 (dt, J = 6.8, 3.6 Hz, 1H), 2.05 (qd, J = 12.5, 4.0 Hz, 1H), 1.77 (d, J = 12.3 Hz, 1H), 1.70 (d, J = 13.3 Hz, 1H), 1.48 − 1.37 (m, 1H), 0.88 (tt, J = 6.9, 3.0 Hz, 2H), 0.72 − 0.64 (m, 2H). |

### Examples 49-60

The compounds in the table below were prepared from the appropriate starting materials, prepared as described above, commercially available, or according to General Procedure 8 by using an appropriate modification of General Procedure 9 and ((3R)-N-cyclopropyl-1-(2-nitrophenyl)sulfonyl-piperidin-3-amine (04).

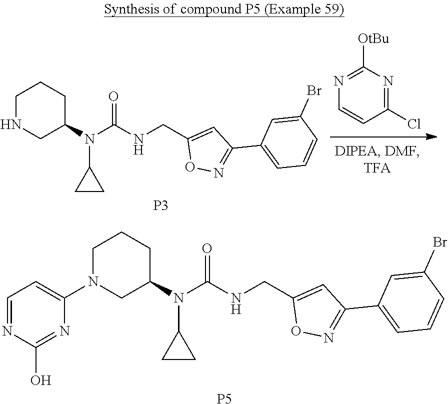

### General Procedure 9

To a solution of P3 (15 mg, 35.77 umol) in DMF (250 uL) was added DIPEA (13 uL, 72 umol) and 4-tert-butoxy-2-chloro-pyrimidine (10 mg, 47 umol). The solution was heated at 60 C for one hour at which time LCMS indicated consumption of starting material. The solution was concentrated in vacuo to remove DMF, then TFA (500 uL) was added. The solution was stirred at room temperature for one hour, at which time LCMS indicated cleavage of the t-butyl protection. The solution was concentrated again, then taken up in methanol and purified by preparative reverse phase HPLC.

| Example | Structure and name | Data |
|---|---|---|
| (49) | 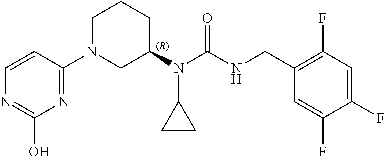 | LC-MS: m/z = 422.4 (M + H) |
| | 3-cyclopropyl-3-[(3R)-1-(2- | |
| | hydroxypyrimidin-4-yl)piperidin-3- | |
| | yl]-1-[(2,4,5- | |
| | trifluorophenyl)methyl]urea | |
| (50) | 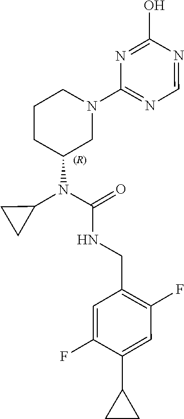 | LC-MS: m/z = 444.4 (M + H) 1H NMR: ¹H NMR (400 MHz, dmso) δ 10.42 (s, 1H), 7.40 (t, J = 6.0 Hz, 1H), 7.01 (dd, J = 10.6, 6.3 Hz, 1H), 6.89 (t, J = 5.9 Hz, 1H), 6.79 (dd, J = 10.8, 6.3 Hz, 1H), 6.58 (s, 0H), 5.99 (d, J = 7.5 Hz, 1H), 5.75 (s, 0H), 4.25 (d, J = 5.9 Hz, 2H), 3.50 (t, J = 12.0 Hz, 1H), 2.54 (s, 1H), 2.14 − 2.05 (m, 1H), 1.99 (dt, J = 8.8, 5.1 Hz, 1H), 1.82 (s, 1H), 1.74 (d, J = 13.2 Hz, 1H), 1.37 (d, J = 13.3 Hz, 1H), 1.01 − 0.92 (m, 2H), 0.89 (d, J = 6.7 Hz, 2H), 0.77 − 0.70 (m, 2H), 0.68 (s, 2H). |
| | 3-cyclopropyl-1-[(4-cyclopropyl-2,5- | |
| | difluorophenyl)methyl]-3-[(3R)-1-(2- | |
| | hydroxypyrimidin-4-yl)piperidin-3- | |
| | yl]urea | |
| (51) | 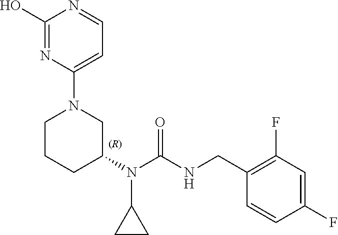 | LC-MS: m/z = 404.0 (M + H) 1H NMR: ¹H NMR (400 MHz, dmso) δ 10.43 (s, 1H), 7.37 (ddd, J = 15.5, 10.4, 7.0 Hz, 2H), 7.17 (ddd, J = 10.6, 9.3, 2.6 Hz, 1H), 7.07 (ddd, J = 11.0, 8.1, 2.4 Hz, 1H), 6.90 (t, J = 5.9 Hz, 1H), 5.99 (d, J = 7.5 Hz, 1H), 4.30 (d, J = 5.8 Hz, 2H), 3.57 − 3.47 (m, 1H), 2.51 − 2.42 (m, 8H), 2.08 (td, J = 12.4, 4.0 Hz, 1H), 1.84 − 1.70 (m, 2H), 1.44 − 1.30 (m, 1H), 0.89 (d, J = 6.7 Hz, 2H), 0.68 (s, 2H). |
| | 3-cyclopropyl-1-[(2,4- | |
| | difluorophenyl)methyl]-3-[(3R)-1-(2- | |
| | hydroxypyrimidin-4-yl)piperidin-3- | |
| | yl]urea | |
| (52) | 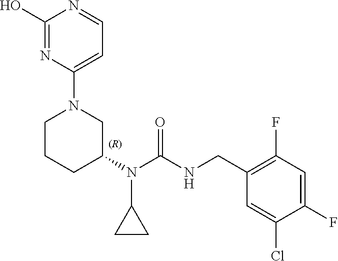 | LC-MS: m/z = 438.3 (M + H) 1H NMR: ¹H NMR (400 MHz, dmso) δ 10.42 (s, 1H), 7.52 − 7.43 (m, 2H), 7.40 (d, J = 7.1 Hz, 1H), 6.96 (t, J = 5.9 Hz, 1H), 5.98 (d, J = 7.4 Hz, 1H), 4.28 (d, J = 5.9 Hz, 2H), 3.50 (t, J = 11.8 Hz, 1H), 2.15 − 2.02 (m, 1H), 1.84 − 1.69 (m, 2H), 1.38 (t, J = 12.8 Hz, 1H), 0.88 (d, J = 6.7 Hz, 2H), 0.67 (s, 2H). |
| | 1-[(5-chloro-2,4- | |
| | difluorophenyl)methyl]-3- | |
| | cyclopropyl-3-[(3R)-1-(2- | |
| | hydroxypyrimidin-4-yl)piperidin-3- | |
| | yl]urea | |
| (53) | 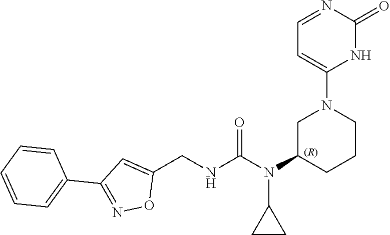 | LC-MS: m/z = 435.4 (M + H) 1H NMR: ¹H NMR (400 MHz, dmso) δ 10.41 (s, 1H), 7.89 − 7.81 (m, 2H), 7.54 − 7.43 (m, 3H), 7.42 − 7.34 (m, 1H), 7.06 (t, J = 5.8 Hz, 1H), 6.77 (s, 1H), 5.98 (d, J = 7.4 Hz, 1H), 4.63 (br, 1H), 4.43 (d, J = 5.8 Hz, 2H), 3.96 (br, 1H), 3.58 − 3.47 (m, 1H), 3.14 (s, 1H), 2.66 (br, 1H), 2.45 (dd, J = 6.7, 3.7 Hz, 1H), 2.09 (qd, J = 12.2, 3.9 Hz, 1H), 1.82 (d, J = 12.1 Hz, 1H), 1.73 (d, J = 13.2 Hz, 1H), 1.43 − 1.29 (m, 1H), 0.92 − 0.85 (m, 2H), 0.72 (s, 2H). |
| | 1-cyclopropyl-1-[(3R)-1-(2-oxo-2,3- | |
| | dihydropyrimidin-4-yl)piperidin-3-yl]- | |
| | 3-[(3-phenyl-1,2-oxazol-5- | |
| | yl)methyl]urea | |
| (54) | 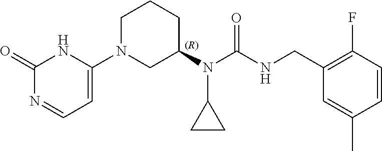 3-cyclopropyl-1-[(2-fluoro-5- | LC-MS: m/z = 400.4 (M + H) 1H NMR: ¹H NMR (400 MHz, dmso) δ 10.42 (s, 1H), 7.41 (d, J = 6.9 Hz, 1H), 7.13 − 6.96 (m, 3H), 6.81 (t, J = 5.9 Hz, 1H), 5.99 (d, J = 7.4 Hz, 1H), 4.56 (br, 1H), 4.29 (d, J = 5.8 Hz, 2H), 3.97 (br, 1H), 3.56 − 3.47 (m, 1H), 3.17 (br, 1H), 2.69 (br, 1H), 2.47 (dd, J = 6.7, 3.7 Hz, 1H), 2.27 (s, 3H), 2.10 (qd, J = 12.5, 4.1 Hz, 1H), 1.85 − 1.70 (m, 2H), 1.44 − 1.33 (m, 1H), 0.89 (dd, J = 6.4, 2.6 Hz, 2H), 0.68 (s, 2H). |
| | methylphenyl)methyl]-3-[(3R)-1-(2- | |
| | oxo-2,3-dihydropyrimidin-4- | |
| | yl)piperidin-3-yl]urea | |
| (55) | 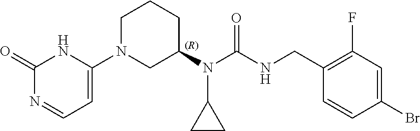 1-[(4-bromo-2-fluorophenyl)methyl]- 3-cyclopropyl-3-[(3R)-1-(2-oxo-2,3- dihydropyrimidin-4-yl)piperidin-3- yl]urea | LC-MS: m/z = 464.3 (M + H) 1H NMR: ¹H NMR (400 MHz, dmso) δ 10.42 (s, 1H), 7.47 (dd, J = 9.8, 1.9 Hz, 1H), 7.40 (dt, J = 8.3, 2.5 Hz, 2H), 7.27 (t, J = 8.2 Hz, 1H), 6.93 (t, J = 5.9 Hz, 1H), 5.98 (d, J = 7.4 Hz, 1H), 4.64 (br, 1H), 4.28 (d, J = 5.8 Hz, 2H), 3.98 (br, 1H), 3.51 (tt, J = 11.8, 3.9 Hz, 1H), 3.16 (s, 1H), 2.68 (br, 1H), 2.46 (dt, J = 6.9, 3.7 Hz, 1H), 2.08 (qd, J = 12.4, 4.0 Hz, 1H), 1.80 (d, J = 12.2 Hz, 1H), 1.74 (d, J = 13.5 Hz, 1H), 1.36 (tdd, J = 13.1, 9.1, 4.1 Hz, 1H), 0.89 (dd, J = 6.9, 2.4 Hz, 2H), 0.68 (s, 2H). |
| (56) | 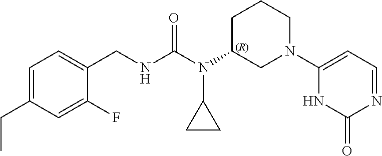 1-cyclopropyl-3-[(4-ethyl-2- fluorophenyl)methyl]-1-[(3R)-1-(2- oxo-2,3-dihydropyrimidin-4- | LC-MS: m/z = 414.5 (M + H) 1H NMR: ¹H NMR (400 MHz, dmso) δ 10.42 (s, 1H), 7.40 (d, J = 7.3 Hz, 1H), 7.21 (t, J = 8.0 Hz, 1H), 7.05 − 6.95 (m, 2H), 6.81 (t, J = 5.9 Hz, 1H), 5.99 (d, J = 7.4 Hz, 1H), 4.66 (br, 1H), 4.29 (d, J = 5.8 Hz, 2H), 4.03 (br, 1H), 3.57 − 3.46 (m, 1H), 3.16 (s, 1H), 2.71 (br, 1H), 2.59 (q, J = 7.6 Hz, 2H), 2.48 − 2.43 (m, 1H), 2.09 (qd, J = 12.4, 4.0 Hz, 1H), 1.80 (d, J = 12.0 Hz, 1H), 1.74 (d, J = 13.5 Hz, 1H), 1.44 − 1.29 (m, 1H), 1.16 (t, J = 7.6 Hz, 3H), 0.88 (d, J = 6.5 Hz, 2H), 0.68 (s, 2H). |
| | yl)piperidin-3-yl]urea | |
| (57) | 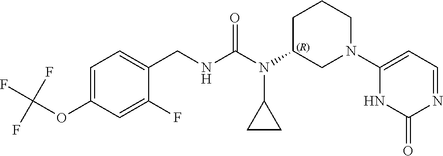 1-cyclopropyl-3-{[2-fluoro-4- (trifluoromethoxy)phenyl]methyl}-1- | LC-MS: m/z = 470.4 (M + H) 1H NMR: ¹H NMR (400 MHz, dmso) δ 10.42 (s, 1H), 7.42 (q, J = 8.2 Hz, 2H), 7.32 (d, J = 10.4 Hz, 1H), 7.28 − 7.21 (m, 1H), 6.96 (t, J = 5.9 Hz, 1H), 5.99 (d, J = 7.4 Hz, 1H), 4.67 (br, 1H), 4.33 (d, J = 5.8 Hz, 2H), 3.97 (br, 1H), 3.57 − 3.47 (m, 1H), 3.15 (s, 1H), 2.67 (br, 1H), 2.47 (dd, J = 6.8, 3.7 Hz, 1H), 2.15 − 2.02 (m, 1H), 1.81 (d, J = 12.1 Hz, 1H), 1.74 (d, J = 13.3 Hz, 1H), 1.44 − 1.33 (m, 1H), 0.89 (d, J = 6.4 Hz, 2H), 0.69 (s, 2H). |
| | [(3R)-1-(2-oxo-2,3-dihydropyrimidin- | |
| | 4-yl)piperidin-3-yl]urea | |
| (58) | 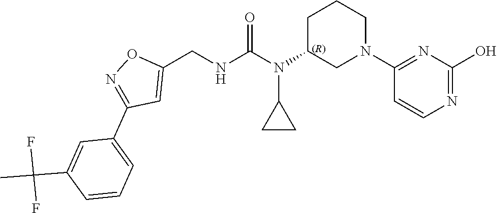 | LC-MS: m/z = 499.4 (M + H) 1H NMR: 1H NMR (400 MHz, dmso) δ 10.43 (s, 1H), 8.04 − 7.97 (m, 2H), 7.72 − 7.59 (m, 2H), 7.40 (d, J = 7.4 Hz, 1H), 7.09 (t, J = 5.9 Hz, 1H), 6.91 (d, J = 3.8 Hz, 1H), 5.99 (d, J = 7.5 Hz, 1H), 4.64 (br, 1H), 4.46 (d, J = 5.8 Hz, 2H), 3.99 (s, 1H), 3.60 − 3.50 (m, 1H), 3.16 (br, 1H), 2.67 (s, 1H), 2.47 (dd, J = 6.8, 3.7 Hz, 1H), 2.15 − 2.01 (m, 1H), 2.03 (t, J = 19.1 Hz, 3H), 1.83 (d, J = 13.1 Hz, 1H), 1.75 (d, J = 13.4 Hz, 1H), 1.39 (m, 1H), 0.90 (d, J = 6.3 Hz, 2H), 0.74 (s, 2H). |
| | 1-cyclopropyl-3-({3-[3-(1,1- | |
| | difluoroethyl)phenyl]-1,2-oxazol-5- | |
| | yl}methyl)-1-[(3R)-1-(2- | |
| | hydroxypyrimidin-4-yl)piperidin-3- | |
| | yl]urea | |
| (59) | 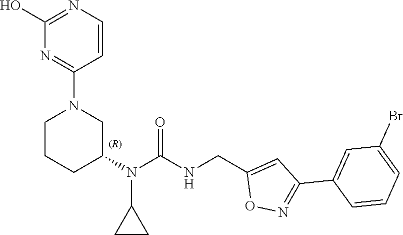 | LC-MS: m/z = 513.3 (M + H) 1H NMR: ¹H NMR (400 MHz, dmso) δ 8.05 (t, J = 1.8 Hz, 0H), 7.88 (dt, J = 7.8, 1.4 Hz, 0H), 7.68 (d, J = 7.7 Hz, 0H), 7.50 (s, 1H), 7.46 (t, J = 7.9 Hz, 0H), 7.09 (t, J = 5.9 Hz, 0H), 6.47 (s, 0H), 6.10 (d, J = 7.5 Hz, 0H), 4.43 (d, J = 5.8 Hz, 1H), 3.66 − 3.52 (m, 0H), 2.18 − 2.04 (m, 0H), 1.89 − 1.71 (m, 1H), 1.52 − 1.34 (m, 1H), 0.89 (d, J = 6.7 Hz, 1H), 0.73 (s, 1H). |
| | 3-{[3-(3-bromophenyl)-1,2-oxazol-5- | |
| | yl]methyl}-1-cyclopropyl-1-[(3R)-1- | |
| | (2-hydroxypyrimidin-4-yl)piperidin-3- | |
| | yl]urea | |
| (60) | 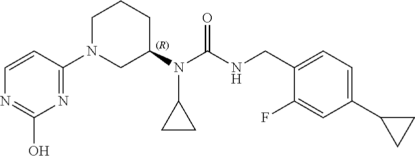 3-cyclopropyl-1-[(4-cyclopropyl-2- fluorophenyl)methyl]-3-[(3R)-1-(2- | LC-MS: m/z = 426.4 (M + H) 1H NMR: ¹H NMR (400 MHz, dmso) δ 10.42 (s, 1H), 7.40 (d, J = 7.3 Hz, 1H), 7.17 (t, J = 8.1 Hz, 1H), 6.90 (dd, J = 7.9, 1.8 Hz, 1H), 6.88 − 6.75 (m, 2H), 5.99 (d, J = 7.4 Hz, 1H), 4.70 (bs, 1H), 4.26 (d, J = 5.8 Hz, 2H), 3.96 (bs, 1H), 3.51 (t, J = 11.8 Hz, 1H), 3.17 (bs, 1H), 2.68 (bs, 1H), 2.46 (dq, J = 6.8, 3.4 Hz, 1H), 2.08 (tt, J = 12.5, 6.3 Hz, 1H), 1.90 (tt, J = 8.4, 5.1 Hz, 1H), 1.84 − 1.70 (m, 2H), 1.43 − 1.32 (m, 1H), 0.98 − 0.84 (m, 4H), 0.74 − 0.59 (m, 4H). |
| | hydroxypyrimidin-4-yl)piperidin-3- | |
| | yl]urea | |

### Examples 61-62

The compounds in the table below were prepared from intermediates such as Q1, itself prepared according to General Procedure 8, and an appropriately substituted heteroarene according to an appropriate modification of General Procedure 10 given below.

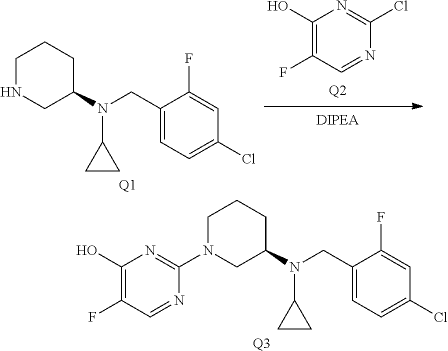

### General Procedure 10

To a solution of Q1 (35 mg, 107 umol) in DMF (1 mL) was added DIPEA (28 uL, 161 umol) and an appropriately substituted heteroarene (118 umol). The solution was heated at 60 C for one hour at which time LCMS indicated consumption of starting material. The product was then directly purified by preparative reverse phase HPLC.

| Example | Structure and name | Data |
|---|---|---|
| (61) | 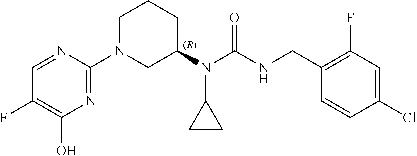 1-[(4-chloro-2-fluorophenyl)methyl]- | LC-MS: m/z = 438.3 (M + H) 1H NMR: (400 MHz, dmso) δ 11.74 (s, 1H), 7.83 (s, 1H), 7.40 − 7.23 (m, 3H), 6.92 (t, J = 5.9 Hz, 1H), 4.35 − 4.23 (m, 4H), 3.52 (ddt, J = 11.8, 7.8, 3.9 Hz, 1H), 3.22 (t, J = 12.0 Hz, 1H), 2.73 (td, J = 13.0, 2.6 Hz, 1H), 2.47 (dt, J = 6.9, 3.7 Hz, 1H), 2.05 (qd, J = 12.5, 4.1 Hz, 1H), 1.77 (d, J = 12.3 Hz, 1H), 1.70 (d, J = 13.4 Hz, 1H), 1.50 − 1.36 (m, 1H), 0.88 (ddt, J = 6.8, 4.4, 2.1 Hz, 2H), 0.68 (h, J = 3.3 Hz, 2H). |
| | 3-cyclopropyl-3-[(3R)-1-(5-fluoro-4- | |
| | hydroxypyrimidin-2-yl)piperidin-3- | |
| | yl]urea | |
| (62) | 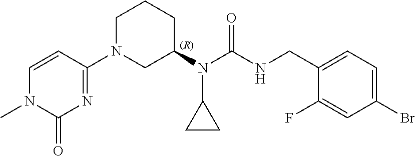 | LC-MS: m/z = 478.3 (M + H) 1H NMR: (400 MHz, dmso) δ 7.65 (d, J = 7.5 Hz, 1H), 7.45 (dd, J = 9.8, 1.9 Hz, 1H), 7.39 (dd, J = 8.2, 2.0 Hz, 1H), 7.25 (t, J = 8.2 Hz, 1H), 6.91 (t, J = 5.9 Hz, 1H), 6.63 (s, 0H), 6.05 (d, J = 7.5 Hz, 1H), 4.26 (d, J = 5.8 Hz, 2H), 3.53 − 3.42 (m, 1H), 3.20 (s, 3H), 2.69 (s, 1H), 2.49 − 2.41 (m, 3H), 2.07 (qd, J = 12.5, 4.0 Hz, 1H), 1.81 − 1.67 (m, 2H), 1.34 (tdd, J = 13.3, 9.1, 4.1 Hz, 1H), 0.90 − 0.81 (m, 2H), 0.66 (s, 2H). |
| | 1-[(4-bromo-2-fluorophenyl)methyl]- | |
| | 3-cyclopropyl-3-[(3R)-1-(1-methyl-2- | |
| | oxo-1,2-dihydropyrimidin-4- | |
| | yl)piperidin-3-yl]urea | |

Additional compounds recited in the following table were prepared by adapting the experimental procedures recited above:

| Example | Structure and name | Data |
|---|---|---|
| (63) | 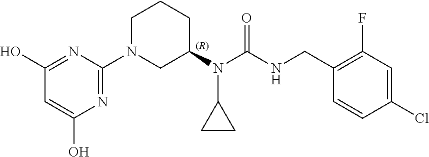 3-[(4-chloro-2-fluorophenyl)methyl]- 1-cyclopropyl-1-[(3R)-1-(4,6- | LC-MS: m/z = 436.2 (M + H) 1H NMR: 1H NMR (400 MHz, DMSO) δ 10.63 (s, 2H), 8.15 (s, 0.1H), 7.37 (dd, J = 10.1, 1.9 Hz, 1H), 7.32 (d, J = 8.1 Hz, 1H), 7.27 (dd, J = 8.3, 1.9 Hz, 1H), 6.93 (t, J = 5.8 Hz, 1H), 4.71 (s, 1H), 4.30 (t, J = 10.3 Hz, 4H), 3.52 (dd, J = 15.3, 8.1 Hz, 1H), 3.17 (t, J = 12.0 Hz, 1H), 2.67 (t, J = 12.3 Hz, 1H), 2.46 (d, J = 3.3 Hz, 1H), 2.06 − 1.94 (m, 1H), 1.73 (dd, J = 28.2, 11.9 Hz, 2H), 1.40 (dd, J = 26.1, 13.2 Hz, 1H), 0.88 (d, J = 6.2 Hz, 2H), 0.69 (dd, J = 9.7, 6.5 Hz, 2H). |
| | dihydroxypyrimidin-2-yl)piperidin-3- | |
| | yl]urea | |
| (64) | 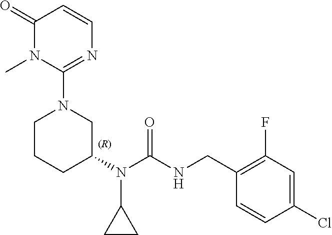 | LC-MS: m/z = 434.2 (M + H) 1H NMR: 1H NMR (400 MHz, MeOD) δ 7.74 (d, J = 6.4 Hz, 1H), 7.38 − 7.28 (m, 1H), 7.22 − 7.09 (m, 2H), 6.93 (t, J = 5.8 Hz, 1H), 6.07 (d, J = 6.4 Hz, 1H), 4.50 − 4.32 (m, 2H), 4.09 − 3.95 (m, 1H), 3.59 − 3.49 (m, 2H), 3.47 (s, 3H), 3.12 (t, J = 11.7 Hz, 1H), 2.86 − 2.73 (m, 1H), 2.56 − 2.45 (m, 1H), 2.20 − 2.07 (m, 1H), 1.97 (d, J = 11.3 Hz, 1H), 1.88 (d, J = 12.7 Hz, 1H), 1.82 − 1.67 (m, 1H), 1.01 − 0.89 (m, 2H), 0.82 − 0.70 (m, 2H). |
| | 1-[(4-chloro-2-fluorophenyl)methyl]- | |
| | 3-cyclopropyl-3-[(3R)-1-(1-methyl-6- | |
| | oxo-1,6-dihydropyrimidin-2- | |
| | yl)piperidin-3-yl]urea | |
| (65) | 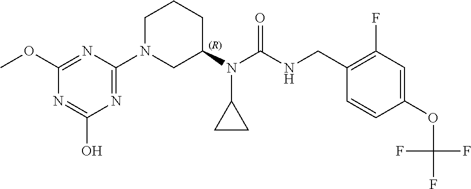 | LC-MS: m/z = 501.3 (M + H) 1H NMR: 1H NMR (400 MHz, MeOD) δ 7.44 (t, J = 8.5 Hz, 1H), 7.09 (t, J = 8.6 Hz, 2H), 6.95 (t, J = 5.7 Hz, 1H), 4.71 (s, 2H), 4.44 (s, 2H), 3.97 (s, 3H), 3.65 (s, 1H), 3.48 − 3.31 (m, 1H), 2.80 (s, 1H), 2.58 (s, 1H), 2.33 − 2.17 (m, 1H), 1.99 − 1.77 (m, 2H), 1.53 (q, J = 13.0 Hz, 1H), 0.96 (s, 2H), 0.77 (s, 2H). |
| | 1-cyclopropyl-3-{[2-fluoro-4- | |
| | (trifluoromethoxy)phenyl]methyl}-1- | |
| | [(3R)-1-(4-hydroxy-6-methoxy-1,3,5- | |
| | triazin-2-yl)piperidin-3-yl]urea | |
| (66) | 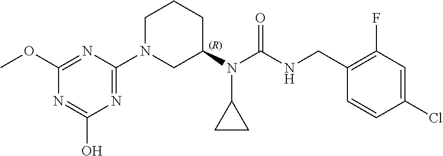 | LC-MS: m/z = 451.2 (M + H) 1H NMR: 1H NMR (400 MHz, MeOD) δ 7.32 (t, J = 8.3 Hz, 1H), 7.16 (dd, J = 8.7, 3.2 Hz, 2H), 6.91 (t, J = 5.8 Hz, 1H), 4.70 (s, 2H), 4.40 (s, 2H), 3.97 (s, 3H), 3.69 (d, J = 27.0 Hz, 1H), 3.31 (s, 1H), 2.80 (s, 1H), 2.57 (s, 1H), 2.32 − 2.15 (m, 1H), 1.95 − 1.78 (m, 2H), 1.52 (q, J = 13.1 Hz, 1H), 0.95 (s, 2H), 0.76 (s, 2H). |
| | 3-[(4-chloro-2-fluorophenyl)methyl]- | |
| | 1-cyclopropyl-1-[(3R)-1-(4-hydroxy- | |
| | 6-methoxy-1,3,5-triazin-2- | |
| | yl)piperidin-3-yl]urea | |
| (67) | 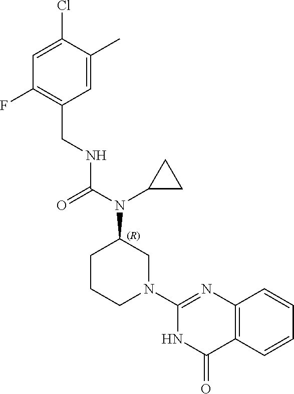 | LC-MS: m/z = 484.2 (M + H) 1H NMR: 1H NMR (400 MHz, MeOD) δ 8.00 (d, J = 7.9 Hz, 1H), 7.61 (t, J = 7.7 Hz, 1H), 7.39 (d, J = 8.2 Hz, 1H), 7.25 (dd, J = 16.0, 7.8 Hz, 2H), 7.14 (d, J = 9.8 Hz, 1H), 6.95 (s, 0.5H), 4.50 − 4.29 (m, 4H), 3.78 (t, J = 11.8 Hz, 1H), 3.37 (t, J = 12.0 Hz, 1H), 2.91 (t, J = 12.3 Hz, 1H), 2.57 (dd, J = 6.8, 3.2 Hz, 1H), 2.32 (s, 3H), 2.23 (dt, J = 12.5, 8.4 Hz, 1H), 1.97 (d, J = 11.9 Hz, 1H), 1.89 (d, J = 13.6 Hz, 1H), 1.66 (t, J = 13.0 Hz, 1H), 0.96 (d, J = 6.8 Hz, 2H), 0.80 (d, J = 3.2 Hz, 2H). |
| | 3-[(4-chloro-2-fluoro-5- | |
| | methylphenyl)methyl]-1-cyclopropyl- | |
| | 1-[(3R)-1-(4-oxo-3,4- | |
| | dihydroquinazolin-2-yl)piperidin-3- | |
| | yl]urea | |
| (68) | 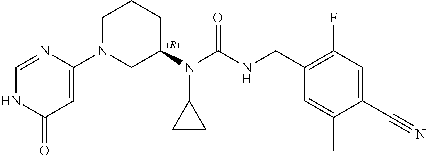 3-[(4-cyano-2-fluoro-5- | LC-MS: m/z = 425.3 (M + H) 1H NMR: 1H NMR (400 MHz, MeOD) δ 7.89 (s, 1H), 7.44 (d, J = 9.6 Hz, 1H), 7.35 (d, J = 7.1 Hz, 1H), 5.45 (s, 1H), 4.46 (d, J = 5.5 Hz, 2H), 4.33 (s, 2H), 3.69 − 3.60 (m, 1H), 3.33 (s, 1H), 2.87 − 2.75 (m, 1H), 2.63 − 2.55 (m, 1H), 2.50 (s, 3H), 2.30 − 2.17 (m, 1H), 1.96 − 1.89 (m, 1H), 1.85 − 1.78 (m, 1H), 1.60 − 1.48 (m, 1H), 0.97 (dd, J = 8.3, 6.7 Hz, 2H), 0.84 − 0.77 (m, 2H). |
| | methylphenyl)methyl]-1-cyclopropyl- | |
| | 1-[(3R)-1-(6-oxo-1,6- | |
| | dihydropyrimidin-4-yl)piperidin-3- | |
| | yl]urea | |
| (69) | 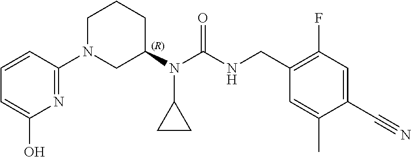 3-[(4-cyano-2-fluoro-5- | LC-MS: m/z = 424.3 (M + H) 1H NMR: 1H NMR (400 MHz, MeOD) δ 7.45 − 7.35 (m, 3H), 7.05 (t, J = 5.7 Hz, 1H), 5.87 (d, J = 8.5 Hz, 1H), 5.83 (d, J = 7.9 Hz, 1H), 4.47 (dd, J = 9.9, 5.8 Hz, 2H), 3.95 − 3.87 (m, 1H), 3.72 (d, J = 11.8 Hz, 2H), 3.08 (t, J = 11.7 Hz, 1H), 2.82 − 2.73 (m, 1H), 2.56 − 2.48 (m, 4H), 2.18 − 2.09 (m, 1H), 1.97 (d, J = 10.4 Hz, 1H), 1.86 (d, J = 13.5 Hz, 1H), 1.72 − 1.63 (m, 1H), 0.97 (dd, J = 4.2, 2.4 Hz, 2H), 0.84 − 0.72 (m, 2H). |
| | methylphenyl)methyl]-1-cyclopropyl- | |
| | 1-[(3R)-1-(6-hydroxypyridin-2- | |
| | yl)piperidin-3-yl]urea | |
| (70) | 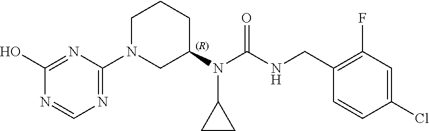 3-[(4-chloro-2-fluorophenyl)methyl]- 1-cyclopropyl-1-[(3R)-1-(4-hydroxy- | LC-MS: m/z = 421.1 (M + H) 1H NMR: 1H NMR (400 MHz, MeOD) δ 8.06 (s, 1H), 7.32 (t, J = 8.3 Hz, 1H), 7.20 − 7.11 (m, 2H), 4.76 (dd, J = 27.6, 11.2 Hz, 2H), 4.41 (s, 2H), 3.59 (d, J = 28.4 Hz, 1H), 3.38 (dd, J = 26.0, 12.3 Hz, 1H), 2.88 − 2.69 (m, 1H), 2.64 − 2.49 (m, 1H), 2.24 (dd, J = 12.6, 3.9 Hz, 1H), 1.95 − 1.78 (m, 2H), 1.53 (d, J = 13.2 Hz, 1H), 0.87 (dd, J = 52.1, 22.0 Hz, 4H). |
| | 1,3,5-triazin-2-yl)piperidin-3-yl]urea | |

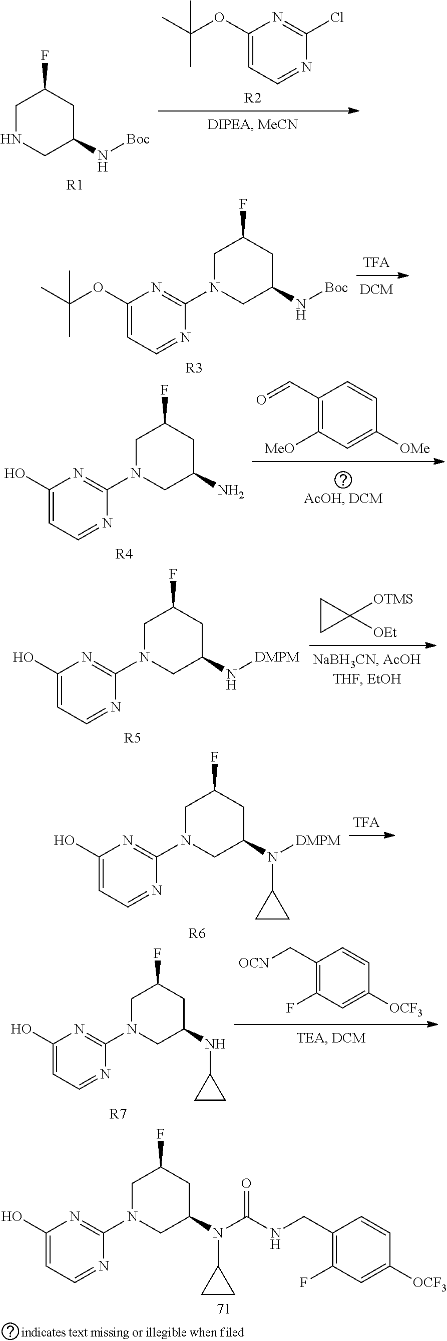

### Step 1

To a mixture of R1 (100 mg, 0.458 mmol) and DIPEA (119 mg, 0.916 mmol) in anhydrous MeCN (15 mL) R2 (103 mg, 0.550 mmol) or an appropriately substituted heteroarene was added. The resulting mixture was stirred at 90° C. for 16 hrs under N₂ atmosphere. The mixture was then diluted with EtOAc (30 mL), washed with water (30 mL) and brine (30 mL). The organic layer was separated, dried over anhydrous Na₂SO₄ and concentrated to dryness. The residue was purified by column chromatography on silica gel (eluted with PE:EtOAc=10:1 to 3:1) to afford R3 (102 mg, 60.4% yield) as a colorless oil. LC/MS (ESI) m/z: 369 (M+H)⁺.

### Step 2

To a solution of R3 (102 mg, 0.276 mmol) in DCM (4 mL) TFA (1 mL) was added at 0° C. and the reaction was stirred at room temperature for 2 hours. The mixture was then concentrated under reduced pressure to give crude R4 (TFA salt) as a colorless oil which was used for next step without any further purification. LC/MS (ESI) m/z: 213 (M+H)⁺.

### Step 3

To a solution of R4 (0.276 mmol) in anhydrous DCM (8 mL) 2,4-dimethoxybenzaldehyde (49 mg, 0.291 mmol) and AcOH (67 mg, 1.104 mmol) were added at room temperature. The mixture was stirred at room temperature for 1.5 hours, before NaBH(OAc)₃ (117.3 mg, 0.554 mmol) was added at 0° C. The resulting mixture was then stirred at room temperature overnight. After the reaction was completed, the mixture was quenched with saturated NaHCO₃ aqueous (20 mL) to adjust pH=8 and extracted with DCM (20 mL*4). The combined organic layers were washed with brine (25 mL), dried over anhydrous Na₂SO₄ and concentrated to dryness under reduced pressure. The residue was purified by chromatography on silica gel (DCM:MeOH=50:1 to 15:1) to give R5 (60 mg, 60.1% yield) as a colorless oil. LC/MS (ESI) m/z: 363 (M+H)⁺.

### Step 4

To a mixture of R5 (60 mg, 0.166 mmol) and AcOH (99.4 mg, 1.66 mmol) in THF (12 mL) and EtOH (6 mL), (1-ethoxycyclopropoxy)trimethylsilane (86.6 mg, 0.500 mmol) was added followed by the addition of NaBH₃CN (31.2 mg, 0.500 mmol). The resulting mixture was stirred at 80° C. under N₂ atmosphere for 4 hours. The mixture was then cooled to room temperature and concentrated to dryness. The residue was diluted with DCM (15 mL), poured into saturated NaHCO₃ (50 mL) to adjust pH=8. The mixture was then extracted with DCM (20 mL*2), and the combined organic layers were washed with brine (20 mL), dried over anhydrous Na₂SO₄, filtered and concentrated to dryness. The crude product was purified by column chromatography on silica gel (DCM:MeOH=100:1 to 20:1) to give R6 (62 mg, 92.9% yield) as a colorless oil. LC/MS (ESI) m/z: 403 (M+H)⁺.

### Step 5

R6 (62.0 mg, 0.154 mmol) was added into TFA (5 mL) portionwise at 0° C. under N₂ atmosphere, and the resulting mixture was stirred at 80° C. for 4 hours. The reaction mixture was cooled down to room temperature and concentrated under reduced pressure to give crude R7 (TFA salt) as a purple oil which was used for next step without any further purification. LC/MS (ESI) m/z: 253 (M+H)⁺.

### Step 6

To a mixture of R7 (0.154 mmol) and TEA (47 mg, 0.462 mmol) in anhydrous DCM (6 mL) a solution of isocyanate (0.169 mmol) in anhydrous DCM (2 mL) was added dropwise over 5 minutes at 0° C. under N₂ atmosphere, and the resulting mixture was then stirred at room temperature for 1 hour. The reaction mixture was concentrated under reduced pressure, and the residue was dissolved in EtOAc (10 mL) and washed with water (15 mL) and brine (15 mL). The organic layer was separated, dried over anhydrous Na₂SO₄ and concentrated to dryness. The residue was purified via prep-HPLC (Gemini 5 μm C18 250*21.2 mm, H₂O/MeCN (5-95%)/0.1% NH₃·H₂O) to give 73 (25 mg, 33.29% yield) as a white solid. LC/MS (ESI) m/z: 488 (M+H)⁺. ¹H NMR (400 MHz, MeOD) δ 7.65 (s, 1H), 7.45 (t, J=8.6 Hz, 1H), 7.13-6.93 (m, 3H), 5.80 (d, J=6.6 Hz, 1H), 4.67-4.53 (m, 2H), 4.45 (d, J=5.4 Hz, 2H), 4.21-4.15 (m, 1H), 3.82-3.69 (m, 1H), 2.89-2.79 (m, 1H), 2.62-2.53 (m, 1H), 2.43-2.32 (m, 2H), 1.01-0.92 (m, 2H), 0.86-0.77 (m, 2H).

### INCORPORATION BY REFERENCE

All of the U.S. patents and U.S. and PCT patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A compound of Formula (I): 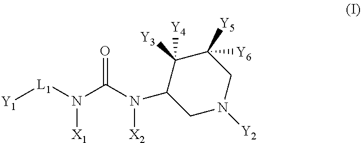
wherein:
L₁ is absent or selected from -alkyl-, -cycloalkyl-, and -heteroaryl-CH₂—;
X₁ and X₂ are independently selected from —H, alkyl, cycloalkyl, and alkyl-cycloalkyl; provided that X₁ and X₂ are not both —H;
Y₁ is optionally substituted aryl; and
Y₂ is selected from optionally substituted pyridonyl, optionally substituted pyrimidinoyl, optionally substituted pyrazinonyl, optionally substituted triazinonyl, and optionally substituted quinazolinonyl;
Y₃, Y₄, Y₅, and Y₆ are independently selected from —H and halide;
or a pharmaceutically acceptable salt thereof.

2. The compound of claim 1, wherein one of X₁ and X₂ is —H; and the other of X₁ and X₂ is selected from C₁-C₄ alkyl, cycloalkyl, and alkyl-cycloalkyl.

3. The compound of claim 2, wherein one of X₁ and X₂ is —H; and the other of X₁ and X₂ is selected from C₁-C₄ alkyl and cycloalkyl.

4. The compound of claim 3, wherein X₁ is —H; and X₂ is —CH₃.

5. The compound of claim 3, wherein X₂ is —H; and X₁ is —CH₃.

6. The compound of claim 3, wherein X₁ is —H; and X₂ is

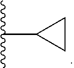 [SMILES: *C1CC1]

7. The compound of claim 3, wherein X₂ is —H; and X₁ is

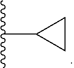 [SMILES: *C1CC1]

8. The compound of any one of claims 1-7, wherein L₁ is absent.

9. The compound of any one of claims 1-7, wherein L₁ is selected from —C₁-C₄ alkyl-, -cycloalkyl-, and -heteroaryl-CH₂—.

10. The compound of claim 9, wherein L₁ is-CH₂—.

11. The compound of claim 9, wherein L₁ is

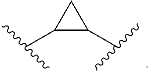 [SMILES: *C1CC1*]

12. The compound of claim 11, wherein L₁ is selected from 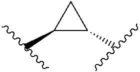 [SMILES: *[C@H]1C[C@@H]1*]
and

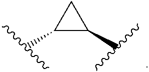 [SMILES: *[C@@H]1C[C@H]1*]

13. The compound of claim 9, wherein L₁ is selected from

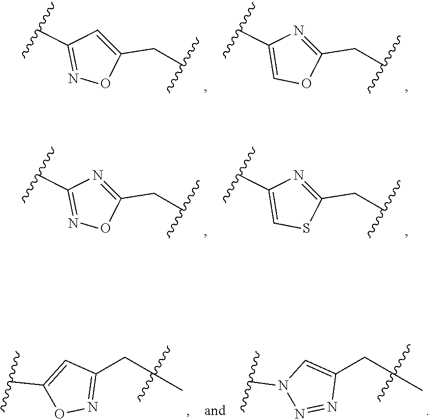 [SMILES: *Cc1cc(*)no1 ; *Cc1nc(*)co1 ; *Cc1nc(*)no1 ; *Cc1nc(*)cs1 ; *Cc1cc(*)on1 ; *Cc1cn(*)nn1]

14. The compound of any one of claims 1-13 having a structure selected from:

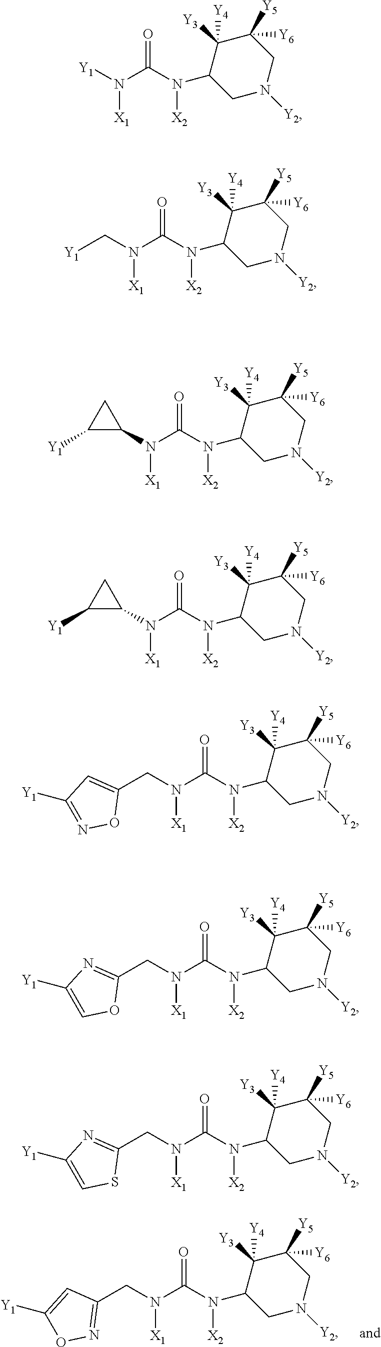

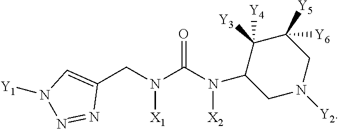

15. The compound of any one of claims 1-14, wherein Y₁ is unsubstituted aryl.

16. The compound of claim 15, wherein Y₁ is unsubstituted phenyl.

17. The compound of any one of claims 1-14, wherein Y₁ is substituted aryl.

18. The compound of claim 17, wherein Y₁ is 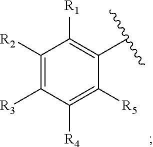
and
R₁, R₂, R₃, R₄, and R are independently selected from —H, halogen, —CN, —CF₃, —CHF₂, —CF₂CH₃, —OCF₃, —OCHF₂, alkyl, haloalkyl, hydroxyalkyl, alkenyl, alkynyl, alkoxy, alkylamino and cycloalkyl; provided that at least one of R₁, R₂, R₃, R₄, and R₅ is not —H.

19. The compound of claim 18, wherein R₁, R₂, R₃, R₄, and R₅ are independently selected from —H, —F, —Cl, —Br, —CN, —CH₃, —CH₂CH₃, —CF₃, —CHF₂, —CF₂CH₃, —C(H)(OH)(CH₃), —OCH₃, —OCF₃, —OCHF₂, and 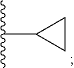 [SMILES: *C1CC1]
provided that at least one of R₁, R₂, R₃, R₄, and R is not —H.

20. The compound of claim 19, wherein R₁, R₂, R₃, R₄, and R₅ are independently selected from —H, —F, —Cl, —Br, —CN, —CH₃, —CH₂CH₃, and 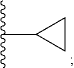 [SMILES: *C1CC1]
provided that at least one of R₁, R₂, R₃, R₄, and R₅ is not —H.

21. The compound of any one of claims 18-20, wherein two of R₁, R₂, R₃, R₄, and R₅ are not —H.

22. The compound of any one of claims 18-20, wherein three of R₁, R₂, R₃, R₄, and R₅ are not —H.

23. The compound of claim 17, wherein Y₁ is 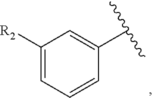
wherein R₂ is selected from —Cl, —Br, —F, —CN, —CH₃, —CH₂CH₃, —OCH₃, and —CF₂CH₃.

24. The compound of claim 17, wherein Y₁ is 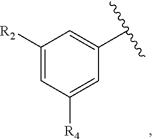
wherein R₂ and R₄ are each independently selected from —Cl, —Br, —F, —CN, —CH₃, —CH₂CH₃, —OCH₃, and —CF₂CH₃.

25. The compound of claim 17, wherein Y₁ is 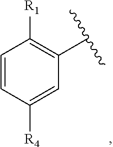
wherein R₁ and R₄ are each independently selected from —Cl, —Br, —F, —CN, —CH₃, —CH₂CH₃, —OCH₃, and —CF₂CH₃.

26. The compound of claim 17, wherein Y₁ is 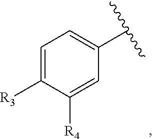
wherein R₃ and R₄ are each independently selected from —Cl, —Br, —F, —CN, —CH₃, —CH₂CH₃, —OCH₃, and —CF₂CH₃.

27. The compound of claim 17, wherein Y₁ is 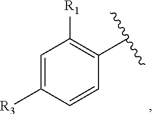
wherein R₁ and R₃ are each independently selected from —Cl, —Br, —F, —CN, —CH₃, —CH₂CH₃, —OCH₃, —OCF₃, —CF₂CH₃, and

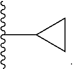 [SMILES: *C1CC1]

28. The compound of claim 27, wherein R₁ is —F; and R₃ is selected from —Cl, —Br, —F, —CN, —CH₃, —CH₂CH₃, —OCH₃, —OCF₃, —CF₂CH₃, and

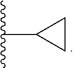 [SMILES: *C1CC1]

29. The compound of claim 28, wherein Y₁ is

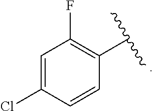 [SMILES: *c1ccc(Cl)cc1F]

30. The compound of claim 17, wherein Y₁ is 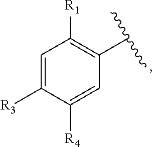
wherein R₁, R₃, and R₄ are each independently selected from —Cl, —Br, —F, —CN, —CH₃, —CH₂CH₃, —OCH₃, —OCF₃, —CF₂CH₃, —C(H)(OH)(CH₃), and

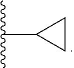 [SMILES: *C1CC1]

31. The compound of claim 30, wherein R₁ is —F; and R₃ and R₄ are each independently selected from —Cl, —Br, —F, —CN, —CH₃, —CH₂CH₃, —OCH₃, —OCF₃, —CF₂CH₃, and

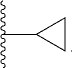 [SMILES: *C1CC1]

32. The compound of claim 31, wherein R₁ is —F; R₃ is —Cl or —F; and R₄ is selected from —Cl, —Br, —F, —CN, —CH₃, —CH₂CH₃, —OCH₃, —OCF₃, —CF₂CH₃, and

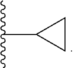 [SMILES: *C1CC1]

33. The compound of any one of claims 1-32 having the structure selected from:

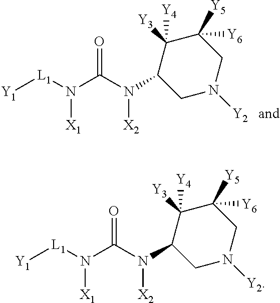

34. The compound of any one of claims 1-33, wherein Y₂ is selected from unsubstituted pyridonyl, unsubstituted pyrimidinoyl, unsubstituted pyrazinonyl, unsubstituted triazinonyl, and unsubstituted quinazolinonyl.

35. The compound of claim 34, wherein Y₂ is selected from

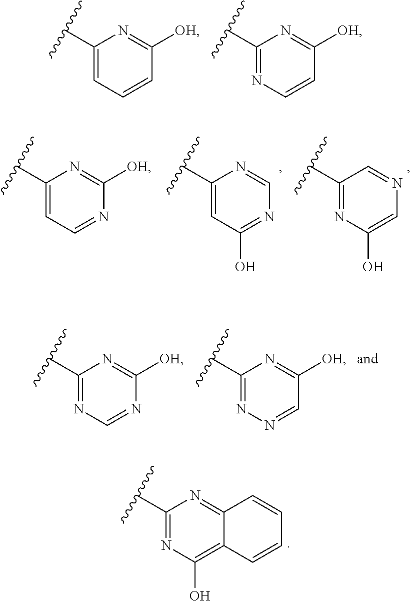

36. The compound of any one of claims 1-33, wherein Y₂ is selected from substituted pyridonyl, substituted pyrimidinoyl, substituted pyrazinonyl, substituted triazinonyl, and substituted quinazolinonyl.

37. The compound of claim 36, wherein Y₂ is 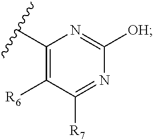
and
R₆ and R₇ are independently selected from —H, halogen, —CN, —OH, —OCF₃, —OCHF₂, —NH₂, alkyl, alkoxy, alkylamino, and cycloalkyl; provided that at least one of R₆ and R₇ is not —H; or R₆ and R₇ taken together with the carbons to which they are bonded form an unsubstituted or substituted fused C₅-C₇ cycloalkyl.

38. The compound of claim 36, wherein Y₂ is 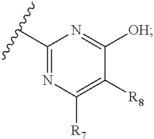
and
R₇ and R₈ are independently selected from —H, halogen, —CN, —OH, —OCF₃, —OCHF₂, —NH₂, alkyl, alkoxy, alkylamino, and cycloalkyl; provided that at least one of R₇ and R₈ is not —H; or R₇ and R₈ taken together with the carbons to which they are bonded form an unsubstituted or substituted fused C₅-C₇ cycloalkyl.

39. The compound of claim 36, wherein Y₂ is 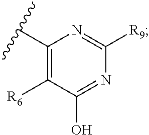
and
R₆ and R₉ are independently selected from —H, halogen, —CN, —OH, —OCF₃, —OCHF₂, —NH₂, alkyl, alkoxy, alkylamino, and cycloalkyl; provided that at least one of R₆ and R₉ is not —H.

40. The compound of claim 36, wherein Y₂ is 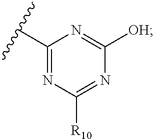
and
R₁₀ is selected from halogen, —CN, —OH, —OCF₃, —OCHF₂, —NH₂, alkyl, alkoxy, alkylamino, and cycloalkyl.

41. The compound of claim 36, wherein Y₂ is 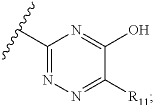
and
R₁₁ is selected from halogen, —CN, —OH, —OCF₃, —OCHF₂, —NH₂, alkyl, alkoxy, alkylamino, and cycloalkyl.

42. The compound of claim 36, wherein Y₂ is selected from

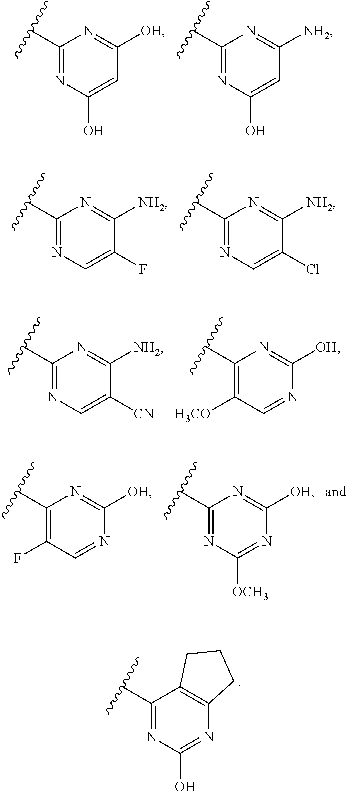

43. The compound of claim 36, wherein Y₂ is an N-substituted pyridonyl, N-substituted pyrimidinoyl, N-substituted pyrazinonyl, N-substituted triazinonyl, or N-substituted quinazolinonyl,

44. The compound of claim 43, wherein Y₂ is an N-alkyl substituted pyridonyl, N-alkyl substituted pyrimidinoyl, N-alkyl substituted pyrazinonyl, N-alkyl substituted triazinonyl, or N-alkyl substituted quinazolinonyl,

45. The compound of claim 44, wherein Y₂ is selected from

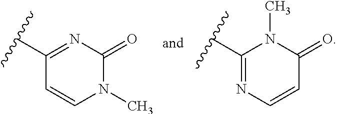

46. The compound of any one of claims 1-45, wherein Y₃, Y₄, Y₅, and Y₆ are each —H.

47. The compound of any one of claims 1-45, wherein Y₃ and Y₄ are both —H or both —F.

48. The compound of any one of claims 1-45, wherein Y₃ is —H and Y₄ is —F.

49. The compound of any one of claims 1-45, wherein Y₄ is —H and Y₃ is —F.

50. The compound of any one of claims 1-45 and 47-49, wherein Y₅ and Y₆ are both —H or both —F.

51. The compound of any one of claims 1-45 and 47-49, wherein Y₅ is —H and Y₆ is —F.

52. The compound of any one of claims 1-45 and 47-49, wherein Y₆ is —H and Y₅ is —F.

53. A compound having the structure of any one of the compounds recited in Table 1.

54. A pharmaceutical composition, comprising a compound of any one of claims 1-53; and a pharmaceutical acceptable excipient.

55. A method of treating or preventing a disease or disorder associated with a genetic defect in phenylalanine hydroxylase, comprising administering to a subject in need thereof an effective amount of a compound of any one of claims 1-53.

56. A method of treating or preventing phenylketonuria, comprising administering to a subject in need thereof an effective amount of a compound of any one of claims 1-53.

57. A method of treating or preventing hyperphenylalaninemia, comprising administering to a subject in need thereof an effective amount of a compound of any one of claims 1-53.

58. The method of any one of claims 55-57, wherein systemic phenylalanine levels in the subject are reduced.

59. A method of treating or preventing tyrosinemia (Type I, II, or III), comprising administering to a subject in need thereof an effective amount of a compound of any one of claims 1-53.

60. The method of claim 59, wherein systemic tyrosine levels in the subject are reduced.

61. A method of treating or preventing nonketotic hyperglycinemia, comprising administering to a subject in need thereof an effective amount of a compound of any one of claims 1-53.

62. The method of claim 61, wherein systemic gly cine levels in the subject are reduced.

63. A method of treating or preventing isovaleric acidemia, methylmalonic acidemia, propionic acidemia, maple syrup urine disease, DNAJC12 deficiency, urea cycle disorders, or hyperammonemia, comprising administering to a subject in need thereof an effective amount of a compound of any one of claims 1-53.

64. A method of treating or preventing diabetes, chronic kidney disease, nonalcoholic fatty liver disease, nonalcoholic steatohepatitis, metabolic syndrome, obesity related disorders, or neurodevelopmental and autism-spectrum disorders, comprising administering to a subject in need thereof an effective amount of a compound of any one of claims 1-53.

65. The method of any one of claims 55-64, wherein SLC6A19 function in the subject is inhibited.
